(19) United States
(12) Patent Application Publication
(10) Pub. No.: US 2026/0004884 A1
(43) Pub. Date: Jan. 1, 2026

(54) COMPREHENSIVE PROCESS FOR HEALTH ASSESSMENT OF AN AGRICULTURAL UNIT

(71) Applicant: Evolve Genomix, Inc., Pleasanton, CA (US)

(72) Inventors: Prasanna Thwar Krishman, Pleasanton, CA (US); Shadi Shokralla, Pleasanton, CA (US); Skaokang Zhang, Pleasanton, CA (US)

(21) Appl. No.: 19/243,452

(22) Filed: Jun. 19, 2025

Related U.S. Application Data
(60) Provisional application No. 63/662,325, filed on Jun. 20, 2024.

(51) Int. Cl. G16B 40/10; G16B 30/10

(52) U.S. Cl. CPC G16B 40/10; G16B 30/10

(57) ABSTRACT

Provided are processes and systems for determining health risks to an agricultural unit based on sets of quantitative data. Systems of the disclosure can identity creatures present in the agricultural unit (e.g., crop genomic identity, pest genomic identity) based on their nucleic acid sequences. The disclosure provides comprehensive systems trained with large amounts of quantitative data for providing an assessment of the agricultural unit.

### TECHNICAL FIELD

Monitoring of insects, nematodes, bacterial, fungal and viral pathogens and other pests in soil, in a farm and its crops. Providing early alerts based on risk assessments derived from genomic and other farm-related data that allow farmers to proactively address the risk to the crops and to the farm. Advanced genomic analysis systems for agricultural health assessment that solve specific technical problems in environmental DNA processing, including degraded sample analysis, mixed-species taxonomic classification, and real-time pathogen detection through specialized bioinformatics algorithms designed for field-collected biological samples.

### SUMMARY

Unless otherwise defined, all technical and scientific terms used herein have the same meaning as commonly understood by one of ordinary skills in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Conventional agricultural monitoring approaches suffer from several critical technical limitations that the present invention addresses. Traditional microscopic examination methods are limited by their inability to detect non-culturable microorganisms and cannot identify different life stages of pests such as eggs, larvae, and nymph stages. Existing bioinformatics pipelines fail when processing degraded environmental DNA samples because they rely on standard sequence alignment algorithms that cannot handle the unique error patterns and mixed-template amplification products common in field samples. Current machine learning approaches for biological data analysis generate frequent taxonomically impossible classifications because they lack integration of biological domain knowledge and phylogenetic constraints.

The overall health of an agricultural farm is dependent on multiple factors-innate immunity & health of the crops/plants in the farm, soil quality and the microbial diversity of the soil in the farm, weather and climatic conditions, water quality and irrigation methods, pests & beneficial insects present in the farm, the nature of pathogens and non-pathogenic microbes present in the circulating insects and inside the plants, etc. This invention is related to combining select measurable parameters to generate a screening system and a comprehensive risk score for the overall health of the farm. While some manual and automated methods can be used to gather this information such as microscopic examination of insect traps, drone-based remote monitoring of insects and plant physiology and satellite or infra-red imaging of soil, this invention is particularly about using DNA-sequencing based methods to gather information in an unbiased manner. In some aspects, the invention relates to a curated database comprising thousands of taxa that supports a screening system (for identifying taxa from a sample) and a risk assessment system (for ascribing a risk score to a farm based on the presence and/or absence or certain microorganisms in a sample). In some aspects, this invention is also about using machine learning algorithms to process the diverse datasets and generate risk scores for predicting the magnitude of risks and predicting the best interventional strategy for the specific farm.

The swift and precise identification of pests in crops and fields is crucial to prevent its damage. Nucleic acids associated with such pests can be amplified from minuscule quantities, facilitating highly specific detection through sequencing from quantities that may not yet be noticeable. Indeed, nucleic acid sequencing approaches can be used to accurately identify vector-borne pathogens afflicting a crop. The present disclosure describes a comprehensive plant, farm and soil health assessment process which includes a genomic pest and vector-borne pathogen monitoring process comprising robust datasets collected from the sequencing of insect traps, plant parts (leaves, stem, bark, flowers, fruits, roots, and others) and farm environmental samples (including but not limited to soil, air, water run-off) and soil microbial diversity data collected from microbiome sequencing of the rhizosphere of the plants. The disclosure describes processes and systems for identifying pests, e.g., insects and the pathogens present in samples collected from, e.g., a farm (or orchard, vineyard, ranch, granary, etc.) and microbes present in the soil and produces a risk score(s) for the concerned site being tested, e.g., a farm, a crop, a field through a machine learning algorithm. These risk scores can be weighed on several factors, including but not limited to the nature of the pest detected (taxa, native or foreign, resistance developed); an abundance of the pathogens detected, composition of pathogenic, and beneficial microbes in the plants. Such risk scores may roughly correlate with the economic burden that the farm may incur due to the presence of detected pests and pathogens, if not addressed timely.

In some aspects, the disclosure provides a process for screening an agricultural unit for a health risk to the agricultural unit, comprising: generating a plurality of nucleic acid sequencing reads from one or more sample(s) from the agricultural unit; screening the plurality of nucleic acid sequencing reads derived from the sample(s) gathered from the agricultural unit for a pattern that identifies the health risk to the agricultural unit, whereby the screening is performed by a computer program model trained to identify the pattern from a plurality of features, whereby one or more features comprises the abundance of the plurality of taxa found in the agricultural unit. outputting the risk score that is predictive of the health risk to the agricultural unit based on the screening.

The screening of the agricultural unit is performed by a specialized genomic pattern recognition system that includes three key components. First, the system uses a multi-layered machine learning architecture that incorporates biological knowledge about how species are related evolutionarily and how they interact ecologically. This biological knowledge prevents the system from making impossible classifications that standard machine learning models often produce when analyzing environmental samples containing DNA from multiple species. Second, the system employs a specialized algorithm for extracting features from DNA barcodes that processes genetic sequences through hierarchical clustering methods. These methods apply different weights to different species based on how well-conserved their genetic markers are across key DNA regions like Cytochrome Oxidase I, Cytochrome b, 16S rRNA, and ITS regions.

The weighting system automatically adjusts based on how degraded the environmental DNA samples are. Third, the system includes a correlation engine that analyzes biological pathways to identify relationships between pathogens, their hosts, and disease vectors across different types of samples. This correlation engine uses ecological modeling algorithms that consider how long pathogens can survive in different environments and how they spread throughout agricultural systems.

In some aspects, the disclosure provides a process for screening an agricultural unit for a health risk to the agricultural unit, comprising: screening a plurality of features processed from a sample (e.g., a physical sample) of the agricultural unit for a pattern that identifies the health risk to the agricultural unit, whereby the screening is performed by a computer program model trained to identify the pattern from a plurality of features, whereby one or more features are selected from a set of quantitative data, whereby the set of quantitative data comprises a plurality of nucleic acid sequences extracted from the agricultural unit. The health risk to the agricultural unit can be a risk of crop failure or damage, a risk of a pest infestation, a risk of a depletion of organic matter, a risk of a soil mineralization, and/or a risk of depletion of beneficial microbiota from the soil or from within the crop. In some instances, the plurality of nucleic acid sequences extracted from the agricultural unit comprises one or more, two or more, three or more, four or more, five or more, six or more, or all of a plurality of nucleic acid sequences extracted from at least one arthropod; a plurality of nucleic acid sequences extracted from at least one nematode; a plurality of nucleic acid sequences extracted from at least one bacterium; a plurality of nucleic acid sequences extracted from at least one fungus/mold/yeast; a plurality of nucleic acid sequences extracted from at least one protozoa; a plurality of nucleic acid sequences extracted from at least one archaea; and/or a plurality of nucleic acid sequences extracted from at least one virus. In some instances, the computer program model identifies a taxa in the agricultural unit based on a plurality of chromosomal or mitochondrial metabarcode nucleic acid sequences selected from the group comprising Cytochrome Oxidase I, Cytochrome b, 16S rRNA, ITS in the plurality of nucleic acid sequences extracted from the at least one arthropod. In some instances, the computer program model identifies a taxa in the agricultural unit based on a plurality of chromosomal or mitochondrial metabarcode nucleic acid sequences selected from the group comprising Cytochrome Oxidase I, Cytochrome b, 18S small subunit rRNA, and 28S large subunit rRNA in the plurality of nucleic acid sequences extracted from the at least one nematode. In some instances, the computer program model identifies a taxa in the agricultural unit based on a plurality of chromosomal or mitochondrial metabarcode nucleic acid sequences selected from the group comprising 16S rRNA, ITS in the plurality of nucleic acid sequences extracted from the at least one bacterium or archaea. In some instances, the computer program model identifies a taxa in the agricultural unit based on a plurality of chromosomal or mitochondrial metabarcode nucleic acid sequences selected from the group comprising 16S rRNA and ITS in the plurality of nucleic acid sequences extracted from the at least one fungus. In some instances, the computer program model identifies a taxa in the agricultural unit based on a plurality of chromosomal or mitochondrial metabarcode nucleic acid sequences selected from the group comprising 16S rRNA, 18S rRNA in the plurality of nucleic acid sequences extracted from the at least one protozoa. In some instances, the computer program model identifies a virus in the agricultural unit based on targeted amplicon sequencing assay. In some instances, the process identifies larvae, nymph, eggs, and mature taxa. In some instances, the processing of the sample of the agricultural unit comprises isolating a plurality of nucleic acids from the sample and processing the plurality of nucleic acids for sequencing. In some instances, the extracted nucleic acids are enriched via primer-based target amplification—e.g., in instances where the set of primers for primer-based target amplification comprises markers for insecticide resistance, antibiotic, and/or anti-fungal resistance marker. In some instances, the extracted nucleic acids are enriched via an amplification method selected from the group comprising of: PCR, RPA, NASBA, LAMP, TMA, MDA, RCA, and HAD amplification methods. In some instances, the extracted nucleic acids are enriched via hybridization-based target amplification using a panel of capture probes selected from the group comprising of: DNA capture probes, ssDNA capture probes, and RNA capture probes. In some instances, the hybridization-based target amplification is a solid-phase target capture assay, a bead-capture assay, or a liquid-phase capture assay. In some instances, the extracted nucleic acids are enriched via aptamer-based capture assay or molecular inversion probes based capture assay. In some instances, the sequencing comprises Sanger sequencing. In some instances, the sequencing comprises next-generation selected from the group consisting of: sequencing-by-synthesis, semiconductor sequencing, avidity sequencing, single-read flow-based sequencing, and sequencing-by-binding. In some instances, the sequencing comprises third-generation sequencing such as single molecule real-time sequencing (e.g., SMRT by Pacific Biosciences) or nanopore sequencing (e.g., Oxford Nanopore Technology). In some instances, the screening of the plurality of features comprises screening the plurality of nucleic acid sequences extracted from the agricultural unit and forming a first list of raw sequence reads. In some instances, the screening further comprises forming a second list of conserved metasequences present in the first list of raw sequence reads. In some instances, the computer program model produces a score from the second list of conserved metasequences. In some instances, the computer program product is trained to identify the pattern from at least one million features. In some implementations, the computer program product is trained to identify the predictive pattern with a sensitivity of 80%-90% and/or a specificity of 80%-90%.

The genomic pattern recognition system achieves 80-90% sensitivity and specificity through three key technical improvements. First, the system uses a proprietary algorithm that optimizes read thresholds by automatically adjusting based on the specific characteristics of the sequencing platform being used and how degraded the samples are. This approach eliminates the false positive results that are caused by sequencing artifacts, which are a persistent problem in conventional bioinformatics analysis pipelines. Second, the system employs phylogenetic clustering algorithms that are specifically designed to handle environmental samples containing DNA from multiple species. These algorithms overcome the technical limitations that standard BLAST-based approaches encounter when trying to process degraded DNA collected from field samples. Third, the system uses decision tree structures that incorporate biological pathway knowledge to prevent impossible species classifications that generic machine learning models commonly make when they don't understand biological relationships.

The genomic screening process uses a novel multi-stage bioinformatics pipeline that includes four key components. First, the system employs a proprietary algorithm for clustering sequences that groups raw DNA reads based on how similar they are to each other, while also accounting for the specific types of errors that occur when sequencing environmental DNA samples. Second, the system includes a consensus sequence generation module that uses weighted voting methods to handle the technical challenges that arise from mixed-template PCR products, which are commonly found in environmental samples where multiple organisms are present. Third, the system incorporates a taxonomic assignment engine that uses phylogenetic placement algorithms specifically optimized for short DNA barcode sequences. This engine solves the problem of ambiguous species assignments that conventional sequence alignment methods struggle with. Fourth, the system applies abundance estimation algorithms that correct for the biases introduced during PCR amplification and the variations in DNA extraction efficiency that occur across different types of organisms. These corrections provide quantitative accuracy improvements compared to standard DNA barcoding analysis pipelines.

Furthermore, the system uses specialized optimization algorithms tailored to different sequencing platforms and their unique technical challenges. For Ion Torrent sequencing, the system employs error correction algorithms specifically designed to handle homopolymer errors that commonly occur when sequencing environmental samples. When processing Oxford Nanopore long-read data from degraded DNA samples, the system applies quality score recalibration protocols that account for the specific error patterns of this technology. The system also includes contamination detection and removal algorithms that identify and eliminate adapter sequences, with special optimization for multiplexed environmental samples where contamination is more prevalent. Additionally, the system incorporates chimeric sequence detection protocols that address the particular challenges that arise during mixed-species PCR amplification, where multiple organism templates can create artificial hybrid sequences.

In some instances, the disclosure describes a system for screening an agricultural unit for a taxa, the system comprising: a receiving module for receiving a plurality of primary nucleic acid sequences extracted from at least one sample (e.g., a physical sample or digital sample) collected from the agricultural unit; a database comprising a plurality of nucleic acid sequences uniquely associated with a taxa, the database comprising: a plurality of nucleic acid sequences extracted from at least one arthropod; a plurality of nucleic acid sequences extracted from at least one nematode; a plurality of nucleic acid sequences extracted from at least one bacterium; a plurality of nucleic acid sequences extracted from at least one fungus/mold/yeast; a plurality of nucleic acid sequences extracted from at least one protozoa; and/or a plurality of nucleic acid sequences extracted from at least one archaea; a screening module for comparing the plurality of primary nucleic acid sequences received by the receiving module against the plurality of nucleic acid sequences uniquely associated with the taxa in the database; and an output module for outputting a list of taxa present in the at least one sample from the agricultural unit.

In some aspects, the disclosure provides a system and a process for screening an agricultural unit for one or more taxa, comprising: generating a plurality of nucleic acid sequencing reads from one or more sample(s) gathered from the agricultural unit; screening the plurality of nucleic acid sequencing reads from the one or more sample(s) gathered from the agricultural unit to identify at least one taxa from the one or more sample(s), whereby the screening is performed by a computer program model trained to identify one or more taxa from a plurality of curated against a curated database.

A specialized agricultural genomics analysis system includes at least four main components. First, the system contains a sample preprocessing module that uses advanced quality assessment algorithms specifically developed for degraded environmental samples. This module includes proprietary algorithms that can detect and correct the amplification bias that occurs during PCR when working with mixed-species communities where multiple organisms are present. Second, the system maintains a curated genomic database containing over 41,000 sequences with custom validation protocols that solve the technical problem of incorrectly classified sequences that exist in public databases. Third, the system incorporates a multi-threaded screening engine that uses parallel processing optimization designed specifically to handle the heavy computational demands of DNA barcode analysis. This engine includes memory management algorithms that prevent system slowdowns when processing large environmental datasets. Fourth, the system features a risk assessment output system that applies specialized weighted scoring algorithms based on the ecological relationships between detected species, providing technical improvements over conventional systems that only report whether organisms are present or absent.

The genomic database incorporates several innovative validation protocols to ensure data accuracy and reliability. First, the system uses phylogenetic consistency checking algorithms that automatically identify and flag sequence annotations that don't align with established taxonomic relationships. Second, the database employs automated quality assessment tools that systematically detect and remove sequences containing technical artifacts from the sequencing process. Third, the system maintains dynamic updating protocols that allow for the incorporation of newly discovered taxonomic information while preserving compatibility with existing data structures. Finally, cross-reference validation systems verify that each sequence-taxonomy pairing is supported by multiple independent scientific sources.

Any of the aforementioned systems can comprise screening modules that can further create a secondary list(s) of sequences from the primary nucleic acid sequences based on conserved metasequences listed on the database to be uniquely associated with a taxa, or based on select target amplicon sequences (e.g., when target amplicons are used to amplify a virus). Screening modules of systems of the disclosure can further create a secondary list(s) of all conserved metasequences identified by the screening module to be present in the plurality of primary nucleic acid sequences. In some instances, the computer program model produces a score from the secondary list of conserved metasequences. In some instances, the plurality of primary nucleic acid sequences extracted from the agricultural unit comprises at least two of, at least three of, at least four of, at least five of, all six of, or all of a plurality of nucleic acid sequences extracted from at least one arthropod; a plurality of nucleic acid sequences extracted from at least one nematode; a plurality of nucleic acid sequences extracted from at least one bacterium; a plurality of nucleic acid sequences extracted from at least one fungus/mold/yeast; a plurality of nucleic acid sequences extracted from at least one protozoa; a plurality of nucleic acid sequences extracted from at least one archaea; and/or a plurality of nucleic acid sequences extracted from at least one virus. In some instances, the screening module identifies a taxa in the agricultural unit based on a plurality of chromosomal or mitochondrial metabarcode nucleic acid sequences selected from the group comprising Cytochrome Oxidase I, Cytochrome b, 16S rRNA, ITS, in the plurality of nucleic acid sequences extracted from the at least one arthropod, nematode, bacterium, and/or archaea. In some instances, the screening module identifies a taxa in the agricultural unit based on a 16S rRNA nucleic acid sequence and/or an internal transcribed spacer (ITS) sequence in the plurality of nucleic acid sequences extracted from the at least one fungus. In some instances, the screening module identifies a taxa in the agricultural unit based on a 18S rRNA nucleic acid sequence in the plurality of nucleic acid sequences extracted from the at least one protozoa. In some instances, the screening module identifies larvae, nymph, eggs, and mature taxa based on the plurality of nucleic acid sequences uniquely associated with a taxa. In some instances, the output module further provides a list of all taxa identified by the screening module. In some instances, the database comprises at least 40,000 nucleic acid sequences uniquely associated with greater than 7,100 genera of bacteria and fungi. In some instances, the database comprises at least 3.4 million nucleic acid sequences uniquely associated with greater than of 510,000 genera of arthropods. In some instances, the database comprises at least 100,000 nucleic acid sequences uniquely associated with greater than of 1,000 genera of nematodes. In some instances, the database comprises at least 100,000 nucleic acid sequences uniquely associated with greater than of 1,000 genera of protozoa. In some instances, the database comprises at least 100,000 nucleic acid sequences uniquely associated with greater than of 1,000 genera of archaea. In some instances, the health risk score provides a measure of a risk of crop failure or damage, a measure of a risk of a pest infestation, a measure of a risk of a depletion of organic matter, a measure of a risk of a soil mineralization, a measure of a risk of depletion of beneficial microbiota from the soil or from within the crop.

These aspects and other features and advantages of the invention are described below in more detail.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The foregoing and other features and advantages of the present invention will be more fully understood from the following detailed description of illustrative embodiments taken in conjunction with the accompanying drawings in which:

FIG. 1 (FIG. 1) is an schematic illustrating various types of sources of nucleic acids in crop, fields, soil and agricultural farms, including arthropods, nematodes and vector-borne plant pathogens. The diagram also depicts microbes that may be detected due to their presence in irrigation and agricultural run-off water (water-borne) pathogen data.

FIG. 2 (FIG. 2) is an schematic illustrating a process of the disclosure comprising the steps of obtaining information from the source, including nucleic acids from a sample at the source (wet-lab data), calculating a risk based on the nucleic acid data, determining risk predictions (e.g., crop losses, economic burden, soil quality erosion) and corrective action recommendations (e.g., pesticides to use, fertilization strategy, plant probiotics, and other).

FIG. 3 (FIG. 3) is an schematic illustrating commonly monitored insect-guilds and the efficiency associated with different traditional benchmarking methods.

FIG. 4 (FIG. 4) is a schematic underscoring a process that targets universal meta barcode regions for insects, bacterial and fungi in genomic DNA extracted from, e.g. the sticky insect traps.

FIG. 5 (FIG. 5) is a visual schematic of a metabarcoding workflow optimized in the instantly described processes.

FIG. 6 (FIG. 6) is a pie chart describing insect distribution as detected in a study of different orchards.

FIG. 8 (FIG. 8) is a schematic of databases used by the systems of the disclosure, including insect/arthropods databases, pathogens on the insects, microbes on plant parts (both pathogens and beneficials), soil microbiome, water-borne pathogens. The schemetic also depicts the methods used by the disclosure to assess the presence of such taxa, including sequencing (DNA barcodes) based, microscopy based, drone-based imaging, sequencing (16S+ITS) based, PCR, ELISA and certain DNA-based and antigen-based assays.

FIG. 8 (FIG. 8) illustrate modules of systems of the disclosure for screening an agricultural unit (e.g., receiving module, database module, screening module, output module) and steps by which the systems screen the agricultural unit and provide a list of taxa and abundance.

FIG. 8 also illustrate modules of the system for risk assessment for the agricultural unit, including the receiving module, screening module, and output module and steps by which the systems screen the agricultural unit and provide a compounded risk score for the agricultural unit.

FIG. 9 (FIG. 9) illustrate specific steps performed by the receiving module and the screening module of a system for risk assessment for the agricultural unit of the disclosure to determine plant health risk.

FIG. 10 (FIG. 10) illustrate specific steps performed by the receiving module and the screening module of a system for risk assessment for the agricultural unit of the disclosure to determine soil health risk.

FIG. 11 (FIG. 11) illustrate specific steps performed by the receiving module and the screening module of a system for risk assessment for the agricultural unit of the disclosure to determine water (run-off) health risk.

FIG. 12 (FIG. 12) illustrate specific steps performed by the receiving module and the screening module of a system for risk assessment for the agricultural unit of the disclosure to determine pest infestation risk.

FIG. 13 (FIG. 13) is a schematic illustrating integration in the screening module of the plant health risk rating and score, the soil health risk rating and score, the water health risk rating and score, and the pest infestation risk rating and score for providing an overall farm health risk.

FIG. 14 (FIG. 14) is a heat map depicting the relative abundance of bacterial genera between soil samples.

FIG. 15 (FIG. 15) is a heat map depicting the relative abundance of fungal and protozoan genera between the soil samples.

FIG. 16 (FIG. 16) is a heat map depicting the relative abundance of microbes between the soil types.

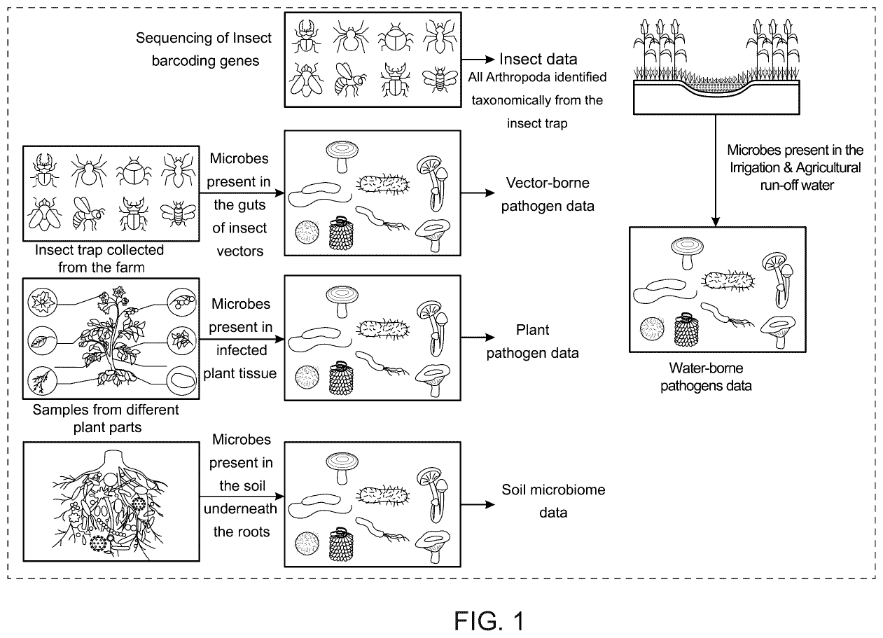

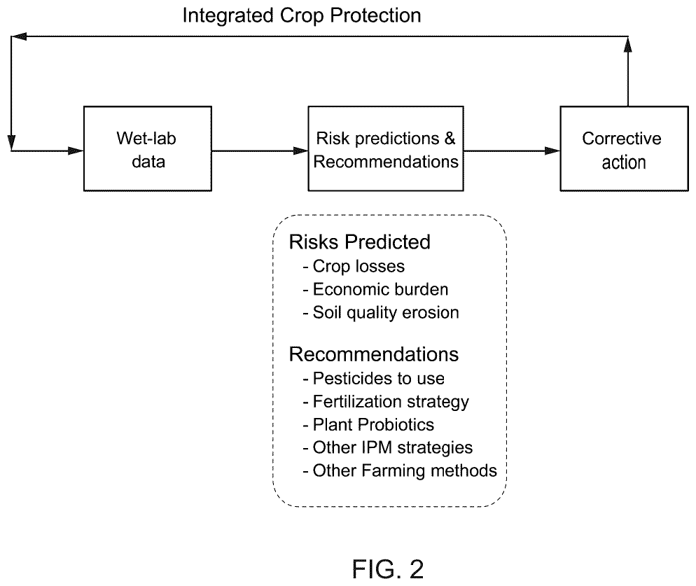

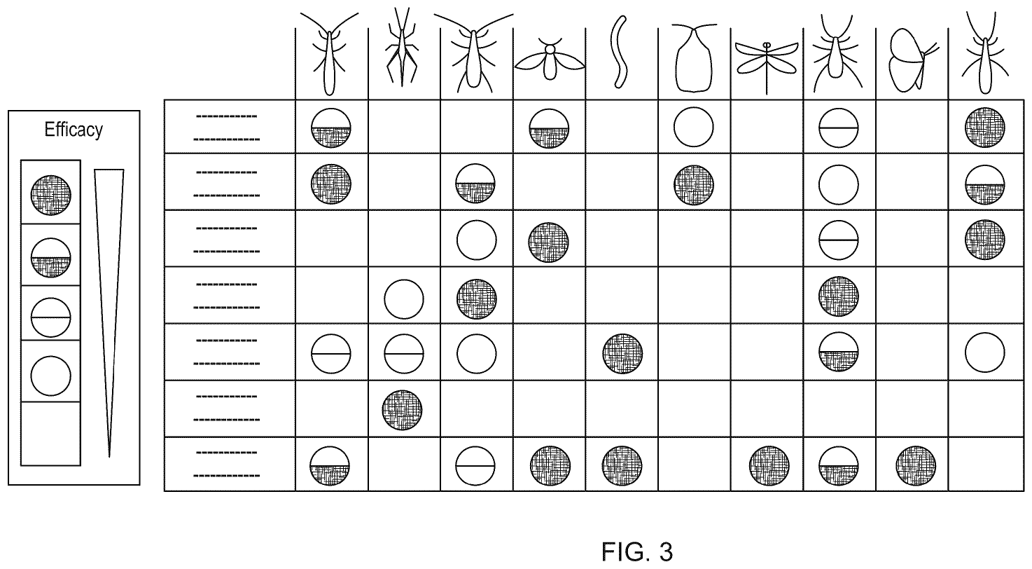

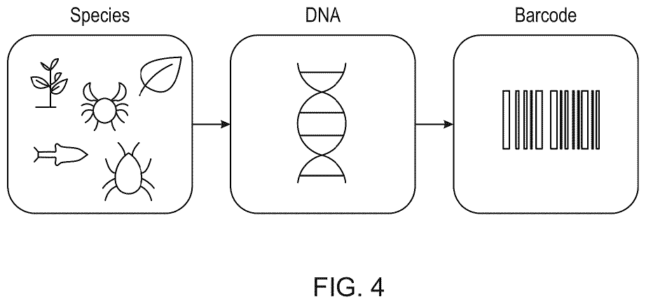

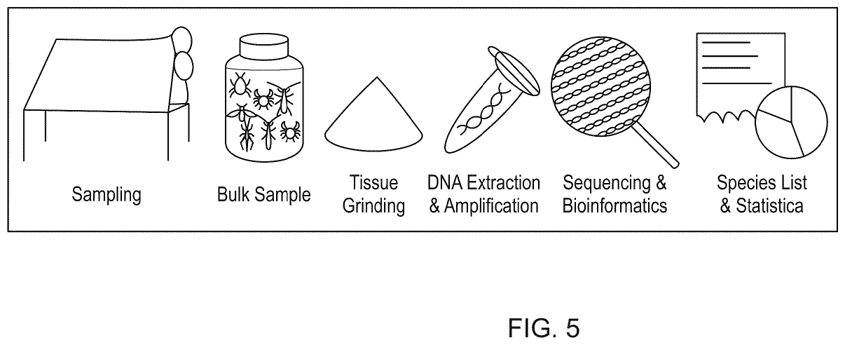

It should be understood that the drawings and pictures are not necessarily to scale.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Terminology

As used herein, the term “an agricultural unit” generally refers to land, dwellings, or other buildings occupied by living systems, including, a rhizome, a person, animal, and/or other living systems, as a unit for agricultural, agronomic, and/or animal husbandry purposes.

As used herein, the term “field” generally refers to a large, open area of land that is used for agricultural purposes. A field may be utilized for various farming activities, such as growing crops, grazing livestock, or other agricultural practices. Fields can be enclosed or open, and they serve as distinct units within a farm. Some fields are left to lie fallow or used as arable land.

As used herein, the term “crop” generally refers to a plant or plant product that is grown for a specific purpose. Crops can be cultivated extensively for profit or subsistence. They include plants grown for food, fiber, or fuel. When plants of the same species are systematically arranged in rows or other patterns, it is referred to as crop cultivation or crop field.

As used herein, the term “farm” is an area of land, typically with fields and buildings, used for raising animals and growing crops.

In summary, a field is the physical space where agricultural activities take place, while a crop specifically refers to the plants grown within that field for various purposes. A farm typically encloses one or both of crops and fields in addition to buildings and other supporting structures.

As used herein, the term “metabarcoding” is the barcoding of DNA/RNA (or eDNA/eRNA) in a manner that allows for the simultaneous identification of many taxa within the same sample.

As used herein, the term “cytochrome c oxidase I”, abbreviated COI, CO1, or COX1, COX1), refers to nucleic acid sequences encoding mitochondrial MT-CO1 genes in eukaryotes and arthropods (e.g., insects and spiders).

As used herein, the term “Internal Transcribed Spacer” or “ITS” refers to the spacer DNA between the small-subunit and large-subunit rRNA genes in bacteria, fungi and archaea.

As used herein, the term 16S and 18S refer to hypervariable ribosomal RNA (rRNA) regions in bacteria and fungus.

Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation. Specifically, a process where a first step is to determine a risk prediction (e.g., crop losses, economic burden, soil quality erosion) and second step us to make a recommendation (e.g., pesticides to use, fertilization strategy, plant probiotics, and other) doesn't necessarily mean that the risk prediction must be outputted before a specific recommendation is outputted.

“Sequence identity” or “identity” in the context of two polynucleotides sequences makes reference to the residues in the two sequences that are the same when aligned for maximum correspondence over a specified comparison window.

“Percentage of sequence identity” includes the value determined by comparing two optimally aligned sequences (greatest number of perfectly matched residues) over a comparison window, wherein the portion of the polynucleotide sequence in the comparison window May comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, the term “homology” pertains to comparative studies. Homology indicates an ancient common origin and temporal evolution and refers to structural characteristics. In comparative anatomy, it is used to compare structures in different animal species. In comparative protein biochemistry, “homology” retains the original meaning of “having a common evolutionary origin” and is used to evolutionarily define two or more proteins by locating common structural characteristics and common spatial distribution of, for instance, beta strands, helices, and folds. Accordingly, homologous protein structures are defined by spatial analyses. Measuring structural homology involves computing the geometric-topological features of a space. One approach used to generate and analyze three-dimensional (3D) protein structures is homology modeling (also called comparative modeling or knowledge-based modeling). Homology modeling works by finding similar sequences on the basis of the obvious fact that 3D similarity reflects 2D similarity. Nonetheless, it is important to note that homologous structures do not imply sequence similarity as a necessary condition. Sequence identity is the amount of characters which match exactly between two different sequences. Hereby, gaps are not counted and the measurement is relational to the shorter of the two sequences. This has the effect that sequence identity is not transitive, i.e. if sequence A=B and B=C then A is not necessarily equal C (in terms of the identity distance measure): A: AAGGCT, B: AAGGC, 11 C: AAGGCAT/Here identity (A,B)=100% (5 identical nucleotides/min (length (A),length (B))). Identity (B,C)=100%, but identity (A,C)=85% ((6 identical nucleotides/7)). So 100% identity does not mean two sequences are the same. Sequence similarity is first of all a general description of a relationship but nevertheless its more or less common practice to define similarity as an optimal matching problem (for sequence alignments or unless defined otherwise). Hereby, the optimal matching algorithm finds the minimal number of edit operations (inserts, deletes, and substitutions) in order to transform the one sequence into an exact copy of the other sequence being aligned (edit distance). Using this, the percentage sequence similarity of the examples above are sim (A,B)=60%, sim (B,C)=60%, sim (A,C)=86% (semi-global, sim=1-(edit distance/unaligned length of the shorter sequence)). But there are other ways to define similarity between two objects (e.g. using tertiary strucure of proteins).

As used herein, “about” and the term “approximately,” means the recited quantity exactly and small variations within a limited range encompassing plus or minus 10% of the recited quantity. In other words, the limited range encompassed can include ±10%, ±9%, ±8%, ±7%, ±6%, ±5%, ±4%, ±3%, ±2%, ±1%, ±0.5%, ±0.2%, ±0.1%, ±0.05%, or smaller, as well as the recited value itself Thus, by way of example, “about 10” should be understood to mean “10” and a range no larger than “9-11”. For clarity, as used herein, designation of a range of values includes all integers within or defining the range, and all subranges defined by integers within the range.

As used herein, the term “and/or” refers to and encompasses any and all possible combinations of one or more of the associated listed items, as well as the lack of combinations when interpreted in the alternative (“or”).

As used herein, term “or” refers to any one member of a particular list and also includes any combination of members of that list.

The singular forms of the articles “a,” “an,” and “the” include plural references unless the context clearly dictates otherwise. For example, the term “a pest” or “at least one pest” can include a plurality of proteins, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of′ shall mean excluding more than trace elements of other ingredients for claimed compositions and substantial method steps. Examples and implementations defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation. For example, where the disclosure describes a field, crop, or farm without a “pest” or a “pathogen”, this is also intended to provide antecedent basis for a negative limitation.

Statistically significant means p≤0.05.

### DETAILED DESCRIPTION

### I. Overview

All of the functionalities described in connection with one embodiment of the methods, compositions, or formulations described herein are intended to be applicable to the additional embodiments of the methods, compositions, or formulations described herein except where expressly stated or where the feature or function is incompatible with the additional embodiments. For example, where a given feature or function of component is expressly described in connection with one embodiment but not expressly mentioned in connection with an alternative embodiment, it should be understood that the feature or component may be deployed, utilized, or implemented in connection with the alternative embodiment unless the feature or component is incompatible with the alternative embodiment.

The global economic impact of agricultural pests is significant. For instance, the United States Department of Agriculture (USDA) and the Animal and Plant Health Inspection Service (APHIS) estimates that crop losses to pests and vector born plant pathogens amounts to hundreds of billions of dollars in losses annually. With increasing climate change, deforestation and intercontinental transport of crops, this has been increasing every year. In addition, food security continues to emerge as a major concern as the global population is expected to hit 10 billion by 18 2050. If the population growth forecast holds, it is estimated that current food production would need to be increased by 50% to satisfy the existing demand. There is immense pressure felt among the global agricultural and livestock community to meet the increased demand for food production. However, the excessive use of pesticides has also led to proliferation and selection of resistance genes in pest & pathogens. There is a pressing need for organic, sustainable ways of pest management and mitigation.

In many aspects, the systems described herein provide a measure of a risk of crop failure or damage, a risk of a pest infestation, a risk of a depletion of organic matter, a risk of a soil mineralization, a risk of depletion of beneficial microbiota from the soil or from within the crop from inputs received from sets of quantitative date, e.g., nucleic acid sequencing data. Provided herein are processes and systems for identifying and monitoring health risks (e.g. existing and potential threats) in a variety of environments, particularly agricultural units and its crops, fields, and farms. The present disclosure provides earlier alerts of threat detection to farmers, which in turn support appropriate remedial measures, e.g., removal of infected crops; quarantine of select areas of the farm from affecting the rest of the farm; treatment of select areas with, e.g., the correct and effective pesticides as opposed to indiscriminate use; or another suitable measure. The processes of the disclosure provide granular information to stakeholder supporting precision pest control strategies, including, e.g., use of biocontrol agents like other insects or beneficial pathogens, which are more sustainable, more environment-friendly and less prone to developing resistance in insects or the plant pathogens. In some aspects, the processes of the disclosure identify various microbes, arthropods, and pests directly without requiring cell culture, thus providing a more holistic perspective of all the pests and pathogens in a crop, field, or farm. The processes of the disclosure can provide an actionable understanding of, e.g., the health of a soil in an agricultural field, and provide actionable measures to correct it. This provides a significant improvement from current state-of-the-art techniques where only lab-culturable microbes are detected and identified.

In some aspects, the processes of the disclosure also provide strategies for insect identification and pathogen identification using DNA metabarcoding and universal markers, such as 16SrRNA, ITS, and COI biomarkers. In some aspects, the process of the disclosure provides systems trained to apply triangulation of the data, e.g., by combining data from different potential sources of crop and field casualties (insects, pathogens and affected plant parts) and triangulating the top causal agents and mechanistic pathways towards root-causes. This allows the disclosed systems to predict emerging threats to the field.

Additional details are provided in the following sections.

II. Screening an Agricultural Unit for a plurality of Quantitative Parameters

In some aspects, the disclosure describes a process for screening an agricultural unit for a presence of a taxa, and/or for a risk assessment of a health risk to the agricultural unit. When screening an agricultural unit for potential health risks, one or more processes of the disclosure can be used to assess potential hazards related to, e.g., soil quality, crop cultivation, rhizome, and exposure pathways. A system of the disclosure may have one or more modules for assessing a plant health risk, a soil health risk, a water (run-off) health risk, and/or a pest infestation risk.

In many instances, the process requires inputs from sets of quantitative data (e.g., a plurality of nucleic acid sequences extracted from samples collected from the agricultural unit. The processes described herein applies a highly customized computer program model trained to identify pattern(s) from a plurality of features from the sets of quantitative data e.g., a plurality of nucleic acid sequences). In many instances, a system of the disclosure access a database of highly curated sequences (e.g., nucleic acid sequences of one or more taxa that selected for a threshold of sequencing quality or sequencing coverage). The system then outputs the pattern that is predictive of the health risk to the agricultural unit based on the screening from said plurality of features.

Nucleic Acid Sequences Extracted from the Agricultural Unit

A system of the disclosure can be trained to screen a plurality of quantitative features from the agricultural unit for a pattern that identifies the health risk to the agricultural unit. In some implementations, a system of the disclosure uses input that are directly derived from physical samples of the agricultural unit, e.g., nucleic acid sequences extracted from microbes, arthropods and nematodes present in soil (e.g., rhizome), crops (e.g., leaves/bark), water present in the irrigation and agricultural run-off water (e.g., water-borne pathogens), or dwellings (e.g., barn) of an agricultural unit. The nucleic acid sequences can be collected in any manner, but it is contemplated that in many instances they will be derived directly from a sample or collected from a “trap.” The disclosure contemplates a system trained to recognize a plurality of distinct nucleic acid sequences, and to associate the plurality of distinct nucleic acid sequences with a health risk.

Sequencing-based tests are able to capture information from such nucleic acids, and to provide far more granularity and comprehensive detection of pathogens and arthropods than targeted assays like qPCR or RPA or LAMP, on their own. First and foremost, sequencing tests can detect a new pest, e.g., a pathogen or insect not previously detected in the entire geographical area. Second, pest detection, in particular arthropod detection through sequencing-based approaches is far superior compared to traditional methods relying on microscopic examination of morphology of the insects. The traditional methods are cumbersome, time-consuming, not very reproducible, require specific expertise. Further, many traditional approaches cannot detect different metamorphic stages of the insects such as eggs, larvae and nymph, whereas sequencing-based tech does not have these limitations. The present processes and systems are able to distinguish different metamorphic stages of various based on the plurality of nucleic acids identified.

In many aspects, a system of the disclosure contemplates an analysis of a plurality of nucleic acids extracted from a physical sample of the agricultural unit. Such processing May comprise isolating a plurality of nucleic acids from the physical sample (e.g., soil, leaf, bark, content removed from “trap”, or another sample) and processing the plurality of nucleic acids for sequencing. In some implementations the nucleic acids can be enriched via primer-based target amplification prior to sequencing. In some implementations, a set of primers utilized in primer-based target amplification comprises markers for, e.g., insecticide resistance, antibiotic, and/or anti-fungal resistance marker.

In many aspects, the plurality of nucleic acid sequences extracted from the agricultural unit comprises a plurality of nucleic acid sequences extracted from at least one arthropod, at least one nematode, at least one bacterium, at least one fungus/mold/yeast, at least one protozoa, at least one archaea, and/or at least one virus.

It is contemplated that the computer program model identifies a taxa in the agricultural unit based on a plurality of unique sequences that the disclosure has annotated. In some aspects, the computer program model identifies a taxa in the agricultural unit based on a plurality of chromosomal or mitochondrial metabarcode nucleic acid sequences selected from the group comprising Cytochrome Oxidase I, Cytochrome b, 16S rRNA, ITS in the plurality of nucleic acid sequences extracted from the at least one arthropod. In some aspects the computer program model identifies a taxa in the agricultural unit based on a plurality of chromosomal or mitochondrial metabarcode nucleic acid sequences selected from the group comprising Cytochrome Oxidase I, Cytochrome b, 18S small subunit rRNA, and 28S large subunit rRNA in the plurality of nucleic acid sequences extracted from the at least one nematode. In some aspects, the computer program model identifies a taxa in the agricultural unit based on a plurality of chromosomal or mitochondrial metabarcode nucleic acid sequences selected from the group comprising 16S rRNA, ITS in the plurality of nucleic acid sequences extracted from the at least one bacterium or archaea. In some aspects, the computer program model identifies a taxa in the agricultural unit based on a plurality of chromosomal or mitochondrial metabarcode nucleic acid sequences selected from the group comprising 16S rRNA and ITS in the plurality of nucleic acid sequences extracted from the at least one fungus. In some aspects, the computer program model identifies a taxa in the agricultural unit based on a plurality of chromosomal or mitochondrial metabarcode nucleic acid sequences selected from the group comprising 16S rRNA, 18S rRNA in the plurality of nucleic acid sequences extracted from the at least one protozoa. In some aspects, the computer program model identifies a virus in the agricultural unit based on targeted amplicon sequencing assay.

### Target Enrichment

In some instances, it may be desirable to test for the presence of particular pests in a farm, crop, or field. In such instances, a variety of enrichment methods may be considered, such as amplification-based enrichment assays such as PCR, RPA, NASBA, LAMP, TMA, MDA, RCA, and/or HDA, hybridization-based enrichment assays (solid-phase like microarray or bead-attached or liquid-phase), protein-based enrichment assays (antigen-antibody interactions), aptamer-based capture assays, molecular inversion probes based assays. The extracted nucleic acids can be enriched via hybridization-based target amplification using a panel of, e.g., capture probes selected from the group comprising of: DNA capture probes, ssDNA capture probes, and RNA capture probes. In some implementations, the hybridization-based target amplification is a solid-phase target capture assay, a bead-capture assay, or a liquid-phase capture assay.

The disclosure contemplates a variety of mechanistically distinct strategies for enriching by amplifying a target nucleic acid. Such strategies are contemplated as optional strategies to enrich inputs provided into the system, e.g., inputs that provide a measure of pesticide resistance markers, presence of a particular taxa, developmental stage of a taxa larvae, nymph, eggs, and mature taxa., genomic identity of a crop. In some aspects, the disclosure contemplates a process(es) for pest monitoring and control, and kits for pest monitoring and control that comprise reagents for a polymerase chain reaction (PCR), a reverse-transcription polymerase chain reaction (RT-PCR), a recombinase polymerase amplification (RP A) reaction, a nucleic acid sequence based amplification (NASBA) reaction, a loop mediated isothermal amplification (LAMP) reaction, a transcription mediated amplification (TMA) reaction, a multiple displacement amplification (MDA) reaction, a rolling circle amplification (RCA) reaction, a helicase dependent amplification (HDA) reaction, a strand displacement amplification (SDA) reaction, a nicking and extension amplification reaction (NEAR), an exponential amplification reaction (EXPAR), or a hybridization chain reaction (HCR). In some instances, such reagents may include pan primers for detecting metabarcode regions of arthropods, eukaryotes, fungus, or bacteria. In other instances, such reagents may include targeted primers for detection of such pests.

In some aspects, the disclosure contemplates a process(es) for pest monitoring and control, and kits for pest monitoring and control that comprise reagents for a nucleic acid amplification reaction. In some instances, the nucleic acid amplification reaction is a polymerase chain reaction (PCR), e.g., an RT-PCR (Reverse Transcription Polymerase Chain Reaction). Amplification of nucleic acid sequences by PCR is described in U.S. Pat. Nos. 4,683,195, 4,683,202, and 4,965,188. PCR is now well known in the art and has been described extensively 7 in the scientific literature. See PCR Applications, ((1999) Innis et al., eds., Academic Press, San Diego), PCR Strategies, ((1995) Innis et al., eds., Academic Press, San Diego); PCR Protocols, ((1990) Innis et al., eds., Academic Press, San Diego), and PCR Technology, ((1989) Erlich, ed., Stockton Press, New York). A “real-time” PCR assay is able to simultaneously amplify and detect and/or quantify the starting amount of the target sequence. The basic TaqMan real-time PCR assay using the 5′-to-3′ nuclease activity of the DNA polymerase is described in Holland et al., (1991) Proc. Natl. Acad. Sci. 88:7276-7280 and U.S. Pat. No. 5,210,015. A real-time PCR without the nuclease activity (a nuclease-free assay) has been described in U.S. Patent Publication No. 20100143901Al. The use of fluorescent probes in realtime PCR is described in U.S. Pat. No. 5,538,848. A rapid one-step reverse transcriptase PCR (RT-PCR) is described in U.S. Pat. No. 8,119,353B2. In some instances, such PCT, RT-PCT, or real-time PCR reagents may include pan primers for detecting metabarcode regions of arthropods, eukaryotes, fungus, or bacteria. In other instances, such reagents may include targeted primers for detection of such pests.

In some configurations the nucleic acid amplification enrichment assay is a nucleic acid sequence based amplification (NASBA) assay for producing multiple copies of single stranded RNA NASBS is a primer-dependent technology that can be used for the continuous amplification of nucleic acids in a single mixture at one or two temperatures. Amplification of nucleic acid sequences by NASBS is described in e.g., Deiman, Birgit; van Aarle, Pierre; Sillekens, Peter (2002). “Characteristics and Applications of Nucleic Acid Sequence-Based Amplification (NASBA)”. Molecular Biotechnology. 20 (2): 163-180; Malek, L.; Sooknanan, R.; Compton, J. (1994). Nucleic acid sequence-based amplification (NASBA). Methods in Molecular Biology. Vol. 28. pp. 253-260; Compton, J (1991). “Nucleic acid sequence-based amplification”. Nature. 350 (6313): 91-2. In some instances, such NASBA reagents may include pan primers for detecting metabarcode regions of arthropods, eukaryotes, fungus, or bacteria. In other instances, such reagents may include targeted primers for detection of such pests. In some configurations the nucleic acid amplification enrichment assay is a loop mediated isothermal amplification (LAMP) assay. LAMP approaches deploy multiple primers targeting several regions of the target DNA, thus resulting in high amplification efficiency of a target nucleic acid. LAMP can amplify DNA at a constant temperature, typically between 60° C. to 65° C. Amplification of nucleic acid sequences by LAMP is described in U.S. Patent No. 8 6,410,278 and extensively in the literature. See, e.g., Notomi T, Okayama H, Masubuchi H, Yonekawa T, Watanabe K, Amino N, Hase T (2000). “Loop-mediated isothermal amplification of DNA”. Nucleic Acids Res. 28 (12): 63e-63; Shirshikov, Fedor V.; Pekov, Yuri A.; Miroshnikov, Konstantin A. (2019-04-26). “MorphoCatcher: a multiple-alignment based web tool for target selection and designing taxon-specific primers in the loop-mediated isothermal amplification method”. PeerJ. 7: e6801; Mori Y, Kitao M, Tomita N, Notomi T (2004). “Real-time turbidimetry of LAMP reaction for quantifying template DNA”. J. Biochem. Biophys. Methods. 59 (2): 145-57; Calvert, Amanda E.; Biggerstaff, Brad J.; Tanner, Nathan A.; Lauterbach, Molly; Lanciotti, Robert S. (2017). “Rapid colorimetric detection of Zika virus from serum and urine specimens by reverse transcription loop-mediated isothermal amplification (RT-LAMP)”. PLOS ONE. 12 (9): e0185340. In some instances, such LAMP reagents May include pan primers for detecting metabarcode regions of arthropods, eukaryotes, fungus, or bacteria. In other instances, such reagents may include targeted primers for detection of such pests.

In some configurations the nucleic acid amplification reaction is a transcription mediated amplification (TMA) reaction, an isothermal amplification system utilizing two enzymes, RNA polymerase and reverse transcriptase. Amplification of nucleic acid sequences by TMA is described in, e.g., Daniel L. Kacian, Timothy J. Fultz: Nucleic acid sequence amplification methods. In: Biotechnology Advances 1995, 13.3, S. 569-569. In some instances, such TMA reagents may include pan primers for detecting metabarcode regions of arthropods, eukaryotes, fungus, or bacteria. In other instances, such reagents may include targeted primers for detection of such pests.

In some configurations the nucleic acid amplification reaction is a strand displacement amplification (SDA) reaction or a multiple displacement amplification (MDA) reaction. SDA and MDA are an isothermal amplification technique based upon the ability of a nicking enzyme, e.g., HincII, to nick the unmodified strand of strand (e.g., a hemiphosphorothioate form of Hincll recognition site), and the ability of exonuclease deficient klenow (exo-klenow) to extend the 4 3′-end at the nick and displace the downstream DNA strand. Exponential amplification results from coupling sense and antisense reactions in which strands displaced from a sense reaction serve as target for an antisense reaction and vice versa. SDA is now well known in the art. See, e.g., G. T. Walker, M. C. Little, J. G. Nadeau and D. D. Shank (1992) Proc. Natl. Acad. Sci 89, 392-396; Spits; Le Caignec, C; De Rycke, M; Van Haute, L; Van Steirteghem, A; Liebaers, I; 9 Sermon, K (2006). “Whole-genome multiple displacement amplification from single cells”. Nature Protocols. 1 (4): 1965-70. In some instances, such SDA or MDA reagents may include pan primers for detecting metabarcode regions of arthropods, eukaryotes, fungus, or bacteria. In other instances, such reagents may include targeted primers for detection of such pests.

In some configurations the nucleic acid amplification reaction is a rolling circle amplification (RCA) reaction. RCA is an isothermal nucleic acid amplification technique where the polymerase continuously adds single nucleotides to a primer annealed to a circular template which results in a long concatemer ssDNA that contains tens to hundreds of tandem repeats (complementary to the circular template). RCA is now well known in the art and has been described extensively in the scientific literature. See, e.g., Ali, M. Monsur; Li, Feng; Zhang, Zhiqing; Zhang, Kaixiang; Kang, Dong-Ku; Ankrum, James A; Le, X. Chris; Zhao, Weian (2014). “Rolling circle amplification: a versatile tool for chemical biology, materials science and medicine”. Chemical Society Reviews. 43 (10): 3324-41; Lizardi, Paul M.; Huang, Xiaohua; Zhu, Zhengrong; Bray-Ward, Patricia; Thomas, David C.; Ward, David C. (July 1998). “Mutation detection and single-molecule counting using isothermal rolling-circle amplification”. Nature Genetics. 19 (3): 225-232. In some instances, such RCA reagents may include pan primers for detecting metabarcode regions of arthropods, eukaryotes, fungus, or bacteria. In other instances, such reagents may include targeted primers for detection of such pests.

In some configurations the nucleic acid amplification reaction is a Nicking Enzyme Amplification Reaction (NEAR). NEAR is isothermal, replicating DNA at a constant temperature using a polymerase (and nicking enzyme) to exponentially amplify the DNA at a temperature range of 55° C. to 59° C. NEAR has been described in the literature in U.S. Pat. Nos. 6,191,267, 6,660,475. NEAR is further described in the art in Biochemistry. 2008 September 1 23;47 (38): 9987-99. In some instances, such NEAR reagents may include pan primers for detecting metabarcode regions of arthropods, eukaryotes, fungus, or bacteria. In other instances, such reagents may include targeted primers for detection of such pests.

In some configurations the nucleic acid amplification reaction is a helicase dependent amplification (HDA) reaction. HDA utilizes a DNA helicase to generate single-stranded templates for primer hybridization and subsequent primer extension by a DNA polymerase. HDA has been described in the literature in Saiki R K, et al. (1988). “Primer-directed enzymatic amplification of DNA with a thermostable DNA polymerase”. Science. 239 (4839): 487-491; and Kornberg A, Baker T (1992). DNA Replication, 2nd edn. WH Freeman and Company: New York. In some instances, such HDA reagents may include pan primers for detecting metabarcode regions of arthropods, eukaryotes, fungus, or bacteria. In other instances, such reagents may include targeted primers for detection of such pests.

In some configurations the nucleic acid amplification reaction is a hybridization chain reaction (HCR). HCR provides multiplexed, isothermal, enzyme-free, molecular signal amplification in diverse settings. In some instances, such HCR reagents may include pan primers for detecting metabarcode regions of arthropods, eukaryotes, fungus, or bacteria. In other instances, such reagents may include targeted primers for detection of such pests.

The disclosure contemplates a variety of mechanistically distinct strategies for sequencing the plurality of nucleic acids. The sequencing can comprise e.g., Sanger sequencing. The sequencing can comprise next-generation selected from the group consisting of: sequencing-by-synthesis, semiconductor sequencing, avidity sequencing, single-read flow-based sequencing, and sequencing-by-binding. The sequencing can comprise third-generation sequencing such as single molecule real-time sequencing (e.g., SMRT by Pacific Biosciences) or nanopore sequencing (e.g., Oxford Nanopore Technology).

### III. Screening of an Agricultural Unit for a Taxa, or a Health Risk to the Agricultural Unit

All of the functionalities described in connection with the processes, systems, metabarcode sequences described herein are intended to provide a health assessment of an agricultural unit. In many implementations, this comprises an analysis of at least one taxa from a farm, crop, or field sample. The sample can comprise a mixture of arthropod nucleic acids, eukaryotic nucleic acids (e.g., farm animals such as cows, sheep, horses, and others), fungi nucleic acid, viral nucleic acid, and bacterial nucleic acids.

In many aspects, the disclosure provides a system for screening an agricultural unit for a taxa, the system comprising: a receiving module for receiving a plurality of primary nucleic acid sequences extracted from at least one physical sample collected from the agricultural unit; a database comprising a plurality of nucleic acid sequences uniquely associated with a taxa, the database comprising: a plurality of nucleic acid sequences extracted from at least one arthropod; a plurality of nucleic acid sequences extracted from at least one nematode; a plurality of nucleic acid sequences extracted from at least one bacterium; a plurality of nucleic acid sequences extracted from at least one fungus/mold/yeast; a plurality of nucleic acid sequences extracted from at least one protozoa; and/or a plurality of nucleic acid sequences extracted from at least one archaea; a screening module for comparing the plurality of primary nucleic acid sequences received by the receiving module against the plurality of nucleic acid sequences uniquely associated with the taxa in the database; and an output module for outputting a list of taxa present in the at least one sample from the agricultural unit.

In many implementations, the screening of the plurality of features comprises screening the plurality of nucleic acid sequences extracted from a physical sample form the agricultural unit and forming a first list of raw sequence reads. Subsequently, the screening module forms a second list of sequences, e.g., conserved metasequences or sequences amplified by targeted amplification, present in the first list of raw sequence reads. The computer program model is trained to produce a score from the second list of sequences.

In many aspects, the systems described herein provide a measure of a risk of crop failure or damage, a risk of a pest infestation, a risk of a depletion of organic matter, a risk of a soil mineralization, a risk of depletion of beneficial microbiota from the soil or from within the crop. The systems described below are trained to identify such risks from sets of quantitative data.

### Training of a System of the Disclosure:

A database is built associating one or factors affecting the agricultural unit, e.g., the economic impact of different factors on an agricultural unit. The economic impact is measured by reduction or increase in crop yields in a defined unit of time. These factors include abundance of Pests (arthropods, nematodes, viruses, bacteria and fungi), soil & plant microbial diversity in an agricultural unit over a defined farming period. Once the economic impacts of various taxa on different crops/animals in an agricultural unit are clearly curated, an aggregated model to provide a cumulative risk category is developed by using a weighted score method, with the weights decided based on expert knowledge or derived from training data using a machine learning model using supervised learning algorithms such as random forest or gradient boosting. The fully developed machine learning model considers appropriate weights for individual risk factors and is used for predictive analysis of the risk for an agricultural unit. The cumulative risk uses categories: Low risk, Moderate risk, High risk. If the economic impact is low (<10% compared to the previous year) for at least 5 years in the future, the cumulative risk will be deemed low; if the economic impact is >50%, but <100% this year or >10% but <20% for the next 5 years in the future, the cumulative risk will be deemed moderate and if the economic impact is >100% this year or >20% for the next 5 years, the risk will be deemed high.

### IV. Systems and Databases for Screening an Agricultural Unit for a Health Risk to the Agricultural Unit

In some aspects, the disclosure provides a database comprising a plurality of nucleic acid sequences associated with at least one arthropod; a plurality of nucleic acid sequences associated with at least one nematode; a plurality of nucleic acid sequences associated with at least one bacterium; a plurality of nucleic acid sequences associated with at least one fungus/mold/yeast; a plurality of nucleic acid sequences associated with at least one protozoa; a plurality of nucleic acid sequences associated with at least one archaea; and/or a plurality of nucleic acid sequences associated with at least one virus.

In some aspects, systems of the disclosure provide a curated database with greater than 7,100 genera of bacteria and fungi annotated with greater than 41,000 unique sequences. Each unique sequence was first retrieved from the National Center for Biotechnology Information (NCBI) database, which is maintained by the National Institute of Health in the United States. As a first step, a database of the present disclosure was annotated over 41,000 unique sequences, available from the NCBI database, with greater than 7,100 of the aforementioned genera. Unfortunately, a downside of the raw data retrieved from NCBI is that it frequently comprises noisy data due to poor original quality and curation not being strictly enforced. As a second step, a databases of the disclosure was curated with a second filter, which compares the raw data from the NCBI database against the results of another NCBI database: the nucleotide database. This provides a curated, novel database, as of this filing comprising over 41,000 unique sequences associated with metabarcodes. It is contemplated that a screening system and the risk assessment system of the disclosure can incorporate additional unique sequences associated with additional taxa that are curated in a similar manner. In some aspects, the disclosure provides a database of curated sequences (e.g., nucleic acid sequences of one or more taxa that selected for a threshold of sequencing quality or sequencing coverage) annotated over 41,000 unique sequences.

### V. Model Development, Training, Database Development, and Taxa Databases

Organisms can be collected directly from specimens in an agricultural unit and compared to a reference database of DNA sequences with known taxonomic identities. In some instances, microorganism can be collected from “traps” strategically placed in an agricultural unit. 16S and Internal Transcribed Spacer (ITS) ribosomal RNA (rRNA) sequencing, for instance, are common amplicon sequencing methods used to identify and compare bacteria or fungi present within a given sample. The disclosure describes a meticulously created database, comprising metabarcode reference sequences (e.g., COI sequences, internal transcribed spacer (ITS) sequences, and 16S sequences. The database comprises 41,000+unique sequences of 7100+genera of bacteria & fungi and 3.4 Million sequences of 510,000+genera of arthropods (e.g., insects). The disclosure describes process for triangulating such data, identifying pests and species that are commonly found together, and assigning a risk value to the sequences identified. The disclosure applies the triangulation of such distinct nucleic acid metabarcoding sequences as a basis for distinguishing genera and species in samples extracted from crops, fields, and/or farms.

The raw sequencing data generated from sequencer for the plurality of taxa discussed herein were automatically upload to the cloud for storage and analysis. An automatic program monitors the cloud periodically for the presence of new sequences. When new data is found, an automatic analysis is triggered and the bioinformatics pipeline begins the analysis. The bioinformatic pipeline described herein leverages genomic analysis tools-which receive and analyze genomic sequencing data in standard formats. The bioinformatics model provides proprietary read thresholds, phylogenetic clustering, decision trees and a large curated database to produce a fairly comprehensive report that lists all the taxa present in the samples, with species-level resolution for most. A noise threshold based on normalized read percentage for each sample and read quality filters any noise and cross contamination from other samples in a run. The negative control that is processed and sequenced alongside all the samples provides a measure of noise in a sequencing run.

In some aspects, the disclosure provides systems and process for screening nucleic acid sequences in a samples, e.g. a farm sample and identifying Taxa present therein. In other aspects, the disclosure provides systems and process for providing a risk assessment to a farm based on the identification of one or more nucleic acid sequences in a sample from the farm sample.

In some aspects, a system of the disclosure starts an analysis with a bioinformatic analysis step for assessing the quality of the sequences produced and removing the sequencing adapters from the sequencing reads, for example, one method for doing this applies a tool named cutadapt. Subsequently, primers will be trimmed from, e.g. each individual sequencing read, in the plurality of sequencing reads obtained. The left forward and reverse sequencing reads are then merged with bioinformatics tools, e.g., a suitable tool is SeqPrep, which allows overlap between the bidirectional reads to be 15-20 bp. Subsequently, a clustering software tool is applied. A suitable clustering software tool can be, e.g., Vsearch will is applied for clustering for those sequencing reads that passed a set threshold selected in the model. In many instances, a screening system and a risk assessment system of the disclosure establish a threshold for sequencing reads that requires identity of 99% of greater to a reference database. It is contemplated that the parentage identity can be adjusted, e.g., percentage identity greater than 85%, 86%, 87%, 88%, 89%, 90%, 91%, 92%, 93%

Representative sequences for a plurality, a majority, or for each cluster created as described above are compared to a curated taxonomy database (further described herein) using the NCBI program called Blast. The curation of the database has provided an unprecedent level of resolution to the systems and the process of the disclosure. For the screening and risk assessment systems described herein alignments with less than 80 in length or identity that were less than 90% are ignored in final report. In many instances, the top hit with highest bit score is reported for each cluster, which is also referred herein as operational taxonomic unit (OTU). It is contemplated that up to 2, 3, 4, 5, 6, 7, 8, 9, or 10 top hits may be reported in some instances. Subsequently, the clusters or OTUs with the same taxonomy are instructed to be merged in the final report. Sometimes, for additional validation, the reported sequences for each of the OTUs are mapped against the NCBI maintained NT database. In such instances, a polishing logic based on the percentage of the sequence that is identical to an entry in either of the database, length of the sequence queried, number of evidences supporting a particular taxonomy in the database and whether the entry in the database is generated from a complete genome or a partial genome of the organism are all factors that would be used to resolve conflicts between the different mapping results and a final result combining the mapping against custom-curated database and NCBI NT database would be reported.

Database Development for Metabarcode Annotation of Sequences from 7100+Genera

A first exemplary database was developed with over 41,000 nucleic acid sequences derived from the NCBI database. As part of this effort, Applicant analyzed each of the 41,000 sequences from a plurality of eukaryote, arthropod, fungi and bacterial taxa and annotated specific sequences for COI, 16S, 18S, and/or ITS regions for each of the plurality of eukaryote, arthropod, fungi and bacterial taxa. Further, to remove noise from the background and continue to curate the original data, Applicant aligned each of the annotated specific sequences for COI, 16S, 18S, and/or ITS regions for each of the plurality of eukaryote, arthropod, fungi and bacterial taxa against a second nucleic acid database. This second level of polishing (or filtering) was effectuated by cross-checking the final results against another NCBI database, the nucleotide database. This provided a highly curated database with taxa annotation for COI, 16S, 18S, and/or ITS regions and removal of background noise. The process provided a novel database with curated metabarcode sequences associated with a plurality of eukaryote, arthropod, fungi and bacterial taxa.

### Model Development

The raw sequencing data generated from sequencer is upload to the cloud for storage and analysis using a model. The analysis starts by filtering the sequences based on specific QC criteria for the sequencer used. Then, the sequencing adapter was removed using a tool, in this specific instance a tool named cutadapt although different tools with similar functionality are contemplated. Subsequently, primer sequences were trimmed from each individual read. The left forward and reverse sequencing reads were merged using the model with overlap as 15 bp. A tool called Vsearch was used to performe the clustering for those passed reads using identity 99%, although similar tools have been contemplated. The representative sequences of each cluster were compared with the curated taxonomy database using NCBI BLAST. Alignments with less than 80 in length or identity less than 90% were ignored in final report. Further, the top hit with highest bitscore was reported for each cluster. The clusters from same taxonomy were merged in the final report. In addition, the reported sequences were also mapped using BLAST against the NCBI nucleotide (NT) database. This additional mapping report fine tunned the report obtained from the custom curated database. The model described herein provides a higher resolution as compared to NCBI database.

Training and Validation and integration of the wet lab sequencing method and the bioinformatic method:

The wet lab or microbial part of a system of the invention is designed to screen all microbes present in a sample. An exemplary system was trained and validated using multiple mock microbial mixes or genomic DNA mixes publicly available from different sources, as further discussed below. These microbial mixes had been previously characterized and validated by the source organizations. Exemplary mixes (A) and (B) were used for training the bioinformatic analysis to determine the thresholds for % of sequence reads in a run, % of sequence identity and length thresholds for query sequences. Upon training and generating the thresholds, the analysis was validated using a third mix, referred herein as mix (C).
A) A DNA mix containing the genomic DNA from 10 different fungi, all at the same concentration, named Mycobiome Genomic DNA mix (MSA-1010), a non-limiting example of which is the one sold by ATCC. The expected composition of this mix 17 based on genomic DNA was: 10.0% Aspergillus fumigatus (ATCC MYA-4609D-5), 10.0% Cryptococcus neoformans var. grubli (ATCC 208821D-2), 10.0% 19 Trichophyton interdigitale (ATCC 9533D-5), 10.0% Penicillium chrysogenum (ATCC 10106D-5), 10.0% Fusarium keratoplasticum (ATCC 36031D-5), 10.0% Candida albicans (ATCC 10231D-5), 10.0% Nakaseomyces glabratus (ATCC 2001D-5), 10.0% Malassezia globosa (ATCC MYA-4612D-5), 10.0% Saccharomyces cerevisiae (ATCC 201390D-5), 10.0% Cutaneotrichosporon dermatis (ATCC 204094D-5)
B) A DNA mix containing the genomic DNA from 20 different fungi, all at the same concentration, named Bacterial Genomic DNA mix (MSA-1002) from ATCC. The expected composition based on genomic DNA: 5% Acinetobacter baumannii (ATCC 17978), 5% Bacillus pacificus (ATCC 10987), 5% Phocaeicola vulgatus (ATCC 8482), 5% Bifidobacterium adolescentis (ATCC 15703), 5% Clostridium beijerinckii (ATCC 35702), 5% Cutibacterium acnes (ATCC 11828), 5% Deinococcus radiodurans (ATCC BAA-816), 5% Enterococcus faecalis (ATCC 47077), 5% Escherichia coli (ATCC 700926), 5% Helicobacter pylori (ATCC 700392), 5% Lactobacillus gasseri (ATCC 33323), 5% Neisseria meningitidis (ATCC BAA-335), 5% Porphyromonas gingivalis (ATCC 33277), 5% Pseudomonas paraeruginosa (ATCC 9027), 5% Cereibacter sphaeroides (ATCC 17029), 5% Schaalia odontolytica (ATCC 17982), 5% Staphylococcus aureus (ATCC BAA-1556), 5% Staphylococcus epidermidis (ATCC 12228), 5% Streptococcus agalactiae (ATCC BAA-611), 5% Streptococcus mutans (ATCC 700610).
9 C) A mock community of 8 bacteria and 2 fungi in the form of inactivated organisms at different concentrations (D6300), in this exemplary instance a mix commercialy available from Zymo Research. 11

For all the training and validation process, the different mock microbial mixes were processed through the wet lab workflow first to result in raw DNA sequences as follows. The DNA from the microbial mixes were first extracted using the ZymoBIOMICS DNA Miniprep kit (D4300). The extracted DNA was then taken through the target amplification step where the 16S and ITS regions of the different microbial DNA in the mix were amplified. Then the amplification products from each of the samples were attached to sample-specific DNA barcodes and sequencing adapters through another PCR amplification. Then the resultant libraries for each of the samples were cleaned using a bead cleanup process to remove excess adapters, primer dimers, etc. and the resultant cleaned up libraries from each of the samples were quantified and pooled together to form a single sequencing-ready library. This was then sequenced on a MiSeq sequencer using a 2×300 bp v2 sequencing kit from Illumina.

The validation results of the training are shown below:

TABLE 1
A) Validation A (MSA 1010):

| | | % | % Exp. | Correct |
|---|---|---|---|---|
| Call out | Reads | Abundance | Abundance | Guide |
| Aspergillus_{—}fumigatus | 1600 | 2.5% | 10% | Yes |
| Candida | 16748 | 26.1% | 20% | Yes |
| Cryptococcus | 9241.081997 | 14.4% | 10% | Yes |
| neoformans | | | | |
| Fusarium | 9630 | 15.0% | 10% | Yes |
| Penicillium | 1945.438426 | 3.0% | 10% | Yes |
| Saccharomyces | 9498.092851 | 14.8% | 10% | Yes |
| Trichophyton | 1730 | 2.7% | 10% | Yes |
| Cutaneotrichosporon | 13655 | 21.3% | 10% | Yes |
| Malassezia globosa | 0 | 0.0% | 10% | No |
| | | | True Positive | 9/10 |
| | | | True Negative | 1/10 |

TABLE 2
B) Validation B (MSA 1002);

| | | | % Exp. | |
|---|---|---|---|---|
| Call out | Reads | % Abundance | Abundance | Correct Guide |
| Staphylococcus | 7207.688633 | 10.2% | 10% | Yes |
| Streptococcus | 10572.33633 | 15.0% | 10% | Yes |
| Acinetobacter_{—}baumanii | 4218.760176 | 6.0% | 5% | Yes |
| Bacillus | 5366.548015 | 7.6% | 5% | Yes |
| Bifidobacterium | 1966.405211 | 2.8% | 5% | Yes |
| Cereibacter sphaeroides | 529.8754789 | 0.7% | 5% | Yes |
| Clostridium | 9078.342832 | 12.8% | 5% | Yes |
| Escherichia | 1363.000893 | 1.9% | 5% | Yes |
| Lactobacillus | 5309.318282 | 7.5% | 5% | Yes |
| Neisseria | 3863.199375 | 5.5% | 5% | Yes |
| Phocaeicola | 7254.497934 | 10.3% | 5% | Yes |
| Pseudomonas | 1678.020384 | 2.4% | 5% | Yes |
| Porphyromonas | 3752 | 5.3% | 5% | Yes |
| gingivalis | | | | |
| Helicobacter pylori | 1835.388292 | 2.6% | 5% | Yes |
| Actinomyces | 171.8333333 | 0.2% | 5% | Yes |
| Deinococcus | 1168.244726 | 1.7% | 5% | Yes |
| Enterococcus faecalis | 2344.502775 | 3.3% | 5% | Yes |
| Cutibacterium acnes | 0 | 0.0% | 5% | No |
| | | | True Positive | 16/17 |
| | | | True Negative | 1/17 |

TABLE 3
C) Zymo D6300:

| | | | | |
|---|---|---|---|---|
| | | % | % Exp. | |
| Call out | Reads | Abundance | Abundance | Correct Guide |

| | | | |
|---|---|---|---|
| Bacillus | 15% | 12% | Yes |
| Enterococcus_{—}faecalis | 8.8% | 12% | Yes |
| Escherichia | 5.9% | 12% | Yes |
| Lactobacillus | 20.6% | 12% | Yes |
| Pseudomonas | 3.1% | 12% | Yes |
| aeruginosa | | | |
| Salmonella_{—}enterica | 8.7% | 12% | Yes |
| Staphylococcus | 8.1% | 12% | Yes |
| Listeria | 15.4% | 12% | Yes |
| monocytogenes | | | |
| Crysptococcus | 2.0% | 2% | Yes |
| neoformans | | | |
| Saccharomyces | 3.1% | 2% | Yes |
| | True Positive | 10/10 | |
| | True Negative | 0/10 | |

### Taxa Database

Approximately one-quarter of insect species exhibit high intraspecific genetic variation (>3%) of the cytochrome c oxidase subunit I (COI), a mitochondrial gene. In some aspects, the present disclosure describes a curated database comprising greater than 3.4 million unique nucleic acid sequences across 510,000+genera of arthropods. As a first step, the disclosure parsed and curated data from the United States National Center for Biotechnology Information (NCBI) into metabarcoding databases. In some aspects, a metabarcoding database of the disclosure was constructed by annotating at least 1,000 unique COI nucleic acid sequences ranging from about 400-700 base pairs, associated with eukaryotes, particularly arthropods and certain genuses/species that can be present in crops, fields, and farms. In some cases a metabarcoding database of the disclosure was created with greater than 1,000 nucleic acid sequences from: Megaselia_sp._BOLD-2016, Bemisia_tabaci, Delia_platura, Smittia_sp._ES12, Entomobrya_nivalis, Limnophyes_sp._14ES, Bactrocera_dorsalis, Cricotopus_sp._BOLD-2016, Orthocladius_sp._BOLD-2016, Apis_mellifera, Echinococcus_granulosus, Psychoda_sp._BOLD-2016, Aedes_albopictus, Limnophyes_sp._BOLD-2016, Balanus_glandula, Aedes_aegypti, Tanytarsus_mendax, Lymantria_dispar, Austrosciara_hyalipennis, Telenomus_sp._BOLD-2016, Scatopsciara_atomaria, Paratanytarsus_laccophilus, Paraphaenocladius_impensus, Gymnometriocnemus_brumalis, Gammarus_fossarum, Spodoptera_frugiperda, Culex_pipiens, Entomobrya_sp._BOLD: ACL6239, Culicoides_obsoletus, Cricotopus_triannulatus, Scathophaga_furcata, Megaselia_arcticae, Pollenia_pediculata, Smittia_sp._BOLD-2016, Forcipomyia_sp._BOLD-2016, Paraphaenocladius_sp._BOLD-2016, Leptocera_erythrocera, Hydrophoria_lancifer, Empoasca_sp._BOLD-2016, Heterotrissocladius_oliveri, Gymnometriocnemus_sp._2_TE-2010, Cameraria_ohridella, Diaphorina_citri, Corynoptera_sp._BOLD-2016, Formica_sp._BOLD-2016, Plutella_xylostella, Dasyhelea_sp._BOLD-2016, Helina_evecta, Entomobrya_atrocincta, Taeniothrips_inconsequens, Culex_quinquefasciatus, Pseudolycoriella_brunnea, Helicoverpa_armigera, Bulinus_tropicus, Cortinicara_gibbosa, Brachionus_calyciflorus, Cylas_formicarius, Cottus_sp._MY-2021, Mycetophila_sp._BOLD-2016, Ixodes_ricinus, Scoparia_biplagialis, Emerita_analoga, Promachocrinus_kerguelensis, Bradysia_sp._BOLD-2016, Zeugodacus_cucurbitae, Limnophyes_sp._BOLD: ABU5525, Phyllobius_oblongus, Myrmica_rubra, Pegomya_sp._BOLD-2016, Dermanyssus_gallinae, Helina_rufitibia, Apis_cerana, Empoasca_luda, Homo_sapiens, Ostrinia_furnacalis, Bactrocera_correcta, Cottus_asper, Limnophyes_minimus, Echinogammarus_sicilianus, Gymnometriocnemus_sp._BOLD-2016, Dinotrema_sp._BOLD-2016, Chironomus_environmental_sample, Megaselia_rufipes, Ochlerotatus_sticticus, Diplonevra_nitidula, Tetragonisca_angustula, Megaselia_lucifrons, Mycetophila_fungorum, Ampullaceana_balthica, Scoparia_penumbralis, Entomobrya_intermedia, Parakiefferiella_sp._BOLD-2016, Pomacea_canaliculata, Fannia_spathiophora, Halyomorpha_halys, Mytilus_galloprovincialis, Boletina_sp._BOLD-2016, Smittia_sp._8ES, Megaselia_diversa, Pieris_rapae, Scaptomyza pallida, Corynoptera_perpusilla, Scatopsciara_sp._BOLD: AAH3920, Siphonaria_lessonii, Phyllocnistis_populiella, Helina_sp._BOLD-2016, Corynoptera_furcata, Rhipicephalus_microplus, Orthocladius_oliveri, Carollia_perspicillata, Lepidocyrtus_paradoxus, Anisakis_pegreffii, Lasius_sp._BOLD-2016, Megaselia_pulcaria_complex_sp._BOLD-2016, Varroa_destructor, Eiseniella_tetraedra, Perna_perna, Aedes_sp._BOLD: AAA7067, Bactrocera_minax, Linckia_laevigata, Corynoptera_trepida, Drosophila_affinis, Aphis_gossypii, Tridacna_crocea, Mycomya_affinis, Tanytarsus_lugens, Bombus_morio, Lycoriella_sp._BOLD-2016, Erythroneura_tricincta, Ablabesmyia_sp._BOLD: AAC8567, Nemorimyza_posticata, Ctenocephalides_felis, Tapinoma_sessile, Prenolepis_imparis, Culex_tritaeniorhynchus, Lygus_lineolaris, Heterotrissocladius_sp._BOLD: AAN5369, Luthrodes_pandava, Macrorchis_ausoba, Parakiefferiella_sp._BOLD: AAI2681, Limnophyes_sp._BOLD: AAN5339, Metcalfa_pruinosa, Micropsectra_subletteorum, Hydrotaea_sp._BOLD: AAG1768, Corynoneura_sp._1ES, Liocarcinus_depurator, Oncomelania_hupensis, Turbinaria_mesenterina, Lasiomma_sp._BOLD-2016, Suillia_convergens, Limnophyes_schnelli, Diaphanosoma_dubium, Eisenia_andrei, Chaitophorus_populifolii, Suillia_nemorum, Paratanytarsus_environmental_sample, Schistocephalus_solidus, Metriocnemus_sp._BOLD: ACM6798, Pleuromamma_abdominalis, Strongyloides_stercoralis, Simulium_sp._BOLD-2016, Ruditapes_philippinarum, Callinectes_sapidus, Periplaneta_americana, Cladotanytarsus_aff._mancus_3TE, Calanus_glacialis, Gallus_gallus, Mytilus_trossulus, Tanytarsus_sp._BOLD-2016, Procladius_dentus, Pyura_chilensis, Baetis_tricaudatus, Corophium_volutator.

In some aspects, a metabarcoding database of the disclosure further comprises additional unique COI nucleic acid sequences associated with eukaryotes, particularly arthropods and certain genuses/species that can be present in crops, fields, and farms. In some aspects, a database of the disclosure was annotated with 500 to 1,000 unique COI sequences from the following arthropod genus: Aedes_sp._BOLD-2016, Appasus_japonicus, Rhynchophorus_ferrugineus, Iteaphila_orchestris, Dicrotendipes_tritomus, Thienemanniella_xena, Tridacna_maxima, Frankliniella_occidentalis, Cordyla_sp._BOLD-2016, Ophioderma_longicauda, Bombus_terrestris, Torymus_sinensis, Limnophyes_sp._BOLD: AAG5426, Chrysotus_sp._BOLD-2016, Aedes_vexans, Excirolana_hirsuticauda, Rotaria_rotatoria, Bradysia_placida, Stenomacrus_sp._jft21, Smittia_sp._23ES, Macrosteles_quadrilineatus, Anisakis_simplex, Semibalanus_balanoides, Pardosa_moesta, Peripsocus_subfasciatus, Isonychia_valida, Haemaphysalis_longicornis, Schistosoma_mansoni, Chironomus_sp._BOLD-2016, Chironomus_riparius, Hyalessa_maculaticollis, Bradysia_trivittata, Limnophyes_natalensis, Reishia_clavigera, Melarhaphe_neritoides, Enoplognatha_ovata, Thysanoessa_raschii, Coenosia_tigrina, Aceria_tosichella, Leptocybe_invasa, Rhipicephalus_sanguineus, Maruca_vitrata, Megaselia_arizonensis, Prionace_glauca, Oreochromis_niloticus, Toxomerus_geminatus, Thrips_tabaci, Rattus_tanezumi, Philygria_obtecta, Drosophila_neotestacea, Limecola_balthica, Aquarius_paludum, Bryotropha_similis, Carcinus_maenas, Hydrobaenus_fusistylus, Pediculus_humanus, Culicoides_scoticus, Dolichopus_plumipes, Culex_torrentium, Lumbricus_terrestris, Telenomus_podisi, Malloewia_abdominalis, Isochnus_sp._BOLD: ACA3052, Smittia_cf._stercoraria_BOLD-2016, Chrysops_sp._BOLD: AAA8870, Neotelphusa_querciella, Mytilus_edulis, Harmonia_axyridis, Rhamphomyia_sp._BOLD-2016, Orchestoidea_tuberculata, Tuta_absoluta, Mothocya_parvostis, Gammarus_roeselii, Brachypogon_sp._BOLD-2016, Toxomerus_marginatus, Fasciola_hepatica, Orchesella_sp._BOLD: AAA6611, Rhagio_mystaceus, Turbinaria_reniformis, Entomobrya_unostrigata, Aporrectodea_caliginosa, Hyperlasion_wasmanni, Drosophila_melanogaster, Hermetia_illucens, Myzus_persicae, Gomphiocephalus_hodgsoni, Monodonta_labio, Cyprinus_carpio, Eucypris_virens, Artibeus_lituratus, Coccinella_septempunctata, Dicrotendipes_sp._BOLD-2016, Littorina_littorea, Entomobrya_sp._BOLD: AAA7249, Artemia_parthenogenetica, Hydroptila_sp._BOLD: AAE1778, Eisenia_nordenskioldi, Neotrypaea_californiensis, Lycoriella_sativae, Empoasca_fabae, Lunella_smaragdus, Philaenus_spumarius, Monopelopia_tenuicalcar, Lasius_neoniger, Maniola_jurtina, Sylvicola_sp._BOLD: AAG1999, Loxosceles_rufescens, Micropsectra_polita, Trichopalpus_punctipes, Patiria_miniata, Metacarcinus_magister, Leptidea_sinapis, Daphnia_magna, Erasmoneura_vulnerata, Psyllobora_vigintimaculata, Thrips_palmi, Limacina_helicina, Dikraneura_mali, Chrysotus_sp._BOLD: AAG9668, Chrysomya_megacephala, Trialeurodes_vaporariorum, Euzetes_globulus, Polydrusus_formosus, Notochthamalus_scabrosus, Liriomyza_trifolii, Pollicipes_polymerus, Chthamalus_malayensis, Botryllus_schlosseri, Eurytemora_affinis, Diolcogaster_sp._Choi107, Tetragnatha_laboriosa, Cerobasis_guestfalica, Heterotrissocladius_sp._BOLD-2016, Coenosia_sp._BOLD-2016, Ceratopsyche_sparna, Chthamalus_challengeri, Brassicogethes_aeneus, Atrichopogon_sp._BOLD-2016, Acanthochitona_rubrolineata, Atta_cephalotes, Cricotopus_sp._BOLD: AAG0996, Osbornellus_auronitens, Heterotrissocladius_changi, Pegoscapus_sp., Cladotanytarsus_pallidus, Culicoides_imicola, Eulaema_atleticana, Cepaea_nemoralis, Cratyna_longispina, Alviniconcha_kojimai, Artemia_franciscana, Corynoptera_melanochaeta, Aphis_craccivora, Dryophytes_japonicus, Cellana_toreuma, Stenomacrus_sp._BOLD-2016, Cricotopus_bicinctus, Macaria_signaria, Leptosciarella_sp._BOLD-2016, Hirudo_verbana, Aedes_japonicus, Euphydryas_aurinia, Macrobrachium_nipponense, Pantala_flavescens, Adoretus_sp._AM-2015, Spilogona_arctica, Phoxinus_lumaireul, Clubiona_canadensis, Bactrocera_zonata, Chironomus_maturus, Contacyphon_variabilis, Valenzuela_flavidus, Artibeus_planirostris, Corynoptera_subtrivialis, Entomobrya_multifasciata, Entomobrya_assuta, Mycetophila_sp._BOLD: AAG4872, Crassostrea_gigas, Erythroneura_rubrella, Lepidocyrtus_cyaneus, Polyommatus_icarus, Neotelphusa_praefixa, Heleobia_ascotanensis, Crassostrea_sikamea, Themisto_libellula, Anopheles_triannulatus, Rhopalosiphum_padi, Corynoptera_cursor, Arctia_plantaginis, Sitophilus_oryzae, Anaspis_rufa, Oratosquilla_oratoria, Hydropsyche_betteni, Doratura_stylata, Sphyrna_lewini, Bibio_xanthopus, Ophraella_communa, Artibeus_obscurus, Asellus_aquaticus, Cryptocercus_punctulatus, Formica_neorufibarbis, Caprella_scaura, Alviniconcha_boucheti, Lasius_alienus, Reticulitermes_flavipes, Philodromus_rufus, Anopheles_stephensi, Protophormia_terraenovae, Fistulobalanus_albicostatus, Drosophila_suzukii, Thunnus_albacares, Sigmella_schenklingi, Platypalpus_sp._BOLD-2016, Choristoneura_fumiferana, Anoplophora_glabripennis, Aulacigaster_neoleucopeza, Liriomyza_sativae, Bradysia_impatiens, Sorex_cinereus, Limacina_bulimoides, Forcipomyia_sp._6ES, Sabellaria_alveolata, Pocadicnemis_americana, Spodoptera_exigua, Lycoriella_flavipeda, Operophtera_bruceata, Cricotopus_trifascia, Eleutheronema_tetradactylum, Phyllotreta_striolata, Themira_nigricornis, Sitophilus_zeamais, Gammarus_oceanicus, Nanorana_parkeri, Ceraesignum_maximum, Eupha usia_superba, Polyplax_serrata, Lucilia_sericata, Mus_musculus, Rhopobota_naevana, Equus_asinus, Pteronotus_parnellii, Terapon_jarbua, Angiostrongylus_vasorum, Anopheles_sinensis, Nezara_viridula, Mugil_cephalus, Anodonta_anatina, Paratanytarsus_sp._BOLD: AAV6213, Sarcoptes_scabiei, Larimichthys_polyactis, Cricotopus_sp._7ES, Tetranychus_urticae, Ocypode_ceratophthalmus, Stomoxys_calcitrans, Dicrotendipes_environmental_sample, Stictochironomus_sticticus, Paratanytarsus_sp._7TE, Aphis_glycines, Ophionotus_victoriae, Phaonia_consobrina, Sarcophaga_subvicina, Anopheles_plumbeus, Cryptotendipes_darbyi, Astacus_astacus, Pratylenchus_neglectus, Entomobrya_gisini, Trixagus_carinicollis, Scyliorhinus_canicula, Heteromys_pictus, Anopheles_sp., Pediculus_humanus_complex_sp._D_MA-2014, Papilio_machaon, Simulium_sp._BOLD: AAE8840, Creagrura_rogerblancoi, Pseudosciara_sp., Formica_cf._neorufibarbis_2_BOLD-2011, Prosimulium_mixtum, Cricotopus_sp._4ES, Lepetodrilus_elevatus, Vespa_velutina, Channa_striata, Cynomya_cadaverina, Eleotris_fusca, Phlebotomus_argentipes, Liohippelates_bishoppi, Phoxinus_phoxinus, Chironomus_atrella, Ixodes_scapularis, Valenzuela_sp._BOLD-2016, Dissostichus_eleginoides, Acanthaster_planci, Cricotopus_environmental_sample, Cotesia_xylina, Hylemya_sp._BOLD-2016, Euceraphis_papyrifericola, Baetis_rhodani, Orthocladius_sp._BOLD: AAB2644, Nerita_albicilla, Argyrodes_sp._Rodrigues, Simulium_vittatum, Forcipomyia_glauca, Hermodice_carunculata, Anguillicola_crassus, Olcella_sp._BOLD-2016, Bemisia_argentifolii, Chlorops_oryzae, Myrmica_incompleta, Hemiculter_leucisculus, Wuchereria_bancrofti, Anthonomus_eugenii, Platypalpus_sp._BOLD: AAF9772, Lepidocyrtus_lignorum, Drosophila_falleni, Chironomus_athalassicus, Dirofilaria_immitis, Diarsia_dislocata, Nilaparvata_lugens, Ablabesmyia_americana, Culicoides_obsoletus_02_MA-2012, Camponotus_herculeanus, Adelges_cooleyi, Fasciola_gigantica, Macrosiphum_sp._BOLD-2016, Aptinothrips_rufus, Psectrocladius_environmental_sample, Hemigrapsus_oregonensis, Monadenia_fidelis, Callyspongia_vaginalis, Diporeia_hoyi, Salmo_salar, Pimpla_sp._BOLD-2016, Niviventer_confucianus, Smittia_sp._22ES, Orgilus_sp._BOLD-2016, Camptochaeta_delicata, Armadillidium_vulgare, Haemonchus_contortus, Bombus_lucorum, Peromyscus_maniculatus, Leptus_sp._BOLD: ACE1761, Mus_spretus, Cumberlandia_monodonta, Procladius_sp._1ES, Pseudohemiculter_dispar, Metriocnemus_sp._BOLD-2016, Adelges_tsugae, Calliphora_vicina, Tritia_obsoleta, Chaitophorus_populicola, Tetraclita_serrata, Aphis_spiraecola, Aedes_cinereus, Oecetis_inconspicua, Bathyphantes_pallidus, Alopecosa_aculeata, Tetrix_japonica, Glossina_fuscipes, Lates_calcarifer, and/or Brachionus_plicatilis.

In some aspects, a metabarcoding database of the disclosure further comprises additional unique COI nucleic acid sequences associated with eukaryotes, particularly arthropods and certain genuses/species that can be present in crops, fields, and farms. In some aspects, a database of the disclosure was annotated with 200 to 499 unique COI sequences from the following arthropod genus: Aulacigaster_neoleucopeza, Liriomyza_sativae, Bradysia_impatiens, Sorex_cinereus, Limacina_bulimoides, Forcipomyia_sp._6ES, Sabellaria_alveolata, Pocadicnemis_americana, Spodoptera_exigua, Lycoriella_flavipeda, Operophtera_bruceata, Cricotopus_trifascia, Eleutheronema_tetradactylum, Phyllotreta_striolata, Themira_nigricornis, Sitophilus_zeamais, Gammarus_oceanicus, Nanorana_parkeri, Ceraesignum_maximum, Euphausia_superba, Polyplax_serrata, Lucilia_sericata, Mus_musculus, Rhopobota_naevana, Equus_asinus, Pteronotus_parnellii, Terapon_jarbua, Angiostrongylus_vasorum, Anopheles_sinensis, Nezara_viridula, Mugil_cephalus, Anodonta_anatina, Paratanytarsus_sp._BOLD: AAV6213, Sarcoptes_scabiei, Larimichthys_polyactis, Cricotopus_sp._7ES, Tetranychus_urticae, Ocypode_ceratophthalmus, Stomoxys_calcitrans, Dicrotendipes_environmental_sample, Stictochironomus_sticticus, Paratanytarsus_sp._7TE, Aphis_glycines, Ophionotus_victoriae, Phaonia_consobrina, Sarcophaga_subvicina, Anopheles_plumbeus, Cryptotendipes_darbyi, Astacus_astacus, Pratylenchus_neglectus, Entomobrya_gisini, Trixagus_carinicollis, Scyliorhinus_canicula, Heteromys_pictus, Anopheles_sp., Pediculus_humanus_complex_sp._D_MA-2014, Papilio, machaon Simulium_sp._BOLD: AAE8840, Creagrura_rogerblancoi, Pseudosciara_sp., Formica_cf._neorufibarbis_2_BOLD-2011, Prosimulium_mixtum, Cricotopus_sp._4ES, Lepetodrilus_elevatus, Vespa_velutina, Channa_striata, Cynomya_cadaverina, Eleotris_fusca, Phlebotomus_argentipes, Liohippelates_bishoppi, Phoxinus_phoxinus, Chironomus_atrella, Ixodes_scapularis, Valenzuela_sp._BOLD-2016, Dissostichus_eleginoides, Acanthaster_planci, Cricotopus_environmental_sample, Cotesia_xylina, Hylemya_sp._BOLD-2016, Euceraphis_papyrifericola, Baetis_rhodani, Orthocladius_sp._BOLD: AAB2644, Nerita_albicilla, Argyrodes_sp._Rodrigues, Simulium_vittatum, Forcipomyia_glauca, Hermodice_carunculata, Anguillicola_crassus, Olcella_sp._BOLD-2016, Bemisia_argentifolii, Chlorops_oryzae, Myrmica_incompleta, Hemiculter_leucisculus, Wuchereria_bancrofti, Anthonomus_eugenii, Platypalpus_sp._BOLD: AAF9772, Lepidocyrtus_lignorum, Drosophila_falleni, Chironomus_athalassicus, Dirofilaria_immitis, Diarsia_dislocata, Nilaparvata_lugens, Ablabesmyia_americana, Culicoides_obsoletus_02_MA-2012, Camponotus_herculeanus, Adelges_cooleyi, Fasciola_gigantica, Macrosiphum_sp._BOLD-2016, Aptinothrips_rufus, Psectrocladius_environmental_sample, Hemigrapsus_oregonensis, Monadenia_fidelis, Callyspongia_vaginalis, Diporeia_hoyi, Salmo_salar, Pimpla_sp._BOLD-2016, Niviventer_confucianus, Smittia_sp._22ES, Orgilus_sp._BOLD-2016, Camptochaeta_delicata, Armadillidium_vulgare, Haemonchus_contortus, Bombus_lucorum, Peromyscus_maniculatus, Leptus_sp._BOLD: ACE1761, Mus_spretus, Cumberlandia_monodonta, Procladius_sp._1ES, Pseudohemiculter_dispar, Metriocnemus_sp._BOLD-2016, Adelges_tsugae, Calliphora_vicina, Tritia_obsoleta, Chaitophorus_populicola, Tetraclita_serrata, Aphis_spiraecola, Aedes_cinereus, Oecetis_inconspicua, Bathyphantes_pallidus, Alopecosa_aculeata, Tetrix_japonica, Glossina_fuscipes, Lates_calcarifer, Brachionus_plicatilis, Apis_dorsata, Monopterus_albus, Minettia_lupulina, Smittia_edwardsi, Boletina_dissipata, Balclutha_rhenana, Nerita_plicata, Parakiefferiella_cf._fennica_BOLD-2016, Limecola_petalum, Carabus_taedatus, Limnodrilus_hoffmeisteri, Bullacta_exarata, Neotricula_aperta, Asynarchus_lapponicus, Littoraria_subvittata, Rattus_norvegicus, Halocladius_variabilis, Dikerogammarus_haemobaphes, Pristionchus_pacificus, Paratanytarsus_sp._BOLD-2016, Dicrotendipes_modestus, Microhyla_fissipes, Parectopa_albicostella, Misumena_vatia, Bradysia_angustipennis, Hybomitra_illota, Acartia_tonsa, Ophiothrix_fragilis, Pseudolycoriella_sp._BOLD-2016, Bathymodiolus_thermophilus, Lasioglossum_morio, Frankliniella schultzei, Limnophyes sp. BOLD: AAB1809, Scirtothrips dorsalis, Grammonota angusta, Atrina pectinata, Ceracris kiangsu, Octopus cyanea, Homalictus fijiensis, Malthodes pumilus, Dasyopa triangulata, Brachycentrus americanus, Phaonia pallidisquama, Spilogona sororcula, Aricia agestis, Agabus bipustulatus, Dinotrema sp. BOLD: AAA7636, Semisulcospira libertina, Pseudocalanus newmani, Octopus vulgaris, Panulirus homarus, Physella acuta, Cratyna sp. BOLD-2016, Potomida littoralis, Culicoides sp. BOLD-2016, Operophtera brumata, Parastacus pugnax, Tribolium castaneum, Aporrectodea trapezoides, Palingenia longicauda, Austropotamobius pallipes, Apis florea, Laricobius osakensis, Pyramidula pusilla, Campaea perlata, Adoncholaimus daikokuensis, Dolichopus lamellipes, Larimichthys crocea, Helophora insignis, Bradysiopsis vittigera, Diplocolenus abdominalis, Atule mate, Rivellia steyskali, Bubalus bubalis, Lucanus cervus, Aphidius ervi, Kraussillichirus kraussi, Taenia hydatigena, Chthamalus fragilis, Vanessa atalanta, Chironomus cf. decorus BOLD: AAB7030, Tortrix viridana, Trichinella spiralis, Aphis fabae, Laricobius nigrinus, Strongylocentrotus droebachiensis, Mamestra configurata, Gonatocerus sp. BOLD-2016, Sogatella furcifera, Achipteria coleoptrata, Bradysia polonica, Paria fragariae, Thricops sp. BOLD-2016, Sympistis zetterstedtii, Bithynia siamensis, Tanytarsus anderseni, Urechis unicinctus, Wasmannia auropunctata, Tetramorium sp. E BK-2018, Ceratopsyche bronta, Terrilimosina schmitzi, Anopheles albimanus, Merluccius merluccius, Micropsectra nigripila, Lumbricus rubellus, Megaselia sp. BOLD: AAG3266, Psyllobora borealis, Camponotus pennsylvanicus, Afronurus yoshidae, Sepia officinalis, Scathophaga stercoraria, Microvelia douglasi, Glischrochilus sanguinolentus, Lepas anatifera, Corynoptera minima, Stygiopontius lauensis, Caucasotachea vindobonensis, Parisotoma notabilis, Eisenia fetida, Procambarus clarkii, Colossendeis megalonyx, Tetragnatha versicolor, Bradysia vagans, Ochlerotatus communis, Phaonia monticola, Asynarchus montanus, Phalangium opilio, Haloptilus longicornis, Hypera postica, Helina depuncta, Molossus molossus, Pigritia murtfeldtella, Porcellio scaber, Pontoscolex corethrurus complex sp. LI, Rhinophylla pumilio, Heterorhabditis bacteriophora, Phormia regina, Urbanus proteus, Sitodiplosis mosellana, Chironomus sp. BOLD: ACK9793, Penaeus monodon, Cerodontha muscina, Pectinophora gossypiella, Erythroneura sp. BOLD: AAV0161, Coryphaena hippurus, Dictyna major, Illinoia rubicola, Pseudocalanus acuspes, Megaselia citrinella, Gammarus minus, Pinna nobilis, Concholepas concholepas, Scomber scombrus, Limnophyes ninae, Oncorhynchus mykiss, Entomobrya comparata, Corynoneura scutellata, Orthocladius sp. TE13, Diolcogaster sp. Choi106, Sciara hebes, Chelipoda truncata, Anopheles farauti, Myrmecina sp. 2 ITOdet, Petrolisthes elongatus, Ancylomenes pedersoni, Hillia iris, Rhyzopertha dominica, Contacyphon obscurus, Chaitophorus saliniger, Xenostrobus securis, Lottia scabra, Metrocoris sichuanensis, Chlorurus sordidus, Psychoda trinodulosa, Sitobion avenae, Laodelphax striatellus, Hypenodes sp. BOLD: AAA3002, Neoscona arabesca, Carcharhinus falciformis, Boletina sp. BOLD: AAL9139, Tachinidae gen. tachJanzen01 sp. Janzen01, Lambdina fiscellaria, Jassa marmorata, Ranoidea raniformis, Simplicia moorei, Lygus robustus, Marmota himalayana, Dichopygina sp. BOLD-2016, Belyta validicornis, Glyphoglossus yunnanensis, Anastatus orientalis, Labeo rohita, Martes americana, Phyllonorycter issikii, Gerris latiabdominis, Melanophthalma inermis, Eulithis destinata, Cydia pomonella, Pisaster ochraceus, Phaonia sp. BOLD-2016, Limnophyes brachytomus, Nigronia serricornis, Hypnoidus bicolor, Reticunassa festiva, Drosophila sp. BOLD: AAB8851, Parnassius glacialis, Monachoides incarnatus, Pollenia rudis, Anastrepha obliqua, Arianta arbustorum, Ascaris lumbricoides, Eusphalerum pothos, Elliptio dariensis, Hediste diversicolor, Holothuria atra, Choristoneura conflictana, Aname mellosa, Camptochaeta sp. BOLD-2016, Cotesia vestalis, Trichiurus lepturus, Myrmica sp. BOLD-2016, Chironomus sp. BOLD: AAB7030, Chamobates cuspidatus, Melissotarsus insularis, Melanaphis sacchari, Cryptopygus antarcticus, Zeugodacus tau, Clitarchus hookeri, Mytilus californianus, Sillago sihama, Lygus sp. BOLD-2016, Anopheles oryzalimnetes, Paracentrotus lividus, Macrobrachium rosenbergii, Evacanthus sp. BOLD-2016, Brachidontes sp. lineage A CDL-2020, Eukrohnia hamata, Leucinodes orbonalis, Sthenoteuthis oualaniensis, Ifremeria nautilei, Monodonta confusa, Cricotopus infuscatus, Hyalomma dromedarii, Scatella sp. BOLD: AAG2738, Margaritifera margaritifera, Anopheles darlingi, Microplitis varicolor, Bulinus truncatus, Paratanytarsus grimmii, Bugula neritina, Nanorana pleskei, Idiocerus suturalis, Blaps rhynchoptera, Panulirus penicillatus, Polypogon jacchusalis, Hyphantria cunea, Bombus ardens, Willowsia buskii, Nematocharax venustus, Alvinella pompejana, Lottia austrodigitalis, Armigeres subalbatus, Agelenopsis utahana, Lepidocyrtus sp. BOLD: ACM2009, Sepioteuthis lessoniana, Semibalanus cariosus, Penaeus semisulcatus, Squaliobarbus curriculus, Chionaspis pinifoliae, Formica glacialis, Elmis aenea, Geothelphusa dehaani, Symplecta cana, Paralicella caperesca, Sturnira lilium, Garrulax canorus, Tamarixia radiata, Sylvicola fuscatus, Austrovenus stutchburyi, Zaphne borealis, Orcula dolium, Holothuria whitmaei, Platypalpus holosericus, Allolobophora chlorotica, Smittia sp. 16ES, Eriocrania semipurpurella, Oribatula tibialis, Mytilus chilensis, Hyalopterus pruni, Lucilia Caesar, Peromyscus mexicanus, Caenorhabditis japonica, Polyarthra dolichoptera complex sp. UO-2013, Synopeas pennsylvanicum, Chthamalus dalli, Bryophaenocladius sp. 8ES, Eulimnogammarus verrucosus, Nucella ostrina, Cucumaria frondosa, Esox lucius, Ceratitis capitata, Potamopyrgus antipodarum, Apanteles ensiger, Lasiommata megera, Pityohyphantes alticeps, Gasteracantha cancriformis, Dryomyza anilis, Glossophaga soricina, Geomalacus maculosus, Microgastrinae gen. mgJanzen01 sp. Janzen01, Baetis adonis, Cricotopus tricinctus, Mastigias papua, Laricobius rubidus, Formica sp. BOLD: AAA1467, Drymeia sp. BOLD: AAC1021, Bradysia cellarum, Musca domestica, Balaustium murorum, Nautilus pompilius, Platynothrus peltifer, Simulium meridionale, Opisthorchis felineus, Mantis religiosa, Diplazon laetatorius, Dysstroma truncata, Monoporeia affinis, Astyanax mexicanus, Pampus argenteus, Anastrepha fraterculus, Suillia laevis, Megaselia sp. BOLD: AAG3274, Channa gachua, Leucotaraxis argenticollis, Macridiscus semicancellata, Phora sp. BOLD-2016, Thremma gallicum, Portunus pelagicus, Dendropoma gregarium, Dermacentor variabilis, Bicellaria bisetosa, Dolichopus sp. BOLD-2016, Chthamalus dentatus, Conioscinella triorbiculata, Limnephilus submonilifer, Dendrolimus punctatus, Bradysia excelsa, Ammodytes japonicus, Solenopsis geminata, Ochlerotatus caspius, Meretrix petechialis, Rugathodes sexpunctatus, Pollenia vagabunda, Ephemerella sp. AMI 1, Chrysomya albiceps, Arbacia lixula, Spodoptera litura, Ciona intestinalis, Velarifictorus micado, Forcipata acclina, Pareledone turqueti, Spirometra erinaceieuropaei, Acrocnida brachiata, Solenopsis invicta, Helina sp. BOLD: AAC2498, Odontaster validus, Scoparia sp. BOLD: AAA1518, Cyclosa conica, Panulirus argus, Rhopalopterum soror, Coenosia mollicula, Anopheles maculatus, Aethina tumida, Scutiger boulengeri, Chironomus entis, Stigmella corylifoliella, Sorhoanus xanthoneurus, Brachynopus scutellaris, Phyllocnistis sp. BOLD: AAF6337, Chelonibia testudinaria, Pectinaria koreni, Grandidierella japonica, Autographa gamma, Notemigonus crysoleucas, Corynoneura arctica, Dolichopus hastatus, Diabrotica virgifera, Ophionereis schayeri, Hymetta balteata, Dirofilaria repens, Chelonus sp. BOLD-2016, Hyalella azteca, Rastrelliger kanagurta, Contacyphon padi, Fannia brooksi, Idia aemula, Orientus ishidae, Gynaephora sp., Anguilla bicolor, Anystis agilis, Leptodactylus macrosternum, Microcosmus squamiger, Formica lasioides, Liriomyza brassicae, Aporia crataegi, Pleuromamma xiphias, Myrmica kotokui, Muscina sp. 1 AKR, Aegla alacalufi, Claustropyga acanthostyla, Aurelia aurita, Haliotis diversicolor, Hybomitra lasiophthalma, Schoenomyza sp. BOLD: AAG4622, Parazoanthus axinellae, Tipula paludosa, Melampus bidentatus, Copidosoma floridanum, Corbicula japonica, Tricimba melancholica, Scepticus uniformis, Siphonaria japonica, Crassostrea angulata, Jehlius cirratus, Nucella emarginata, Aphis aurantii, Rangifer tarandus, Megaselia sp. BOLD: AAG3235, Leptothorax sp. BOLD-2016, Aphidius sp., Orientallactaga sibirica, Austruca annulipes, Coenonympha tullia, Anabrus simplex, Rattus rattus, Aspidiotus rigidus, Sclomina erinacea, Eris militaris, Idotea ochotensis, Abyssorchomene distinctus, Crassicutis cichlasomae, Dichopygina aculeata, Melitaea didyma, Leucotaraxis piniperda, Dictyna arundinacea, Venusia cambrica, Gigantidas childressi, Micropsectra sp. BOLD: AAF7088, Paracladopelma winnelli, Oreina speciosa, Microtendipes pedellus, Gammarus balcanicus, Anagrus sp. BOLD-2016, Neotrygon kuhlii, Aleiodes sp. BOLD-2016, Xestia c-nigrum, Diolcogaster sp. BOLD-2016, Echinococcus multilocularis, Sylvicola cinctus, Neritina canalis, Boettcheria cimbicis, Lispocephala alma, Tetragnatha extensa, Baetis alpinus, Chaceon imperialis, Brevoortia tyrannus, Papilio zelicaon, Pardosa mackenziana, Psychoda alternata, Cephaloleia belti, Coralliophila violacea, Leptothrips sp. BOLD-2016, Chironomus sp. BOLD: AAB4581, Melaphis rhois, Plebejus argus, Bryotropha gemella, Chimarra aterrima, Eodiaptomus wolterecki, Terebralia palustris, Trichonephila clavipes, Plectostoma concinnum, Patella candei, Brevipalpus yothersi, Ramburiella hispanica, Orthocladius sp. BOLD: AAU6556, Helina fulvisquama, Emerita brasiliensis, Belvosia sp. Woodley04A, Bactrocera latifrons, Tanymastix stagnalis, Micrempis bomboxynon, Bactrocera carambolae, Draeculacephala sp. BOLD-2016, Pararge aegeria, Lordithon sp. CHUI, Polypedates leucomystax, Aurelia coerulea, Indoplanorbis exustus, Polypedilum sp. BOLD: AAM6206, Typhlocyba sp. BOLD-2016, Meimuna kuroiwae, Chironomus acidophilus, Lissonota coracina, Cymodusa distincta, Biston betularia, Riptortus pedestris, Eriphia ferox, Ovis aries, Culex modestus, Caprella mutica, Pimpla pedalis, Geukensia demissa, Gadus chalcogrammus, Chanodichthys recurviceps, Helina flavisquama, Conogethes punctiferalis, Frankliniella tritici, Lispocephala erythrocera, Pieris napi, Cottus schitsuumsh, Neocalanus cristatus, Euphilotes battoides, Phenacoccus solenopsis, Dendroctonus frontalis, Melanagromyza obtusa, Xestia smithii, Mycomya humida, Hybomitra pechumani, Laelaps giganteus, Hesperoperla pacifica, Euthynnus affinis, Ischnura senegalensis, Mycomya shermani, Coleotechnites starki, Notocelia culminana, Ixodes ovatus, Dryophytes suweonensis, Lasioglossum malachurum, Diaeretiella rapae, Halictus scabiosae, Chimarra obscura, Schizopyge nukiangensis, Bradysia browni, Sorex bedfordiae, Plebejus idas, Paratya australiensis, Herina nigribasis, Agriphila ruricolella, Brachycaudus helichrysi, Squalus acanthias, Philodromus cespitum, Botanophila sp. BOLD-2016, Bactrocera umbrosa, Eriocheir japonica, Ceratoscopelus warmingii, Pseudolaubuca sinensis, Branchipus cortesi, Pegohylemyia sp. BOLD-2016, Dicrostonyx groenlandicus, Trichogramma platneri, Osmia lignaria, Aphidius rhopalosiphi, Balclutha neglecta, Pelophylax saharicus, Palaemon antennarius, Hisparonia hystrix, Luehdorfia japonica, Habrobracon hebetor, Pardosa lapponica, Georissa similis, Drosophila simulans, Patelloa sp. xanthuraDHJ01, Gadus morhua, Carassius auratus, Apatania sp. BOLD: AAA8874, Paratanytarsus sp. BOLD: ACF2457, Scatopsciara sp. BOLD-2016, Poecilia mexicana, Orthocentrus sp. BOLD-2016, Cauloramphus magnus, Carcinus aestuarii, Melita nitida, Chrysoperla externa, Callosobruchus chinensis, Lonchoptera bifurcata, Helicoverpa zea, Dolichopus sp. SEB11, Chrysotus sp. BOLD: AAG9659, Chrysotus sp. BOLD: AAG6860, Gonatocerus sp. BOLD: AAG1488, Hesiolyra bergi, Ooctonus notatus, Sardinella longiceps, Scylla serrata, Radix auricularia, Melanoplus sp. BOLD-2016, Sicydium plumieri, Zaphne divisa, Halobates maculatus, Bathymodiolus septemdierum, Chrysomya rufifacies, Scatella stagnalis, Echinococcus canadensis, Owenia fusiformis, Forficula aff. auricularia A BOLD-2014, Rhyacophila fuscula, Notopterus notopterus, Cordilura fuscipes, Gammarus lacustris, Millepora alcicornis, Eulepetopsis vitrea, Coxiella glabra, Apamea devastator, Selar crumenophthalmus, Scarus forsteni, Tetramorium sp. U2 BK-2018, Mesodesma donacium, Tibellus maritimus, Aedes notoscriptus, Coxiella striatula, Rhipidomys macconnelli, Engraulis encrasicolus, Dikraneura sp. BOLD-2016, Parnassius apollo, Morpho epistrophus, Lispe tentaculata, Chthamalus moro, Phrynocephalus helioscopus, Dipteromimus tipuliformis, Pseudocalanus minutus, Pimelodus maculatus, Eptesicus fuscus, Ephoron shigae, Vanessa cardui, Leptopterna amoena, Pacifastacus leniusculus, Pomphorhynchus laevis, Oryzias curvinotus, Craterostigmus tasmanianus, Raja miraletus, Crassostrea virginica, Polypedilum sp. BOLD-2016, Hybomitra sp. BOLD: ACM8443, Bombina orientalis, Equus caballus, Quasipaa boulengeri, Rhithropanopeus harrisii, Galeocerdo cuvier, Valenzuela sp. BOLD: AAH3228, Bradysia urticae, Pieris brassicae, Vatesus cf. clypeatus 2 CVB-2015, Pardosa hyperborea, Gammarus setosus, Haemadipsa trimaculosa, Choeras sp. BOLD-2016, Chaitophorus stevensis, Meloidogyne enterolobii, Lysandra coridon, Penaeus indicus, Pimpla aequalis, Haemaphysalis flava, Formica sp. BOLD: AAA4977, Abudefduf vaigiensis, Argyrodes minax, Myotis lucifugus, Locusta migratoria, Larinioides patagiatus, Pratylenchus scribneri, Corbicula fluminea, Synopeas sp. BOLD-2016, Pachygrapsus crassipes, Nymphalis urticae, Mastacembelus armatus, Tanytarsus sp. BOLD: AAG5467, Meteorus sp. BOLD-2016, Thymelicus lineola, Hyalopterus arundiniformis, Thricops innocuus, Thor amboinensis, Ostrinia nubilalis, Systomus sarana, Balaustium sp. BOLD: ACW0874, Hydriomena furcata, Rhamphomyia pendens, Hydrobioides nassa, Glossogobius giuris, Limnius perrisi, Rhyacophila minora, Valenzuela burmeisteri, Squalius cephalus, Mythimna separata, Muscina levida, Sympycnus lineatus, Panulirus marginatus, Enterobius vermicularis, Mesocriconema xenoplax, Macrosteles tesselatus, Glabraster antarctica, Drupella cornus, Mythimna unipuncta, Idia rotundalis, Keratella cochlearis, Dendropoma platypus, Incertella incerta, Diplectrona modesta, Cylindrus obtusus, Smittia sp. 14ES, Chthamalus montagui, Modiolus modiolus, Brachidontes sp. lineage B CDL-2020, Drosophila antonietae, Calineuria californica, Parametriocnemus sp. BOLD: ACC5534, Parectecephala eucera, Heleobia atacamensis, Bolitophagus reticulatus, Nodularia douglasiae, Beringraja pulchra, Chthamalus proteus, Microplana terrestris, Coenonympha pamphilus, Procladius sp. BOLD: AAG5513, Hemigrapsus sanguineus, Trhypochthonius tectorum, Tanytarsus guerlus, Cricotopus obnixus, Dicosmoecus gilvipes, Conopomorpha cramerella, Pterois volitans, Simulium tani, Anopheles hinesorum, Siphonaria serrata, Patella caerulea, Phycomenes zostericola, Xiphias gladius, Bos taurus, Unio crassus, Helina troene, Strophosoma melanogrammum, Lepidocyrtus lanuginosus, Thomomydoecus minor, Tripneustes gratilla, Saccostrea echinata, Enoclerus nigripes, Gasterosteus aculeatus, Telegonus synecdoche, Agama agama, Vaejovis carolinianus, Synopeas sp. BOLD: AAU9048, Sparus aurata, Anopheles minimus, Tanytarsus bathophilus, Coryphopterus hyalinus, Littoraria sinensis, Ophiothrix suensoni, Emblyna peragrata, Megalaspis cordyla, Megaselia subpalpalis, Stramonita haemastoma, Succinea caduca, Halocaridina rubra, Tetragnatha shoshone, Elliptio hopetonensis, Spilogona sp. BOLD-2016, Branchinecta lynchi, Brachycaudus divaricatae, Mytella charruana, Helina subvittata, Boreogadus saida, Popenaias popeii, Bryotropha plantariella, Allodia sp. BOLD-2016, Gekko gecko, Cricotopus perniger, Parachironomus environmental sample, Telenomus sp. BOLD: ACM1917, Anopheles nuneztovari, Vespula germanica, Dendropsophus aff. minutus, Apodemus draco, Ligula intestinalis, Dolichogenidea sp. Janzen57, Hemerodromia conspecta, Anopheles gambiae, Biomphalaria tenagophila, Carabus lusitanicus, Argyresthia pygmaeella, Paratendipes sp. BOLD: AAG5473, Dolophilodes distinctus, Daktulosphaira vitifoliae, Teliapsocus conterminus, Isurus oxyrinchus, Thunnus thynnus, Megaselia sp. BOLD: ABU5528, Erythridula wysongi, Amphibalanus amphitrite, Chrysoteuchia topiarius, Chironomus melanescens, Pardosa fuscula, Carabus blaptoides, Oncorhynchus tshawytscha, Copelatus sp. BI-2011, Zonocyba pomaria, Telemiades fides, Anopheles sundaicus, Salmo trutta, Pegoplata nigroscutellata, Contacyphon laevipennis, Pustulosa pustulosa, Coenosia pilosissima, Ectropis crepuscularia, Ptecticus trivittatus, Tridacna noae, Trochosa terricola, Nipponaclerda biwakoensis, Aphidius colemani, Planococcus citri, Lineus sanguineus, Chaitophorus populeti, Gorgythion begga, Schistocerca americana, Brachidontes pharaonis, Acrosternum hilare, Paralicella tenuipes, Tridacna squamosa, Camponotus sp. BOLD-2016, Aristeus antennatus, Myrmica fracticornis, Trissolcus euschisti, Hyporthodus octofasciatus, Leptidea juvernica, Thrips flavus, Scylla olivacea, Periplaneta fuliginosa, Capitophorus elaeagni, Themisto abyssorum, Pteronarcella badia, Estrandia grandaeva, Bombyx mandarina, Culiseta annulata, Adelphocoris lineolatus, Ceratopsyche morosa, Scolonida gracilis, Catocala dissimilis, Tenebrio molitor, Monodonta perplexa, Tetragnatha straminea, Lasiurus semotus, Diplolepis rosaefolii, Stenopelmatus sp. ‘mahogani’, Thaisella chocolata, Simulium sp. BOLD: AAA8954, Euglossa iopoecila, Philarctus bergrothi, Talides sp. Burns04, Cheiracanthium mildei, Ixodes holocyclus, Oncorhynchus keta, Oxystele tigrina, Tricholochmaea sp. BOLD: AAG4420, Thaumatomyia trifasciata, Biston falcata, Lepyronia quadrangularis, Graphopsocus cruciatus, Forcipata loca, Coregonus clupeaformis, Decapterus macrosoma, Lasiorhynchus barbicornis, Neriene radiata, Madaglymbus sp. BI-2011, Blepharoneura sp. 4 MC-2007, Dendrobaena octaedra, Chironomus sp. TE13, Simulium arcticum, Micropsectra sp. BOLD-2016, Ammodytes heian, Rhyacophila tristis, Aromia bungii, Boloria eunomia, Protodeltote albidula, Pegomya geniculata, Leptocera neofinalis, Orthocladius oblidens, Megacopta cribraria, Hippocampus mohnikei, Pirenella cingulata, Pangrapta decoralis, Erebia epiphron, Cnemidocarpa verrucosa, Delia antiqua, Bradysia nitidicollis, Pseudorasbora parva, Baetis phoebus, Spilogona sp. BOLD: AAM9109, Oligonychus perseae, Nacella magellanica, Euura fallax, Lepidostoma togatum, Austropotamobius torrentium, Anageshna primordialis, Aphidius transcaspicus, Gesneria centuriella, Cirrhinus mrigala, Littorina brevicula, Carnus hemapterus, Perizoma grandis, Orchestia gammarellus, Platypalpus nr. hyaenoides BOLD-2016, Bombus ignitus, Anopheles aconitus, Neodryinus typhlocybae, Pseudokuhnia minor, Pheidole sp., Evechinus chloroticus, Tenuiphantes zelatus, Excirolana armata, Myrmica alaskensis, Clepsis virescana, Mydaea affinis, Chrysops sp. BOLD-2016, Philoscia muscorum, Trioza erytreae, Amrasca biguttula, Diplodus vulgaris, Pseudagrion microcephalum, Brachyhypopomus occidentalis, Acanthoscelides obtectus, Limnophyes pumilio, Ichneumon sp. BOLD-2016, Oithona similis, Clubiona abboti, Poeciliopsis infans, Cratomelus armatus, Pangasianodon hypophthalmus, Scottnema lindsayae, Erythroneura elegans, Calotes mystaceus, Gonioctena olivacea, Lasioglossum pauxillum, Limnophyes asquamatus, Forcipomyia bipunctata, Seriola lalandi, Monocelis lineata, Aglaoctenus lagotis, Alphomelon sp. xestopygaDHJ05, Neolissochilus hexagonolepis, Anopheles albitarsis, Auxis rochei, Macrocentrus sp. BOLD-2016, Paratanytarsus austriacus, Conus ventricosus, Antarctonemertes valida, Oncopsis sp. BOLD: ACI7197, Plectrocnemia cinerea, Antarctonemertes riesgoae, Ips typographus, Atya scabra, Lepthyphantes alpinus, Eurois astricta, Geisha distinctissima, Caridina typus, Tubifex tubifex, Patella rustica, Zaphne sp. BOLD: AAG2434, Idia americalis, Hypenodes palustris, Diadema africanum, Donax hanleyanus, Duttaphrynus melanostictus, Lucilia cuprina, Fabriciana adippe, Lycaena phlaeas, Bryotropha sp. BOLD-2016, Chaoborus trivitattus, Pemphigus sp. BOLD-2016, Erigone psychrophila, Brachionus sp. Manjavacas, Pyura praeputialis, Dyspanopeus sayi, Myodes rufocanus, Celleporella hyalina, Telegonus Augeas, Anadara tuberculosa, Apallates coxendix, Emblyna sublata, Cosmopterix sp. BOLD: ABZ0184, Petasiger sp. 5 ML, Speranza loricaria, Mullus barbatus, Panulirus interruptus, Suillia quinquepunctata, Erebia euryale, Lasiurus borealis, Ceratopsyche oslari, Colias croceus, Cinara pinea, Agrotis ruta, Xanthonia decemnotata, Anisakis physeteris, Balclutha punctata, Lambdina sp. BOLD: AAA5482, Sardinella lemuru, Calomera littoralis, Rhagada perprima, Macrosiphum euphorbiae, Heliconius melpomene, Rhynchobatus australiae, Synchaeta pectinata, Octopus insularis, Bracon sp., Sepsis punctum, Dictyna volucripes, Branchipolynoe symmytilida, Agrius convolvuli, Zodarion styliferum, Lutzomyia longipalpis, Artibeus glaucus, Pseudotelphusa belangerella, Sarraceniomyia sarraceniae, Thrips vulgatissimus, Macrobrachium japonicum, Astyanax lacustris, Polyarthra dolichoptera, Junco hyemalis, Schausiella santarosensis, Synisoma nadejda, Trachurus picturatus, Cryptasterina hystera, Dichomeris punctipennella, Valisia javana, Aequiyoldia eightsii, Palaemon elegans, Erigone aletris, Diplocentria bidentata, Derotanypus alaskensis, Bombus pauloensis, Flaccisagitta enflata, Malacosoma disstria, Clibanarius virescens, Eupeodes sp. BOLD-2016, Gammarus sp. t TM-2021, Culiseta inornata, Euschistus tristigmus, Pityohyphantes subarcticus, Ligyrocoris diffusus, Halobates hayanus, Coralliophila radula, Pterois miles, Halysidota tessellaris, Ephedrus lacertosus, Elachista illectella, Ototyphlonemertes lactea, Neopomacentrus cyanomos, Dreissena presbensis, Ceratopogon sp. BOLD-2016, Acrocercops astericola, Sus scrofa, Oryzomys couesi, Cerastoderma glaucum, Bradysia flavipila, Tetrancistrum nebulosi, Lottia digitalis, Forcipomyia titillans, Alburnus alburnus, Decma fissa, Myrmica ruginodis, Proechimys cuvieri, Nucella lapillus, Carollia brevicauda, Parvulastra exigua, Petromyzon marinus, Scathophaga sp. BOLD-2016, Stenotus binotatus, Eacles sp. Janzen02, Araniella displicata, Perna viridis, Agapetus ochripes, Parakiefferiella sp. BOLD: AAI2683, Dolichovespula arenaria, Acrocercops transecta, Araneus venatrix, Polypedilum sp. BOLD: ACK7314, Calcinus haigae, Aratus pisonii, Nelima paessleri, Nymphon australe, Astyanax bimaculatus, Chionoecetes japonicus, Raja clavata, Simulium sp. BOLD: AAA1697, Ophiomitrella clavigera, Sinonovacula constricta, Scylla tranquebarica, Paracaprella pusilla, Lasioglossum leucozonium, Bradysia atroparva, Celastrina argiolus, Phyllocnistis labyrinthella, Calanoides natalis, Eukiefferiella sp. BOLD-2016, Melanostoma mellinum, Thrips sp. BOLD-2016, Protaetia brevitarsis, Limnophila sp. BOLD: ABX6753, Muscina pascuorum, Corynoptera globiformis, Zaprionus indianus, Acroclita subsequana, Unio mancus, Rhamdia quelen, Aricia artaxerxes, Hybomitra frontalis, Melipona quadrifasciata, Lachesilla pedicularia, Musca autumnalis, Sceptonia sp. BOLD-2016, Euura vaga, Oncorhynchus kisutch, Tetramorium caespitum, Stictococcus vayssierei, Anopheles messeae, Tetragnatha viridis, Etheostoma nigrum, Pimephales promelas, Limnephilus externus, Trichatelura manni, Dialipsis dissimilis, Coquillettidia perturbans, Anopheles marajoara, Creteuchiloglanis macropterus, Megaselia eccoptomera, Baetis bicaudatus, Corynoptera subcavipes, Homaemus aeneifrons, Patella ulyssiponensis, Pityohyphantes costatus, Pseudoplatystoma corruscans, Holothuria inornata, Phyllocnistis citrella, Mediodactylus kotschyi, Leptus sp. BOLD: AAM7977, Hyalomma truncatum, Lespesia aletiae, Noctua pronuba, Labeo catla, Symbion americanus, Elachiptera decipiens, Agrochola bicolorago, Diapterobates humeralis, Anyphaena pectorosa, Blepharoneura sp. 30 MC-2007, Ridgeia piscesae, Hymenosoma longicrure, Costaconvexa centrostrigaria, Agrotis ipsilon, Reticulitermes grassei, Cloeon dipterum, Graphiphora augur, Coptotermes formosanus, Heteroxynema cucullatum, Cactoblastis cactorum, Dinotrema sp. jft01, Platorchestia japonica, Lygus borealis, Oreochromis mossambicus, Chorthippus binotatus, Gymnometriocnemus sp. BOLD: AAM6261, Artemia urmiana, Octopus berrima, Microplitis sp. BOLD-2016, Prays oleae, Lampides boeticus, Bourletiella sp. BOLD: ACO4150, Aphidius sp. BOLD-2016, Phrynocephalus guttatus, Pityogenes chalcographus, Wiebesia pumilae, Phoenicurus phoenicurus, Psyche casta, Atlantorchestoidea brasiliensis, Birgus latro, Peyerimhoffia vagabunda, Cheilodipterus quinquelineatus, Hoplias malabaricus, Cervus nippon, Eupithecia cretaceata, Lygus hesperus, Mysis salemaai, Corynoneura sp. BOLD-2016, Ligia exotica, Simulium nodosum, Thymelicus sylvestris, Polyommatus celina, Psychoda phalaenoides, Xysticus elegans, Myrmica sp. GJ-2009, Thaumatomyia sp. BOLD-2016, Neocaridina denticulata, Solenopsis sp. 15 of SKY, Papilio zelicaon x Papilio machaon, Inurois punctigera, Ceratoppia sp. BOLD: ACT8816, Philodromus peninsulanus, Pteronarcys californica, Tor tor, Erythroneura bakeri, Hybomitra epistates, Anguilla marmorata, Chrysodeixis includens, Metridia lucens, Gluphisia septentrionis, Enypia venata, Rana dybowskii, Pontoscolex corethrurus, Panonychus ulmi, Lamellibrachia sp. 2 BB-2017, Sipunculus nudus, Sinotaia aeruginosa, Thaumetopoea pityocampa, Zaglyptus varipes, Crambus albellus, Callosobruchus maculatus, Lauxania shewelli, Prochilodus hartii/argenteus sp., Meretrix meretrix, Aricia cramera, Blepharoneura sp. 3 MC-2007, Tetrastichus atratulus, Thricops villicrura, Malacosoma californicum, Thrips parvispinus, Plectus murrayi, Frankliniella intonsa, Pelagia noctiluca, Aedes koreicus, Lethocerus indicus, Euwallacea fornicatus, Bombyx mori, Cotesia sp. BOLD-2016, Liriomyza huidobrensis, Dryomyza setosa, Bokermannohyla saxicola, Schizaphis graminum, Orthocladius olivaceus, Rhipicephalus turanicus, Cricotopus vierriensis, Grapholita molesta, Ladislavella elodes, Acontias meleagris, Liriomyza sp. BOLD-2016, Xestia speciosa, Boettcheria bisetosa, Delia florilega, Cicadula intermedia, Dolichopus brevipennis, Rhinolophus affinis, Cratyna uliginosoides, Geina tenuidactyla, Pleurobema clava, Panorpodes paradoxus, Hiatella sp. L HML-2015, Apocheima cinerarius, Aritranis director, Glossosoma nigrior, Kleidocerys resedae, Antilope cervicapra, Xenopus laevis, Melanostoma sp. BOLD: AAB2866, Alismobates inexpectatus, Euhybus triplex, Trochulus hispidus, Zenopontonia soror, Nipponacmea fuscoviridis, Lucilia illustris, Haploniscus bicuspis, Cheumatopsyche sp. AMI 1, Cephaloflexa bergi, Dibothriocephalus latus, Mesembrina latreillii, Pardosa xerampelina, Lacinipolia renigera, Metzneria lappella, Pachycondyla sp. MAS017, Sarcophaga peregrina, Rhabdomys pumilio, Trixagus sp. BOLD-2016, Agraylea multipunctata, Pseudothyatira cymatophoroides, Cochliomyia hominivorax, Chaetodipus spinatus, Hydrophoria sp. BOLD-2016, Gymnodia humilis, Opsiphanes cassina, Polyphemus pediculus, Araneus nordmanni, Amblygaster sirm, Dicerapanorpa magna, Chrysoritis chrysaor, Nippostrongylus brasiliensis, Ochlerotatus excrucians, Goera calcarata, Macrotermes subhyalinus, Choristoneura rosaceana, Psathyromyia shannoni, Sphaerophoria sp. BOLD-2016, Nerita yoldii, Deroceras leave, Araneus marmoreus, Brachionus quadridentatus, Glaucopsyche alexis, Feltia jaculifera, Heterocarpus reedi, Actia diffidens, Eukiefferiella sp. BOLD: ABZ4061, Nemipterus furcosus, Eupithecia subfuscata, Heteromys desmarestianus, Calliphora vomitoria, Sarcophaga dux, Hylemyza partita, Glossina pallidipes, Triplophysa robusta, Hepialus humuli, Anthonomus grandis, Automeris sp. Janzen01, Adelges nordmannianae, Hippodamia convergens, Neritina granosa, Mycetophila alea, Culex annulirostris, Bradysia hilaris, Cercyonis lycaon, Isotoma riparia, Coleotechnites piceaella, Orthocladius dorenus, Listronotus oregonensis, Pentalonia nigronervosa, Raphia frater, Scoliodon laticaudus, Epinephelus coioides, Philodina megalotrocha, Polypedilum nubifer, Acutisoma longipes, Acyrthosiphon pisum, Thricops diaphanus, Anablepsoides hartii, Lutjanus kasmira, Spilogona imitatrix, Dalopius sp. BOLD: AAH2388, Piratula insularis, Ctenocephalides canis, Bathymodiolus heckerae, Anopheles barbirostris, Heliconius cydno, Scutellastra granularis, Diamesa arctica, Coryphopterus glaucofraenum, Katharina tunicata, Steganacarus magnus, Brevicornu sp. BOLD-2016, Pollicipes elegans, Lordithon sp. BOLD: AAG4307, Caridina pseudodenticulata, Parastacus nicoleti, Oophaga sylvatica, Larinioides cornutus, Scleropages formosus, Thricops sp. BOLD: ABY7837, Chironomus circumdatus, Chrysomela lapponica, Xestospongia muta, Dendroctonus valens, Elaphe climacophora, Anopheles peditaeniatus, Rhagada convicta, Tomicus destruens, Dyscophellus sp. phraxanorDHJ02, Fannia sp. BOLD-2016, Harpadon nehereus, Trioxys pallidus, Capitulum mitella, Penaeus vannamei, Junonia coenia, Channa punctata, Necator americanus, Aedes flavopictus, Agabus brunneus, Pyrgus alveus, Pteroptyx tener, Micropsectra recurvata, Parantica sita, Scarus oviceps, Gynaephora qinghaiensis, Lonchaea sp. BOLD-2016, Lampsilis teres, Bradysia sp. BOLD: ACC2902, Kryptolebias hermaphroditus, Squalidus argentatus, Melanostoma sp. BOLD-2016, Osmoderma barnabita, Mugil curema, Aeolothrips sp. BOLD-2016, Clarkcoma canaliculata, Nadata gibbosa, Clausicella setigera, Scirtes tibialis, Neocalanus flemingeri, Glyptapanteles sp. BOLD-2016, Cricotopus tremulus, Jenynsia lineata, Proterebia afra, Pimpla sp. BOLD: AAD5193, Opisthopatus cinctipes, Limnonectes modestus, Lyonetia pulverulentella, Clarias gariepinus, Metacnephia borealis, Thaumatotibia batrachopa, Bentholebouria georgenascimentoi, Decapterus maruadsi, Ourapteryx ebuleata, Didemnum vexillum, Drosophila sp. BOLD: ACF0720, Canis lupus, Wanella milleporae, Ceraticelus atriceps, Enteromius cf. mimus HM-2020, Tvetenia paucunca, Olethreutes glaciana, Formica aserva, Fusconaia flava, Mompha sp. JFL01, Eukiefferiella sp. BOLD: ABA1245, Sinanodonta woodiana, Apanteles sp. Rodriguez26, Bos grunniens, Hollandichthys multifasciatus, Cerodontha dorsalis, Halobates micans, Chilo suppressalis, Pteromicra glabricula, Juga plicifera, Strongyloides fuelleborni,, and/or Longitarsus ordinatus.

In some aspects, a metabarcoding database of the disclosure further comprises additional unique COI nucleic acid sequences associated with eukaryotes, particularly arthropods and certain genuses/species that can be present in crops, fields, and farms. In some aspects, a database of the disclosure was annotated with 100 to 199 unique COI sequences from the following arthropod genus: Chaetodon melapterus, Diploschizia impigritella, Spilogona sp. BOLD: AAG3154, Kerivoula cf. hardwickii CMF-2010, Eurois occulta, Melanargia galathea, Siphonaria pectinata, Phytocoris neglectus, Leucetta chagosensis, Naonella forsythi, Trypoxylon frigidum, Neokolla hieroglyphica, Lepeophtheirus salmonis, Culicoides brevitarsis, Manis javanica, Cheumatopsyche oxa, Parechinus angulosus, Lens contradens, Mobula japanica, Monomorium cyaneum, Paramyia nitens, Cheumatopsyche harwoodi, Opecarcinus hypostegus, Helina annosa, Astacus leptodactylus, Anopina sp. BOLD: AAE5367, Camallanus cotti, Euschistus heros, Paracalliope fluviatilis, Anoplolepis gracilipes, Euphilotes pallescens, Platyrrhinus helleri, Busseola fusca, Hypena baltimoralis, Lepidocyrtus sp. BOLD: AAI3390, Upogebia africana, Drosophila meridionalis, Sphoeroides testudineus, Smittia sp. ES11, Colymbetes dolabratus, Allacma fusca, Betta splendens, Boeckella poppei, Dusona sp. BOLD: AAC9245, Myrmecina americana, Coleophora duplicis group sp. BOLD-2016, Pristiphora cincta, Clibanarius tricolor, Lymnaea stagnalis, Platynota idaeusalis, Emblyna annulipes, Nasonia vitripennis, Hyporhamphus australis, Enteroctopus dofleini, Asteromyia carbonifera, Lophiotoma acuta, Tegillarca granosa, Platambus maculatus, Ephestia kuehniella, Megaloprepus caerulatus, Niphargus stygius, Monochamus galloprovincialis, Crassostrea ariakensis, Gammarus pulex, Aporrectodea rosea, Clio pyramidata, Lygocoris pabulinus, Ephemerella dorothea, Kapentagyrus tanganicanus, Callizzia amorata, Carpetania elisae, Phygadeuon trichops, Bucculatrix sp. BOLD-2016, Heteranassa mima, Eilema ussuricum, Merops apiaster, Eulithis explanata, Cyamus ovalis, Ophiactis abyssicola, Octolasmis angulata, Sebastes sp., Scomber japonicus, Empoasca vitis, Pheidole megacephala, Altica tombacina, Neoponera villosa, Barilius bendelisis, Delia linearis, Cratyna cryptospina, Anopheles coustani, Grimothea gregaria, Euproctus montanus, Sturnira tildae, Hsunycteris thomasi, Rhizoprionodon acutus, Toxocara canis, Myzus cerasi, Neophyllomyza sp. BOLD-2016, Bezzia sp. BOLD-2016, Australatya striolata, Epaphius chalybeus, Limnadopsis parvispinus, Morethia ruficauda, Anaplectoides prasina, Diplostomum sp. 1 SAL-2008, Cricotopus sp. BOLD: AAU6761, Leuctra sp. BOLD: AAC1685, Habrosyne scripta, Rhantus suturalis, Typhlocyba rosae, Lycaena tityrus, Lampsilis hydiana, Glycaspis brimblecombei, Marthasterias glacialis, Pigritia sp. BOLD: AAC6102, Blattella germanica, Neophilaenus lineatus, Liponeura cinerascens, Orthosia hibisci, Digrammia curvata, Tevnia jerichonana, Melanoides tuberculata, Ephestiodes gilvescentella, Niphargus schellenbergi, Thunnus alalunga, Squalus blainville, Creagrura alejandromasisi, Sardina pilchardus, Amathia verticillata, Simulium aureohirtum, Automeris sp. postalbidaDHJ07, Lumbricus castaneus, Thysanoessa inermis, Callophrys rubi, Hyalomma excavatum, Agrothereutes abbreviatus, Pomacea insularum, Ceraticelus fissiceps, Saccocirrus uchidai, Trichostrongylus axei, Procambarus paeninsulanus, Pnigalio mediterraneus, Loxostege sticticalis, Anacampsis conclusella, Siphona intrudens, Orfelia nemoralis, Anopheles baimaii, Chionodes mediofuscella, Sicyopus zosterophorus, Alaptus sp. BOLD-2016, Eilema bicolor, Chiastopsylla rossi, Eriocheir sinensis, Agyrtodes labralis, Mactra quadrangularis, Drosophila subquinaria, Creontiades dilutus, Psalidodon fasciatus, Echinometra mathaei, Xeropicta derbentina, Ducetia japonica, Metaphire sieboldi, Helina cinerella, Trochulus villosus, Ceratina sp. BOLD: AAA2368, Anoplopoma fimbria, Lepetodrilus ovalis, Timema cristinae, Amynthas morrisi, Tanytarsus oscillans, Sesamia inferens, Agriades optilete, Empoasca decipiens, Porites lutea, Adalia bipunctata, Xyleborinus saxesenii, Eratoneura aculeata, Ototyphlonemertes martynovi, Patelloida saccharina, Dichelotarsus extremus, Anguilla anguilla, Cladotanytarsus sp. BOLD-2016, Cuculus canorus, Pyrgus communis, Apamea cogitata, Hyalomma asiaticum, Danio rerio, Pigritia fidella, Acanthochitona garnoti, Glyptapanteles sp. Whitfield131, Reticulitermes speratus, Macrosiphum pseudocoryli, Lecanocerus compressiceps, Pratylenchus penetrans, Canalidion montanum, Caripeta divisata, Lacinipolia olivacea, Leucania insueta, Isotoma viridis, Ravinia querula, Carassius gibelio, Nucella lamellosa, Lepidostoma pictile, Coenagrion mercuriale, Aporrectodea longa, Schistosoma haematobium, Angiostrongylus cantonensis, Simulium verecundum complex sp. BOLD-2016, Tuberolachnus salignus, Chanos chanos, Coryphella verrucosa, Bactrocera tryoni, Ceratopsyche alternans, Hypena scabra, Eratoneura flexibilis, Sylvicola sp. BOLD: AAG1996, Melanterius maculatus, Bombus ephippiatus, Sternochetus mangiferae, Barbodes binotatus, Dendropoma rhyssoconchum, Telegonus inflatio, Amphinemura linda, Cosmocomoidea morrilli, Kikihia sp. ‘flemingi’, Lucidota atra, Urbanus sp. belliDHJ01, Oxyloma sp. HWH-2012, Thaumatotibia leucotreta, Neoscona theisi, Phaonia luteva, Peromyscus yucatanicus, Cimex hemipterus, Anopheles vagus, Anopheles subpictus, Diarsia esurialis, Opisthorchis viverrini, Camptochaeta flagellifera, Scarus schlegeli, Xanthorhoe ferrugata, Punctoribates punctum, Drosophila maculifrons, Phragmatopoma caudata, Pomacentrus coelestis, Ribautiana sp. BOLD-2016, Coleotechnites sp. BOLD: AAA5953, Boloria selene, Channa marulius, Dermacentor marginatus, Procladius sp. BOLD-2016, Cladotanytarsus amandus, Artibeus cinereus, Mastomys sp. IN-2012, Praomys misonnei, Cerapanorpa brevicornis, Renia adspergillus, Anacrusis sp. nephrodesDHJ05, Forcipomyia sp. BOLD: AAG6501, Polistes dominula, Phlebotomus perniciosus, Pseudanostirus triundulatus, Megaphthalma pallida, Meloidogyne graminicola, Drosophila sturtevanti, Triatoma infestans, Pherbellia sp. BOLD: AAG6911, Brachidontes exustus, Ototylomys phyllotis, Thrips sp. BOLD: AAG0736, Phyllodesma americana, Discocyrtus dilatatus, Uroleucon sp. BOLD-2016, Polydora websteri, Sterechinus neumayeri, Unio pictorum, Gammarus tigrinus, Hydra oligactis, Limonia nubeculosa, Sancus acoreensis, Fenusella nana, Eacles imperialis, Wangiannachiltonia guzikae, Pontogammarus maeoticus, Eurema hecabe, Enargia decolor, Chironomus plumosus, Ceratodalia gueneata, Onchidium reevesii, Okeniella sp. BOLD: AAH4222, Praon sp. BOLD-2016, Drosophila mojavensis, Chromis margaritifer, Lachesilla sp. BOLD-2016, Cynomya sp. BOLD: AAB0868, Pantelozetes cavaticus, Halichoeres hortulanus, Atteva aurea, Tylos granulatus, Anopheles koliensis, Mercenaria mercenaria, Tenthredo stricklandi, Placobdella rugosa, Mycetophila sp. BOLD: AAG4978, Saxidomus purpurata, Anisocentropus kawamurai, Carystoides sp. escalanteiDHJ02, Pseudomyrmex aff. flavicornis-a MAS-2013, Clastoptera obtusa, Chaetodon pictus, Proteus anguinus, Gouania hofrichteri, Acleris variana, Omiodes cuniculalis, Neurigona tenuis, Coleophora sp. BOLD-2016, Telenomus sp. BOLD: AAG7892, Parapharyngodon cubensis, Dicentria sp. rusticaDHJ05, Scathophaga sp. BOLD: AAH4215, Argosarchus horridus, Perichares adela, Lucilia eximia, Pemphigus betae, Epistranus lawsoni, Drusus discolor, Osteolaemus tetraspis, Sciara humeralis, Melaphis asafitchi, Spelobia ochripes, Hyles gallii, Bathynomus giganteus, Sesamia nonagrioides, Solenosteira macrospira, Anochetus_grandidier, Deronectes moestus, Pratylenchus vulnus, Ecliptopera silaceata, Sarcophaga albiceps, Erebia pronoe, Hippasteria phrygiana, Diplosoma listerianum, Cellana exarata, Mytilisepta virgata, Pirata praedo, Bicyclus anisops, Hymeniacidon perlevis, Maculabatis gerrardi, Pleurodema diplolister, Sylvicola alternatus, Zeuxapta seriolae, Rhagada sp. ‘Pilbara Banded’, Cleantiella isopus, Triops cancriformis, Amazona aestiva, Agallia quadripunctata, Hemideina maori, Neocaridina davidi, Bradysia tilicola, Cerithidea decollata, Bradina atopalis, Orgyia pseudotsugata, Spargania luctuata, Alopias pelagicus, Leguminivora glycinivorella, Lissonota recurvariae, Dolichopus sp. BOLD: AAG9634, Bracon sp. BOLD-2016, Limonius aeger, Harmothoe aff. imbricata CMC01, Arion subfuscus, Parisotoma aff. notabilis LI, Apantesis nevadensis, Delia bucculenta, Pleurobema riddellii, Anisotremus interruptus, Anabolia bimaculata, Rhaphium sp. BOLD-2016, Opsolasia orichalcea, Cletus punctiger, Sorex fumeus, Helina reversio, Trimerotropis pallidipennis, Cryptochironomus sorex, Tanytarsus sp. BOLD: AAE1904, Neverita didyma, Diplostomum spathaceum, Diadegma insulare, Lophostoma silvicolum, Palaemonella rotumana, Prosciara prosciaroides, Diabrotica undecimpunctata, Tabanus bromius, Dermogenys sp. MDB-2013, Trachurus trachurus, Etheostoma olmstedi, Gaurax pallidipes, Smidtia fumiferanae, Epirrhoe alternata, Pellioditis marina, Dichomeris sp. BOLD: AAH4942, Bathythrix fragilis, Melitaea cinxia, Cinara mariana, Rhyacophila angelita, Lithodes sp., Ochthebius quadricollis, Diachasma alloeum, Psectrotanypus sp. ESO1, Stylocheiron elongatum, Clubiona obesa, Delia extensa, Argynnis aglaja, Dunama sp. mexicanaDHJ02, Cupido minimus, Protoboarmia porcelaria, Rotaria macrura, Parametriocnemus sp. BOLD-2016, Xiphinema index, Glossogobius aureus, Lobophyllia corymbosa, Micrurapteryx salicifoliella, Theridion frondeum, Periclistus sp. BOLD-2016, Harmothoe fuligineum, Nemipterus randalli, Pomacea maculata, Schilbe intermedius, Oreohelix subrudis, Puntius sophore, Theodoxus fluviatilis, Adelges piceae, Trichoferus campestris, Phyllidiella pustulosa, Anopheles squamosus, Athetis lepigone, Charybdis hellerii, Eupithecia graefii, Polypedilum sp. ‘nubifer lookalike’, Spialia sertorius, Ensis directus, Coenosia sp. BOLD: ABX5997, Meloe strigulosus, Ameiurus melas, Pardosa dorsalis, Stenocranus felti, Zelandoperla fenestrata, Parapediasia teterrellus, Fannia armata, Enallagma hageni, Calliptamus barbarus, Aristotelia rubidella, Leporinus piau, Hetaerina americana, Bombus pascuorum, Sympycnus sp. BOLD: AAU6576, Blacus stelfoxi, Onithochiton neglectus, Bombus melanopygus, Erpobdella obscura, Cruzia tentaculata, Acetes chinensis, Glyphidocera septentrionella, Calomicrus circumfusus, Macrobrachium amazonicum, Omphisa fuscidentalis, Culicoides oxystoma, Palythoa tuberculosa, Peridroma saucia, Meghimatium fruhstorferi, Kuhnia scombri, Diplodus capensis, Parornix sp. BOLD-2016, Psilotreta indecisa, Iteaphila macquarti, Megaselia sp. 24 LL-2023a, Tachina sp. BOLD-2016, Peronia verruculata, Dolichovespula maculata, Diadegma sp. BOLD-2016, Halobates calyptus, Limnephilus hageni, Helina laxifrons, Campoletis sp. BOLD-2016, Protoreaster nodosus, Pelophylax ridibundus, Nyssomyia umbratilis, Calcinus hazletti, Carcharodus alceae, Anthocharis cardamines, Psammotettix sp. BOLD: AAG8814, Pheidole sp. MAS009, Euplexia benesimilis, Agrotis venerabilis, Haplorchis taichui, Rhizoprionodon porosus, Leptus sp. BOLD: ACE3889, Selaroides leptolepis, Brachidontes rostratus, Lysiphlebus testaceipes, Tinca tinca, Apodemus chevrieri, Tanganicodus irsacae, Cricotopus sylvestris, Gammarus stankokaramani, Culter alburnus, Kikihia cutora, Scylla paramamosain, Saissetia oleae, Mycetophila sp. CJB5, Dallia pectoralis, Bulia deducta, Primnoa pacifica, Thricops sp. BOLD: AAB6566, Neottialges evansi, Leptodora kindtii, Macroplea mutica, Lutjanus russellii, Melanaphis sorghi, Anavitrinella pampinaria, Westralunio carteri, Tetranychus evansi, Dicrotendipes nervosus, Pontogammarus robustoides, Anadara broughtonii, Rasbora lateristriata, Folsomia quadrioculata, Trichodezia albovittata, Rheosmittia spinicornis, Cinygmula subaequalis, Erythroneura ontari, Philodina gregaria, Orthocladius roussellae, Coptodon zillii, Ascogaster sp. BOLD-2016, Anopheles maculipennis, Hypenodes sp. n. 4 JRD-2015, Diopatra cuprea, Hydrotaea militaris, Monomorium rothsteini, Artibeus jamaicensis, Liophloeus gibbus, Mesochorus sp. BOLD-2016, Ochlodes sylvanus, Omiodes sp. humeralisDHJ07, Simulium asakoae, Aspidodera raillieti, Metridia pacifica, Euceraphis sp. BOLD: AAH2870, Bombus bifarius, Grammotaulius interrogationis, Sarcophaga argyrostoma, Angiopolybia pallens, Laelaps muricola, Holothuria arguinensis, Balanus crenatus, Elliptio icterina, Hippocampus trimaculatus, Solenopsis mameti, Laeonereis culveri, Pollenia griseotomentosa, Camponotus nearcticus, Tetradonia laticeps, Sminthurinus elegans, Rhyacophila fasciata, Phidippus johnsoni, Taenia multiceps, Psephenus herricki, Calliphora livida, Melanogrammus aeglefinus, Fletcherimyia fletcheri, Belvosia sp. Woodley02, Clepsis persicana, Bulinus globosus, Fannia sociella, Bathygobius soporator, Phlebotomus papatasi, Megaselia shatesae, Crystallias matsushimae, Biomphalaria glabrata, Anisakis typica, Biomphalaria straminea, Xenograpsus testudinatus, Psectrocladius barbimanus, Carposina sasakii, Bolinopsis microptera, Platypalpus pulicarius, Alpheus lottini, Dipus sagitta, Ctenochaetus striatus, Scepticus tigrinus, Haloa japonica, Aeorestes cinereus, Suillia sp. JKS-346, Armadillo officinalis, Dugesia sicula, Epirrita autumnata, Ophiura sarsii, Sitobion sp. BOLD-2016, Globodera rostochiensis, Galba truncatula, Loxosceles caribbaea, Thyca crystallina, Nerodia erythrogaster, Katsuwonus pelamis, Culex territans, Ceratopsyche slossonae, Heliconius erato, Metacrangonyx longipes, Pagurus bernhardus, Octopus hubbsorum, Phelotrupes laevistriatus, Procambarus spiculifer, Aphelinus sp. BOLD-2016, Apolygus lucorum, Bathycoelia distincta, Juga nigrina, Meloidogyne chitwoodi, Mylothris jacksoni, Telegonus fruticibus, Amblyraja radiata, Ellychnia sp. BOLD: ACV4844, Diplodus sargus, Tegula funebralis, Episyrphus balteatus, Omocestus petraeus, Drepanosiphum platanoidis, Elachista sp. BOLD: AAI6200, Echinoderes sp. HY 2014c, Isoperla sp. AMI 1, Orthodes cynica, Hybomitra sp. BOLD: AAB1544, Gotocotyla sawara, Asota heliconia, Bleptina caradrinalis, Millepora dichotoma, Oryctes rhinoceros, Blastobasis glandulella, Dusona minor, Tylosurus crocodilus, Eulimnogammarus cyaneus, Entomobrya sp. BOLD: AAA7246, Hemerobius sp. BOLD-2016, Pirata piraticus, Ischnura elegans, Isotomurus plumosus L2, Leptopeza flavipes, Pachycoris torridus, Variabilichromis moorii, Trichodorus obtusus, Arbacia spatuligera, Rhagonycha mollis, Parapanteles tessares, Sargus decorus, Phrynus longipes, Spirobranchus triqueter, Culiseta longiareolata, Shinkailepas tollmanni, Nassaria sp. 1 MC-2010, Parakiefferiella scandica, Penaeus merguiensis, Brachyponera chinensis, Ephemerella invaria, Aspidiotus nerii, Aplocheilus panchax, Atractodes sp. BOLD-2016, Hyalomma rufipes, Xylophanes sp. Janzen03, Typhlocyba melite, Mythimna oxygala, Perumytilus purpuratus, Neobisium carcinoides, Haplothrips leucanthemi, Geomitopsis sp., Calathus ingratus, Cheumatopsyche analis, Polyplax praomydis, Schizopygopsis malacanthus, Orchesella cincta, Haliotis discus, Telenomus sp. BOLD: AAU9275, Junonia evarete, Gasterophilus pecorum, Pan troglodytes, Platynereis dumerilii, Lanius collurio, Pseudocalanus mimus, Stigmella myricafoliella, Cyanoliseus patagonus, Suillia barberi, Turdus viscivorus, Rejectaria sp. Janzen06, Shinkaia crosnieri, Ptyas dhumnades, Doris kerguelenensis, Diplostyla concolor, Hemimysis margalefi, Callitettix versicolor, Synema globosum, Leptodactylus bufonius, Gobiesox adustus, Teredo navalis, Aedes sp. BOLD: AAB1098, Lasiomma cuneicorne, Solenopsis saevissima, Clarias batrachus, Epinephelus areolatus, Rosema attenuata, Lepetodrilus concentricus, Misgurnus anguillicaudatus, Ochropleura implecta, Sinotaia cf. quadrata BBY-2019, Gonepteryx rhamni, Anopheles tessellatus, Cheilomenes sexmaculata, Pardosa groenlandica, Larinus aff. minutus/obtusus, Acleris curvalana, Pyrops candelaria, Lasiommata maera, Neolucanus swinhoei, Bryophaenocladius ictericus, Ruditapes decussatus, Dosidicus gigas, Palaemon varians, Agonopterix pulvipennella, Unio elongatulus, Pero morrisonaria, Mansonia titillans, Blepharoneura sp. 21 MC-2007, Clytus ruricola, Gorilla gorilla, Coenosia conforma, Baetis vernus, Pseudapanteles sp. Whitfield19, Amphinemura sp. AMI 1, Labrus bergylta, Agrypnia colorata, Ophiothrix angulata, Colias fieldii, Mocis latipes, Biston suppressaria, Ponera swezeyi, Trogloneta yunnanense, Spilogona firmidisetosa, Vignadula atrata, Piratula minuta, Coenosia sp. 1 AKR, Procladius sp. ES02, Hydrobius fuscipes, Promachocrinus sp. f ELM-2023, Belvosia sp. Woodley03C, Xysticus emertoni, Uroderma bilobatum, Pagurus hirsutiusculus, Heliothis virescens, Petrolisthes armatus, Rhectocraspeda periusalis, Ranoidea nannotis, Metalimnobia immatura, Thymelicus acteon, Alpheus japonicus, Arion fuscus, Dentex dentex, Eutrichota lipsia, Chrysops sp. BOLD: AAD4375, Saccopteryx bilineata, Crangon crangon, Bithoracochaeta calopus, Xeruca formosensis, Leiothrix lutea, Diacamma pallidum, Haliporoides triarthrus, Dendroctonus pseudotsugae, Illinoia sp. BOLD-2016, Phelotrupes auratus, Tectocepheus sarekensis, Anochetus madagascarensis, Littorina obtusata, Suillia sp. BOLD-2016, Zanclognatha sp. BOLD: AAA5206, Neocalanus plumchrus, Botanophila profuga, Aphis pomi, Plagiolepis taurica, Lagocephalus spadiceus, Phyllocnistis vitegenella, Lycoriella secundaria, Chydorus environmental sample, Pyrgus armoricanus, Smittia sp. BOLD: AAB0375, Ascaris suum, Perca fluviatilis, Aphidius uzbekistanicus, Dinopsyllus ellobius, Hexagenia limbata, Leptinotarsa decemlineata, Marphysa corallina, Rivula propinqualis, Repomucenus filamentosus, Feniseca tarquinius, Rhipicephalus appendiculatus, Boloria chariclea, Cetonia aurata, Suillia longipennis, Sabacon cavicolens, Melanterius servulus, Dysodia sp. Janzen06, Periphyllus testudinaceus, Leptodactylus luctator, Apodemus peninsulae, Astralium rhodostomum, Calcinus seurati, Ametrida centurio, Styela plicata, Lasius pallitarsis, Drosophila subobscura, Decapterus macarellus, Spilogona trigonifera, Limnadopsis birchii, Calcinus elegans, Camponotus sp. BOLD: AAA2372, Gammarus solidus, Brevicoryne brassicae, Eulaema meriana, Cricotopus tibialis, Holcoponera strigata, Procladius sp. BOLD: AAG3918, Arion vulgaris, Sphacelodes vulneraria, Drosophila serido, Apiomorpha minor, Dipturus chilensis, Maniola cecilia, Wasmannia sp. MAS001, Temnothorax ambiguus, Raoiella indica, Aricia eumedon, Pseudoplatystoma fasciatum, Haliotis tuberculata, Carcharhinus sorrah, Parasaccocoelium mugili, Fulmarus glacialis, Dicyrtoma maculosa, Hyphantrophaga virilis, Tomicus piniperda, Pseudalmenus chlorinda, Megaselia fungivora, Xanthorhoe abrasaria, Parasaissetia nigra, Chaoborus cf. flavicans DJT-2005, Bathymodiolus marisindicus, Planiliza affinis, Parasteatoda tepidariorum, Phaonia sp. BOLD: AAC0888, Sperata lamarrii, Urbanus esmeraldus, Limnephilus nigriceps, Acronicta dactylina, Torymus flavipes, Neritina dilatata, Therioaphis trifolii, Microtus rossiaemeridionalis, Bombus vagans, Automeris sp. Janzen03, Palpita magniferalis, Pegomya sp. BOLD: AAG2506, Sweltsa lamba, Lauterborniella agrayloades, Ochlerotatus dorsalis, Idarnes carme, Adineta vaga, Lunella granulata, Ophisops elegans, Oncorhynchus gorbuscha, Zelotes fratris, Megaselia aristalis, Cratichneumon viator, Conus miliaris, Argiope trifasciata, Sarsia lovenii, Strongylocentrotus pallidus, Orthops palus, Euxoa comosa, Phyllonorycter maestingella, Dolichopus sp. SEB32, Conus ebraeus, Baicalia carinata, Rhizophagus dimidiatus, Gerres filamentosus, Fannia subpellucens, Alviniconcha hessleri, Hesperotettix viridis, Cicadula melanogaster, Toxocara cati, Asphondylia borrichiae, Setophaga coronata, Chironomus bifurcates, Helix pomatia, Cambarus longulus, Rhynchophorus palmarum, Rasbora pauciperforata, Caridina cantonensis, Hemerobius humulinus, Stenamma sp. BOLD-2016, Hypselistes florens, Procladius barbatus, Zelus luridus, Erebia aethiops, Coenosia pumila, Meloe coarctatus, Pheidole sp. MAS018, Pomatomus saltatrix, Cyclophora pendulinaria, Bathymodiolus nancyschneiderae, Cordyla sp. BOLD: ABU5545, Solea solea, Trimorus sp. BOLD-2016, Diamesa bertrami, Trypanoxyuris minutus, Lathrolestes thomsoni, Papilio rumiko, Scheloribates pallidulus, Culicoides actoni, Tachuda sp. discretaDHJ02, Elasmostethus cruciatus, Lycodon rufozonatus, Diplostomum baeri, Cyaniris semiargus, Carcharhinus brevipinna, Agelenopsis potteri, Iphiclides podalirius, Melitaea nevadensis, Erythroneura vitifex, Anopheles hyrcanus, Vatesus aff. goianus BOLD: ACW9450, Corynoptera saccata, Dugesia subtentaculata, Pontodrilus longissimus, Cinara formosana, Hypomicrogaster sp. Miii, Aporrectodea icterica, Ceraclea excisa, Simulium hirtipupa, Thunnus obesus, Blepharoneura sp. 8 MC-2007, Leptidea reali, Virbia ferruginosa, Chlorodiella laevissima, Rhinogobius similis, Atypoides riversi, Haplothrips sp. BOLD: AAG2812, Lophocampa maculata, Sympetrum danae, Stigmella salicis, Sergentomyia minuta, Xylosandrus germanus, Parnassius phoebus, Triops longicaudatus, Drosophila willistoni, Palpomyia sp. BOLD-2016, Hyalomma aegyptium, Acanthonyx lunulatus, Xestia imperita, Leptacis sp. BOLD-2016, Pegomya winthemi, Gymnogobius isaza, Clostera albosigma, Adenomera marmorata, Theclinesthes albocincta, Hipposcarus longiceps, Stenoma sp. Janzen07, Epomophorus pusillus, Panorpa liui, Eulecanium kuwanai, Hemiceras sp. zulaDHJ02, Automeris banus, Corethrella ranapungens, Sphyrna zygaena, Polyommatus ripartii, Branta canadensis, Arichanna melanaria, Bombus brasiliensis, Scaphytopius sp. BOLD-2016, Plagiognathus sp. BOLD-2016, Echinocladius martini, Meloidogyne hapla, Callobius nomeus, Urocitellus undulatus, Chionodes sp. BOLD-2016, Pampus punctatissimus, Blepharoneura sp. n. 1 Pe-2008, Ectropis obliqua, Maritrema novaezealandense, Tanytarsus dendyi, Didelphis albiventris, Essigella californica, Parabagrotis exsertistigma, Coelosia gracilis, Formica sp. BOLD: AAA1468, Diarsia rosaria, Coleomegilla maculata, Chrysotus sp. BOLD: ABY6054, Plateumaris sericea, Nannophya pygmaea, Hybomitra lurida, Bleptina inferior, Acanthopagrus latus, Brenthis ino, Dismodicus decemoculatus, Labeo bata, Uroteuthis duvaucelii, Anaphes listronoti, Callibaetis ferrugineus, Littoraria irrorata, Hydriomena sp. BOLD-2016, Culicoides victoriae, Simulium sp. BOLD: ACI0635, Gonioctena pallida, Culicoides wadai, Anochetus pubescens, Nymphalis c-album, Cicindela formosa, Rhamdia guatemalensis, Cryptochironomus psittacinus, Cicindela chinensis, Pseudodiaptomus forbesi, Chaetodon trifascialis, Hippocampus zosterae, Greya politella, Trixagus chevrolati, Sassia remensa, Ankylocythere sinuosa, Diplostomum sp. 4 SAL-2008, Metapenaeus monoceros, Anopheles albitarsis G Krzywinski et al., 2011, Andrias davidianus, Pyganodon grandis, Cartodere nodifer, Phengaris alcon, Leuciscus leuciscus, Argynnis paphia, Dugesia japonica, Barbus cyri, Cichla kelberi, Parasagitta elegans, Vampyressa bidens, Boloria pales, Culicoides punctatus, Ablabesmyia aspera, Montastraea cavernosa, Spargania magnoliata, Atrax 27 sutherlandi, Platycephalus indicus, Longpotamon yangtsekiense, Chilo orichalcociliellus, Fistularia commersonii, Pectenogammarus scutarensis, Coenobita purpureus, Planococcus ficus, Clitarchus tepaki x Clitarchus hookeri, Neochlamisus bebbianae, Heterodera glycines, Asterias amurensis, Oncorhynchus clarkii, Ochlerotatus hexodontus, Corticarina sp. BOLD-2016, Hypomesus olidus, Choristoneura occidentalis, Megalobrama terminalis, Ochlerotatus punctor, Syrphus ribesii, Troglocharinus ferreri, Rastrelliger brachysoma, Sinibotia robusta, Takifugu rubripes, Oxyethira serrata, Pollicipes pollicipes, Eukiefferiella claripennis, Epiphyas postvittana, Pseudosuccinea columella, Pheidole sp. MAS034, Glossosoma intermedium, Gyrodactylus arcuatus, Helicobia rapax, Copaxa moinieri, Pareledone aequipapillae, Mangora placida, Acanthis flammea, Konosirus punctatus, Protapanteles sp. BOLD-2016, Hyperoglyphe antarctica, Monochamus alternatus, Calcinus laevimanus, Biomphalaria pfeifferi, Parides ascanius, Issoria lathonia, Trixoscelis fumipennis, Simulium chumpornense, Penaeus brasiliensis, Eutrichota tunicata, Culex gelidus, Bungarus multicinctus, Centropomus undecimalis, Profilicollis altmani, Eratoneura sp. BOLD-2016, Lesmone formularis, Glyptapanteles sp. Whitfield141, Mansonia uniformis, Thrips hawaiiensis, Pseudotelphusa sp. BOLD-2016, Trematomus scotti, Polistes fuscatus, Spilosoma virginica, Tanytarsus gracilentus, Littoraria pallescens, Monacha claustralis, Biomphalaria occidentalis, Ochlerotatus cantans, Pigritia sp. n. P7, Ancylostoma ceylanicum, Agabus sturmii, Chalarus decorus, Catharus guttatus, Australapatemon burti complex sp. LINI, Limnephilus picturatus, Trachelipus rathkii, Isomira quadristriata, Tomosvaryella sp. BOLD-2016, Telphusa sedulitella, Cicindela lusitanica, Symbion sp. MO-2005, Spinturnix myoti, Paratanytarsus dissimilis, Nanophyes marmoratus, Eristalis tenax, Elaphe carinata, Pasiphila rectangulata, Coxiella glauerti, Homorthodes furfurata, Corynoptera deserta, Tanytarsus volgensis, Penthetria heteroptera, Steromphala umbilicaris, Eulithis xylina, Erigone arctica, Dahlica lichenella, Hypomicrogaster sp. Whitfield08,Hypomicrogaster sp. Whitfield08, Polycelis coronata, Monacha cartusiana, Siphonella oscinina, Chiroderma villosum, Archivesica gigas, Ammodytes personatus, Megalurothrips usitatus, Pheidole pugnax, Bicellaria furcifer, Tetrastichus halidayi, Leptotrombidium scutellare, Megaselia giraudii, Stenomacrus nemoralis, Engraulis japonicus, Parasagitta setosa, Grapholita funebrana, Panopeus herbstii, Oryctes borbonicus, Exechia frigida, Collichthys lucidus, Satsuma eucosmia, Cephalopholis urodeta, Limotettix arctostaphyli, Gymnopternus sp. BOLD-2016, Subpsaltria yangi, Maconellicoccus hirsutus, Epitrimerus sabinae, Chymomyza amoena, Amphioctopus aegina, Parasesarma bidens, Limnoperna fortunei, Culicoides fulvus, Phaonia serva, Parasa consocia, Taenia krabbei, Prosimulium arvum, Trirhabda borealis, Ctenocephalides orientis, Falagonia mexicana, Nystalea sp. moronaDHJ02, Agelasa nigriceps, Simenchelys parasitica, Toxascaris leonina, Hybauchenidium aquilonare, Pimelodus grosskopfii, Dictyna brevitarsa, Condylostylus sp. BOLD-2016, Cynopterus sphinx, Culex pallidothorax, Xylophanes sp. Janzen04, Anthicus cervinus, Cenolia sp. B KMN-2013, Tetradonia marginalis, Hemidactylus frenatus, Tricholochmaea cavicollis, Nemipterus japonicus, Barbatula barbatula, Pelegrina galathea, Amynthas corticis, Rhagada elachystoma, Blepharoneura sp. 1 MC-2007, Alysia sp. jft01, Ula sylvatica, Lepus californicus, Cherax quadricarinatus, Centropages typicus, Colocasiomyia xenalocasiae, Gigantidas platifrons, Cynopoecilus melanotaenia, Pontia edusa, Nysius sp. BOLD-2016, Acropora hyacinthus, Oecophylla smaragdina, Aprostocetus sp. BOLD-2016, Pagrus pagrus, Astroides calycularis, Glyptapanteles sp. Whitfield71, Agroeca ornata, Tanytarsus wirthi, Pseudopimelodus atricaudus, Coenipeta medina, Elachista sp. BOLD-2016, Tricimba sp. BOLD-2016, Anguispira kochi, Stenomacrus sp. BOLD: AAF3467, Sulcascaris sulcata, Limnophyes sp. BOLD: AAB1810, Rachiplusia nu, Schizothorax richardsonii, Megaselia sp. 49 LL-2023a, Glossina palpalis, Chytolita morbidalis, Diuraphis noxia, Semaeopus sp., Branchipus schaefferi, Chauliodus sloani, Tydeus sp. BOLD: AAH3904, Rhamphomyia sp. BOLD: AAP2854, Blepharoneura sp. 28 MC-2007, Miltochrista miniata, Rheumaptera hastata, Eupithecia lariciata, Pennella sp. (in: crustaceans), Spheniscus demersus, Uroteuthis chinensis, Ooctonus hemipterus, Scarus ghobban, Lipaphis pseudobrassicae, Trogoderma granarium, Lispe cotidiana, Epinotia cruciana, Plexippus paykulli, Anopheles atroparvus, Kryptolebias ocellatus, Euthynnus alletteratus, Ranoidea rheocola, Xysticus benefactor, Plagiodera versicolora, Ctenopharyngodon idella, Chaetodipterus faber, Corynoneura sp. 3ES, Helina pectinata, Stolephorus indicus, Choristoneura pinus, Zygothrica sp. 41DG, Trochulus striolatus, Cottus aleuticus, Trichiurus japonicus, Spelobia sp. BOLD-2016, Utetes canaliculatus, Megaselia sp. 54 LL-2023a, Platycheirus quadratus, Pipistrellus kuhlii, Artemia salina, Megaselia nigriceps, Cooperia oncophora, Taeniothrips orionis, Lysiphlebus fabarum, Brenthis daphne, Capra hircus, Phyllonorycter salictella, Thricops albibasalis, Tupiocoris sp. BOLD-2016, Anneissia benhami, Pseudolaubuca engraulis, Parastromateus niger, Platypleura kaempferi, Serranus cabrilla, Anodonta nuttalliana, Macaria submarmorata, Talides sp. Burns02, Chanodichthys erythropterus, Zonotrichia albicollis, Dikerogammarus villosus, Mycetophila uninotata, Pholetesor ornigis, Myriapora truncata, Meimuna oshimensis, Ctenocalanus citer, Epomophorus cf. labiatus DMR-2017, Panthea virginaria, Scomberomorus commerson, Ixodes persulcatus, Ictalurus punctatus, Pococera aplastella, Erythroneura sp. BOLD: ABA5798, Capitella nonatoi, Ophiactis savignyi, Costasiella ocellifera, Peromyscus leucopus, Hylesia continua, Rothschildia lebeau, Lepetodrilus schrolli, Odontotaenius disjunctus, Caridina indistincta A, Catharus ustulatus, Megaselia variana, Cephaloleia dilaticollis, Hexechamaesipho pilsbryi, Bagrada hilaris, Hydroporus sp. HPE14, Serrasalmus rhombeus, Poecilia reticulata, Hemiceras vecina, Thelazia callipaeda, Leptuca pugilator, Lysandra bellargus, Megaselia sp. 50 LL-2023a, Apostichopus japonicus, Ophiocomina nigra, Tor putitora, Euura sylvestris, Asteromyia sp. BOLD-2016, Asterias rubens, Nylanderia sp. 24 of SKY, Trochosa ruricola, Stenopsyche marmorata, Caranx sexfasciatus, Neanthes kerguelensis, Herpetogramma thestealis, Ceriodaphnia cornuta, Cicadula subcupraea, Diphthera festiva, Macrobrachium americanum, Mythimna monticola, Sardinella aurita, Megaselia longicostalis, Cheyletus malaccensis, Paraleptophlebia mollis, Lasioglossum calceatum, Odontotermes formosanus, Uroteuthis edulis, Rhopalosiphum nymphaeae, Aphonopelma mojave, Hyla molleri, Chrysops furcatus, Scathophaga suilla, Anastrepha suspensa, Lasioglossum sp. BOLD-2016, Isotomiella minor, Anthocoris musculus, Spilogona sp. BOLD: ABZ0955, Diaphanosoma environmental sample, Planococcus minor, Atractodes sp. BOLD: AAC7770, Errastunus ocellaris, Microtus pennsylvanicus, Smerinthus cerisyi, Triplophysa stenura, Desmodus rotundus, Daphnia galeata, Lestodiplosis sp. 1 NPH-2020, Argyresthia oreasella, Pachycondyla sp. JTL014, Millepora platyphylla, Millepora exaesa, Dastarcus helophoroides, Psectrocladius sp. BOLD-2016, Vandiemenella viatica, Stenamma brevicorne, Barbus barbus, Hoplostethus atlanticus, Anaphes sp. BOLD-2016, Hoplopleura patersoni, Acrocephalus scirpaceus, Pterygoplichthys pardalis, Morpho helenor, Chazara briseis, Apamea amputatrix, Abyssogena southwardae, Psammotermes allocerus, Clepsis peritana, Bactrocera tsuneonis, Boletina palmata, Acheta domesticus, Stigmella sp. BOLD-2016, Paracalanus quasimodo, Gouania adriatica, Liriomyza fricki, Palthis angulalis, Batrachyla leptopus, Drosophila albomicans, Excirolana natalensis, Ceratina calcarata, Girardia sp. C KI-2020, Autographa precationis, Lehmannia marginata, Sylvicola sp. BOLD-2016, Ectemnorhinus similis, Lipoptena cervi, Sicydium buscki, Cosmosoma hercyna, Euxoa tritici, Clethrionomys gapperi, Hydrotaea sp. BOLD-2016, Alpheus angulosus, Aphis varians, Oxyopes hindostanicus, Junonia genoveva, Chrysaora melanaster, Caprella penantis, Nipponoluciola cruciata, Banasa dimiata, Melampus jaumei, Syngnathus schlegeli, Ligia cinerascens, Centromerus sylvaticus, Gavialis gangeticus, Cricotopus sp. BOLD: AAG1016, Apamea zeta, Radix cf. plicatula TS-2021 Clade VI, Lasius niger, Meloidogyne incognita, Araneus omnicolor, Elaphria versicolor, Rutilus rutilus, Potamonautes sidneyi, Rhombognathus levigatoides, Parnassius mnemosyne, Selenops lindborgi, Radix rubiginosa, Paracalanus parvus, Sisicottus nesides, Platycheirus hyperboreus, Cryptocotyle lingua, Timema poppensis, Bradysia scabricornis, Blepharipa albicauda, Chironomus sp. TE11, Culicoides mahasarakhamense, Megaselia sp. BOLD: ABU5529, Glyptapanteles sp. Whitfield121, Portunus sanguinolentus, Clupea pallasii, Dorcus montivagus, Monomorium sp. 1 of SKY, Synodontis schall, Exosoma lusitanicum, Ephemera orientalis, Phaonia apicalis, Chelifera frigelii, Rhinichthys obtusus, Perognathus flavus, Amoimyrmex striatus, Coccophagus japonicus, Polypsocus corruptus, Euschistus servus, Pseudophilotes vicrama, Hesperia comma, Lissotriton helveticus, Cheumatopsyche campyla, Scomberomorus plurilineatus, Patiria pectinifera, Cacopsylla pyricola, Pollachius virens, Dendropoma petraeum, Auxis thazard, Anopheles funestus, Wallago attu, Aegla platensis, Arichanna perimelaina, Hemiceras transducta, Tetragnatha sp. A JC-2014, Tanytarsus sp. BOLD: AAG5529, Pocillopora damicornis, Taenia saginata, Rhingia nasica, Scinax fuscomarginatus, Nephrotoma cornicina, Leptus sp. BOLD: AAU6288, Ablabesmyia illinoensis, Rhagio sp. BOLD: AAH2729, Hypomicrogaster rugosus, Trachyrhamphus serratus, Euhadra herklotsi, Ompok bimaculatus, Mytilopsis sallei, Dibothriocephalus dendriticus, Deronectes aubei, Drosophila immigrans, Acetes indicus, Amitermes laurensis, Meteorus trachynotus, Monoclona rufilatera, Tipula serta, Adelpha sp. basiloidesDHJ02, Anarta trifolii, Oppiella nova, Aeshna grandis, Drosophila mauritiana, Pinctada margaritifera, Dracunculus medinensis, Memphis forreri, Pheosia rimosa, Glypta fumiferanae, Cheumatopsyche halima, Liolophura japonica, Apamea sordens, Vespula alascensis, Clemensia albata, Handleyomys melanotis, Scheloribates clavilanceolatus, Phygadeuon sp. HJB-2017, Phyllognathopus aff. viguieri 1 SK-2020, Ravinia stimulans, Spelobia clunipes, Sciaphilus asperatus, Melospiza lincolnii, Satsuma largillierti, Hemirhamphodon pogonognathus, Vireo olivaceus, Scrobicularia plana, Kryptolebias marmoratus, Euseius nicholsi, Oestrus ovis, Longpotamon acutum, Biomphalaria sp. CJS-2010, Trixagus sp. BOLD: AAU6966, Mycetophila sp. BOLD: AAG4893, Saurida undosquamis, Inia boliviensis, Echinoderes sensibilis, Glyptothorax platypogon, Pardosa furcifera, Erythroneura sp. BOLD: ABA5871, Cladotanytarsus atridorsum, Metasiro americanus, Macridiscus multifarius, Rhamphomyia latilobata, Platypalpus sp. BOLD: AAP6357, Leiomyza curvinervis, Anaplectoides pressus, Diplocladius cultriger, Aulonothroscus constrictor, Trichordestra liquida, Boreonectes griseostriatus, Zelandobius confusus, Stigmella betulicola, Gobiopterus lacustris, Eucalliphora latifrons, Syncopacma sp. BOLD-2016, Alviniconcha strummeri, Erythridula sp. BOLD-2016, Austromegabalanus psittacus, Monarthrum scutellare, Listropsylla agrippinae, Sarcophaga praedatrix, Evynnis cardinalis, Styela canopus complex sp. RCB-2023, Palaemon argentinus, Adeloneivaia jason, Ototyphlonemertes fila, Carangoides malabaricus, Homidia sinensis, Leucopis hennigrata, Aliatypus janus, Scopelocheirus schellenbergi, Unedogemmula cf. indica NP-2008, Selenops insularis, Jaminia quadridens, Fabriciana niobe, Salurnis marginella, Takifugu xanthopterus, Hippodamia variegata, Ptilodexia conjuncta, Hydroporus sp. HPE31, Plectropomus leopardus, Hemiculter bleekeri, Risor ruber, Oligolophus tridens, Alopias superciliosus, Palaemon suttkusi, Takydromus septentrionalis, Anax junius, Cryptolestes pusillus, Anopheles dunhami, Mellicta athalia, Pelegrina proterva, Ophiothrix sp. RPP-2012a, Branchipolynoe halliseyae, Atta sexdens, Pseudorthocladius sp. BOLD-2016, Xestia homogena, Hormogaster redii, Minuca pugnax, Haematobia irritans, Agriotes fucosus, Schizura unicornis, Dicentria sp. violascensDHJ01, Cephaluris alaskensis, Euura sp. BOLD-2016, Carterocephalus palaemon, Euxoa divergens, Stenoma sp. Janzen19, Ophion kevoensis, Caryocolum pullatella, Macoma calcarea, Nabis americoferus, Pomphorhynchus tereticollis, Microscolex phosphoreus, Hexaplex trunculus, Aphis nerii, Neogobius melanostomus, Phronia sp. BOLD-2016, Lineus ruber, Drosophila takahashii, Calopteryx maculata, Unio tumidus, Hemeroplanis incusalis, Psilocorsis reflexella, Cordulegaster sp. BOLD: AAB2922, Microcalanus sp. A AC-2021, Drepana arcuata, Lepidocephalichthys guntea, Romualdius bifoveolatus, Limnophyes sp. 11ES, Calaphis flava, Potamotrygon motoro, Propylea quattuordecimpunctata, Ochlerotatus taeniorhynchus, Boreomysis nobilis, Eulaema cingulata, Phrurotimpus borealis, Coenonympha arcania, Scathophaga sp. BOLD: AAD0853, Aoraki denticulata, Cryptotympana facialis, Phelsuma andamanense, Papilio cresphontes, Mustelus punctulatus, Panurgus dentipes, Acar gradata, Leptotes pirithous, Dalopius sp. BOLD: AAL8068, Garra rufa, Coreius guichenoti, Carabus jankowskii, Scolopendra subspinipes, Hercostomus unicolor, Eupithecia miserulata, Hemideina thoracica, Nystalea sp. collarisDHJ13, Devario aequipinnatus, Sepsis neocynipsea, Gyrodactylus salaris, Sicydium punctatum, Strauzia longipennis, Draculoides kryptus, Tylos granuliferus, Vespula pensylvanica, Tetrapus sp., Metriocnemus sp. 4ES, Pygospio elegans, Megapalpus capensis, Vespula maculifrons, Leptoglossus zonatus, Odoiporus longicollis, Ectatomma ruidum, Pheidole rugiceps, Osmerus mordax, Geomalacus anguiformis, Hypanus guttatus, Vitreorana uranoscopa, Hypaurotis quercus, Sturnira ludovici, Hyperomyzus lactucae, Formica dakotensis, Synaphobranchus kaupii, Chrysoperla nipponensis, Colocasiomyia alocasiae, Astraptes enotrus, Gammarus nekkensis, Bucculatrix sp. BOLD: AAD3127, Dysstroma citrata, Aproaerema anthyllidella, Bibio sp. BOLD: AAG6649, Contacyphon sp. BOLD-2016, Pyrocoelia rufa, Coenosia comita, Panulirus japonicus, Cnaphalocrocis medinalis, Octopus pallidus, Asynarchus mutatus, Blepharoneura sp. 2 MC-2007, Lygus pratensis, Cephaloleia congener, Anopheles cruzii, Arenivaga floridensis, Paracolax tristalis, Piletosoma sp. thialisDHJ03, Hyalomma marginatum, Dermogenys collettei, Limnocalanus macrurus, Lampsilis dolabraeformis, Pampus minor, Tribelos sp. BOLD: AAG0920, Allolobophora chlorotica complex sp. L1, Crassostrea hongkongensis, Chaetodipus arenarius, Tetanocera sp. BOLD: AAD2073, Echinarachnius parma, Paratrechina longicornis, Deroceras reticulatum, Drosophila buzzatii, Polydrusus cervinus, Drymeia sp. BOLD: AAD7664, Corythucha sp. BOLD: AAG8847, Craspedolepta sp. BOLD: AAF9329, Fortuynia elamellata, Murina recondita, Sicya macularia, Trachops cirrhosus, Tanytarsus herrmanni, Scaptomyza adusta, Scirpophaga incertulas, Pteronotus personatus, Choreutidae gen. chorJanzen01 sp. Janzen01, Pteronotropis euryzonus, Agnippe leuconota, Apodemus agrarius, Murshidia linstowi, Pagurus samuelis, Gerres oyena, Catopocerus sp., Boloria euphrosyne, Asynapta groverae, Parachanna obscura, Lobophora nivigerata, Alectorobius capensis, Aulacorthum solani, Hippocampus kuda, Peripatopsis clavigera, Culex theileri, Erebia ligea, Phyciodes cocyta, Platygaster sp. BOLD-2016, Turbo sazae, Simulium silvestre, Lophogobius cyprinoides, Drusus croaticus, Apanteles sp. Rodriguez06, Floridichthys polyommus, Nasutitermes ephratae, Colias sareptensis, Trichogramma sp., Hipparchia semele, Idiocerus formosus, Rhodnius prolixus, Simulium weji, Aliatypus thompsoni, Heteractis magnifica, Coquillettidia richiardii, Luehdorfia puziloi, Caenurgina crassiuscula, Bradybaena phaeogramma, Mustelus mustelus, Turtonia minuta, Sperata aor, Hyalomma anatolicum, Lymantria monacha, Arcuatula senhousia, Dysmicoccus neobrevipes, Drosophila bipectinata, Anopheles claviger, Necora puber, Simulium atratum, Maurolicus muelleri, Mytilopsis leucophaeata, Metriocnemus sp. 1ES, Anabas testudineus, Nerita atramentosa, Penicillidia fulvida, Smaragdina concolor, Ophiogomphus mainensis, Theridion differens, Culicoides montanus, Tomocerus sp. BOLD: AAG5019, Monomorium floricola, Meteorus pulchricornis, Sphaerophoria sp. BOLD: AAA7374, Artibeus intermedius, Coproica ferruginata, Caloptilia fraxinella, Pelegrina montana, Chionodes sattleri, Helix lucorum, Leptuca thayeri, Gonepteryx cleopatra, Triphleba sp. BOLD-2016, Artemia tibetiana, Euplectrus sp. CHEUPO1, Neolucanus doro, Psychomyia pusilla, Hydroides dianthus, Xestocephalus desertorum, Polyeunoa laevis, Harmothoe imbricata, Triplophysa nujiangensa, Boreonectes striatellus, Sturnira luisi, Tachyporus nitidulus, Paralacydonia weberi, Euscelidius variegatus, Parametriocnemus boreoalpinus, Limnephilus sansoni, Proasellus coxalis, Nemopilema nomurai, Lepidocyrtus sp. BOLD: ACL5869, Asynarchus rossi, Pocadicnemis pumila, Anagrus sp. BOLD: AAN8044, Pardosa dorsuncata, Paroster mesosturtensis, Lagopus lagopus, Cryptozona siamensis, Gammarus duebeni, Schizothorax wangchiachii, Trachinocephalus myops, Brachidontes puniceus, Bathymodiolus azoricus, Gonipterus platensis, Helobdella stagnalis, Clubiona pallidula, Hybos reversus, Paratanytarsus sp. 1ES, Dasyhelea modesta, Lascoria ambigualis, Scoparia sp. BOLD: AAA8364, Selatosomus pulcher, Improphantes complicatus, Scutellonema bradys, Telenomus sp. BOLD: AAN8098, Metopolophium dirhodum, Hypomecis punctinalis, Zerynthia polyxena, Neodexiopsis calopyga, Catopsilia pomona, Coenocorypha aucklandica, Bombus cryptarum, Orchesella villosa, Abyssorchomene gerulicorbis, Xylocopa darwini, Culex pseudovishnui, Diadema setosum, Elona quimperiana, Simulium mediovittatum, Encrasicholina punctifer, Psilogramma menephron, Lutjanus campechanus, Carcharhinus leucas, Publilia concava, Tanytarsus sp. BOLD: AAP5870, Metriocnemus eurynotus, Colias palaeno, Carollia sowelli, Phalacrodectes gaudi, Melanolophia imitata, Sweltsa borealis, Clubiona kulczynskii, Pieris mannii, Ceratina dupla, Anopheles punctulatus, Cerithideopsis californica, Trematomus eulepidotus, Polyommatus dorylas, Culex univittatus, Araptus attenuatus, Polythrix sp. Janzen02, Pyroteuthis margaritifera, Chrysops ater, Pseudosmittia sp. BOLD: AAL9425, Halictus rubicundus, Acerpenna sp. AMI 1, Stylochoplana pusilla, Bungalotis sp. quadratumDHJ01, Bombus occidentalis, Odontesthes argentinensis, Lomographa vestaliata, Eadya sp. 3 BJS-2017, Callogorgia delta, Aphidius urticae, Spilogona contractifrons, Culicoides sp. BOLD: ABV1155, Hippodamia parenthesis, Helophilus hybridus, Bibio longipes, Phyllostomus discolor, Azumapecten farreri, Schizocosa ocreata, Leucrocuta hebe, Incertella minor, Oreina alpestris, Cinara laricifex, Epinotia solicitana, Hypophthalmichthys nobilis, Mesocriconema nebraskense, Anastrepha ludens, Thylacodes variabilis, Contarinia sp. n. BAM-2018, Cisseps fulvicollis, Phengaris arion, Hemibagrus nemurus, Forcipomyia taiwana, Dolichogenidea sp. jft03, Curtonida isos, Brachyderes rugatus, Platypalpus stabilis, Haliplus sp. HAE9, Empoasca sp. BOLD: ACF5026, Lachnaia hirta, Agabus sp. AGE20, Anastrepha distincta, Drosophila denieri, Pampus chinensis, Pityohyphantes limitaneus, Glaucus atlanticus, Trichogramma brassicae, Ecitophora cf. comes 1 CVB-2021a, Etheostoma radiosum, Aphis rubicola, Exoneurella setosa, Hylaeamys megacephalus, Idiocerus lachrymalis, Maccaffertium modestum, Cerion incanum, Pyrgulopsis kolobensis, Elachista sp. BOLD: AAD9052, Excirolana latipes, Oxidercia sp. thaumantisDHJ02, Eumunida annulosa, Heterocampa guttivitta, Rivula sp. Janzen40, Plotosus lineatus, Telegonus anaphus, Naupactus xanthographus, Bibarrambla allenella, Myospila meditabunda, Microplitis sp. jft16, Eacles sp. Janzen01, Pyrgus malvoides, Leucinodes africensis, Euxoa tessellata, Maliattha synochitis, Simulium nigrogilvum, Odontomachus coquereli, Carcharhinus limbatus, Zaphne lineatocollis, Fregatiella aurifasciata, Mesopsocus unipunctatus, Araneus diadematus, Bombus hypocrita, Garella nilotica, Cryptocephalus octoguttatus, Autographa pulchrina, Telenomus sp. BOLD: ACB8863, Desmia sp. Solis100DHJ01, Cricotopus sp. 18ES, Hydra vulgaris, Platycerus takakuwai, Badis badis, Sepia pharaonis, Dicyrtomina sp. BOLD: AAF5859, Simulium saxosum, Anopheles daciae, Mecaphesa asperata, Macrochlamys indica, Rimicaris exoculata, Ophiacantha vivipara, Platypalpus nr. albiseta BOLD: ACC4222, Nehalennia irene, Dugesia benazzii, Megaselia sp. 65 LL-2023a, Hydropsyche fimbriata, Apanteles sp. Rodriguez51, Nephelodes minians, Ferrisia virgata, Pleurocera prasinata, Pennahia argentata, Caloptilia stigmatella, Cellana talcosa, Melanophthalma sp. BOLD: AAM7680, Hylaeus annulatus, Niphargus valachicus, Lineus viridis, Dynastes hercules, Ishizakiella miurensis, Haustorioides japonicus, Typhlocyba gillettei, Salvelinus malma, Glauce pectenalaeella, Micropterus salmoides, Anaxyrus boreas, Ampelophaga rubiginosa, Pygopleurus foina, Rhynchophorus vulneratus, Cellana sandwicensis, Polyplax arvicanthis, Pycna semiclara, Sebastes miniatus, Ligidium sp., Essigella sp. BOLD-2016, Kamimuria tibialis, Aeolidia papillosa, Camptocladius stercorarius, Maniola hyperantus, Cratyna ambigua, Naso hexacanthus, Tomocerus minor, Pagellus erythrinus, Atractodes sp. BOLD: ABZ2717, Lygus keltoni, Barbonymus gonionotus, Rhagoletis pomonella, Entomobrya albocincta, Bucculatrix sp. BOLD: AAB1986, Junco phaeonotus, Agria affinis, Brachionus dimidiatus, Moolgarda perusii, Leucoma salicis, Amaurobius borealis, Paracirrhites forsteri, Coenosia attenuata, Acanthurus triostegus, Morulina thulensis, Nephila pilipes, Erynnis tages, Arctosa insignita, Hyalinobatrachium fleischmanni, Mystacides interjectus, Carybdea brevipedalia, Echiniscus blumi, Capoeta damascina, Grammonota gigas, Rhinolophus pearsonii, Sillago japonica, Chrysoperla carnea, Pulex irritans, Euproctis chrysorrhoea, Littorina saxatilis, Nematocampa resistaria, Rimicaris chacei, Tachysurus fulvidraco, Lucoppia burrowsi, Maoricicada campbelli, Camponotus (Tanaemyrmex) sp. 48 of SKY, Pediasia trisecta, Deladenus proximus, Miobantia fuscata, Sciophila sp. BOLD-2016, Dreissena polymorpha, Gobiosoma bosc, Sphyrapicus varius, Copaxa rufinans, Virbia aurantiaca, Cryptocala acadiensis, Oeneis jutta, Rhopilema nomadica, Phlebotomus tobbi, Oceanodroma melania, Ozyptila praticola, Nemoura arctica, Corynoneura sp. BOLD: AAN5330, Boletina sp. BOLD: ABZ2061, Trissolcus japonicus, Forficula aff. auricularia B BOLD-2015, Oulema melanopus, Acutuncus antarcticus, Puntius chelynoides, Mullus surmuletus, Lytechinus variegatus, Acanthodiaptomus pacificus, Limnephilus moestus, Culaea inconstans, Anopheles neivai, Eupithecia satyrata, Stenodema trispinosum, Oligonychus punicae, Chorocaris sp. 2 ADT-2013, Synapte silius, Ommatissus lybicus, Rattus argentiventer, Dichomma dardanum, Coelopa frigida, Epinephelus awoara, Ceratagallia sp. BOLD-2016, Tomicodon petersii, Xestocephalus superbus, Ichneumonidae gen. ichJanzen01 sp. Janzen01, Evadne nordmanni, Bombus sp., Dendrolimus kikuchii, Mermessus trilobatus, Longitarsus cerinthes, Stereophaedusa japonica, Amblyomma ovale, Watersipora subtorquata, Lepomis megalotis, Chelobasis bicolor, Nassarius nitidus, Artibeus phaeotis, Boletina minuta, Platycheirus sp. BOLD-2016, Oxytorus antennatus, Lamna nasus, Pediculus humanus complex sp. A MA-2014, Oxyopes azhari, Lissachatina fulica, Orthotaenia undulana, Penaeus kerathurus, Pagurus granosimanus, Zeuzera coffeae, Nephotettix virescens, Lutzomyia ayacuchensis, Saliana fusta, Limnophora uniseta, Obesogammarus crassus, Orthocladius sp. BOLD: ADR6362, Crepidodera aurata, Lepomis gibbosus, Ciona savignyi, Piabina argentea, Setophaga magnolia, Chlorodiella sp. 2 RML-2022a, Sisicottus montanus, Orchestes alni, Calliptamus italicus, Theretra oldenlandiae, Micaelamys namaquensis, Lygus sp. BOLD: ABZ1405, Limnephilus sericeus, Idiocerus xanthiops, Neotrypaea japonica, Calosoma granatense, Hysteroneura setariae, Branta hutchinsii, Olethreutes baccatana, Oreina elongata, Anas platyrhynchos, Haemaphysalis qinghaiensis, Saccostrea palmula, Caridina natalensis, Oreochromis amphimelas, Xestocephalus sp. BOLD: AAN8347, Phytomyza verticillatae, Ceratapion basicorne, Aphrodes makarovi, Desoria grisea, Branchiostoma japonicum, Botanophila fugax, Geraeocormobius sylvarum, Sarcophaga misera, Clubiona trivialis, Ceratina smaragdula, Paederus fuscipes, Bicellaria sp. BOLD-2016, Leuctra fusca, Exechia sp. BOLD-2016, Argynnis hesperis, Lutjanus johnii, Bentholebouria aff. georgenascimentoi lineage 2 ZL-2015, Maxomys surifer, Serratella ignita, Amblyseius swirskii, Ara macao, Dryas iulia, Empoasca sp. BOLD: AAV0226, Pomacea occulta, Smicridea mucronata, Dermestes maculatus, Cheumatopsyche sp. BOLD: AAA5695, Penaeus japonicus, Coleophora sp. BOLD: AAA3993, Tetradonia lizonae, Cryphoeca exlineae, Desmognathus monticola, Scophthalmus maximus, Trachurus mediterraneus, Empoasca sp. BOLD: AAN8289, Adhemarius eurysthenes, Palaemon macrodactylus, Ectropis grisescens, Limnophora rotundata, Hemirhamphodon sp. MDB-2013, Mycodiplosis sp., Parathelphusa maculata, Strauchbufo raddei, Lanthus parvulus, Chionarctia nivea, Udea rubigalis, Pseudaspius hakonensis, Crocidura olivieri, Amorphochelus retusus, Conchapelopia telema, Proechimys guyannensis, Melitaea phoebe, Vireo solitarius, Leiobunum aldrichi, Cittarium pica, Hippotion celerio, Phyllocnistis sp. BOLD-2016, Mytilus coruscus, Eudyptes chrysolophus, Pachygrapsus marmoratus, Galaxias zebratus, Ochlerotatus punctor subgroup sp. BOLD-2016, Quadrula quadrula, Ascaridia galli, Rhagada minima, Peleteria setosa, Micropsectra_logani, Rhynchonycteris naso, Gibbaranea occidentalis, Tridacna elongatissima, Carystoides sp. Burns01, Paralithodes camtschaticus, Halobates sobrinus, Teloganopsis deficiens, Rheocricotopus robacki, Arhodia lasiocamparia, Isotomurus cf. palustris MM-2005, Hydrobaenus conformis, Idas iwaotakii, Gryllus bimaculatus, Metriocnemus brusti, Polythore procera, Paragus haemorrhous, Plesiastrea versipora, Echinococcus shiquicus, Tachinus sp. BOLD: AAN6186, Gymnophora fastigiorum, Stylodrilus heringianus, Aporrectodea caliginosa L3, Schizohelea leucopeza, Elachista cucullata, Chlorospingus ophthalmicus, Unio foucauldianus, Cricotopus sp. 6ES, Cybister tripunctatus, Epidromia sp. pannosaDHJ02, Conus sanguinolentus, Patella ferruginea, Hemiceras nigrescens, Erioptera sp. BOLD-2016, Antaeotricha sp. radicalisEPRO3, Pteromalus sp. BOLD-2016, Eana osseana, Habronattus americanus, Boops boops, Xylosandrus crassiusculus, Pseudopachychaeta ruficeps, Thera juniperata, Leucauge venusta, Rhagada angulata, Chthamalus fissus, Herpetogramma salbialis, Platycerus delicatulus, Maackia herderiana, Zosterops japonicus, Cherokia georgiana, Gelis sp. BOLD-2016, Cratichneumon culex, Enantia mazai, Dysstroma walkerata, Bathymodiolus brooksi, Hydropsyche sp. BOLD: AAA1669, Lygus elisus, Merlangius merlangus, Aphonopelma hentzi, Polydrusus impressifrons, Micratya cooki, Melanargia russiae, Ozestheria cf. berneyi N MS-2015, Limnephilus femoralis, Paramuricea biscaya, Heterodera schachtii, Sarcophaga taenionota, Alcippe hueti, Synopeas myles, Spilogona tundrae, Maizania wahlbergi, Nothrus silvestris, Streptopelia decaocto, Sebastes mystinus, Choristoneura fractivittana, Pseudosciaphila duplex, Tarebia granifera, Automeris belti, Mycobates sp. BOLD: AAC6787, Laelaps agilis, Molanna flavicornis, Gammarus ochridensis, Bombus sylvicola, Pholetesor sp. BOLD-2016, Ophisma tropicalis, Macrocentrus cingulum, Microchironomus tener, Netuma thalassina, Eriocrania sangii, Pterostichus melanarius, Stictopleurus punctiventris, Anopheles lindesayi, Spherillo dorsalis, Niphargus hrabei, Orthocladius rivulorum, Cyrtocarenum cunicularium, Ablabesmyia monilis, Torquigener flavimaculosus, Scirtothrips aurantii, Scaptomyza flava, Xestia xanthographa, Afronurus hyalinus, Alepes kleinii, Gouania pigra, Iberus gualtieranus, Bythinella pannonica, Bucculatrix pomifoliella, Fusconaia askewi, Hypoprepia fucosa, Zeus faber, Choristoneura occidentalis group BOLD: AAA1517, Cebus albifrons, Bagarius bagarius, Maccaffertium sp. AMI 1, Calosoma sycophanta, Cephalopholis argus, Asota plana, Helictopleurus neoamplicollis, Hypsiboas sp. WCF-2012, Aleurodicus rugioperculatus, Epinephelus fasciatus, Setophaga palmarum, Grammonota gentilis, Tetragonula hockingsi, Sitona hispidulus, Ascotis selenaria, Phalaenophana pyramusalis, Spilogona griseola, Eupeodes sp. BOLD: AAB2384, Ilybius sp. ILE13, Blepharipa sp. fimbriataDHJ04, Anaticola crassicornis, Orthetrum coerulescens, Heliconius hermathena, Spelobia tufta, Rhabdomys dilectus, Diopatra neapolitana, Molossus rufus, Dysodia sp. Janzen01, Exochus pictus, Halozetes fulvus, Astrotoma agassizii, Ensis macha, Drosophila sp. BOLD: AAG8493, Solanometra antarctica, Unio delphinus, Copris tripartitus, Doryteuthis pleii, Lasius claviger, Proctophyllodes sylviae, Lirimiris inopinata, Microthyris sp. prolongalisDHJ02, Luscinia svecica, Ceratina australensis, Philygria debilis, Acropyga acutiventris, Paranemertes peregrina, Scatophagus argus, Iberorhyzobius rondensis, Leptorhynchoides thecatus, Garra mullya, Ilybius fuliginosus, Seladonia confusa, Trechus brembanus, Lamprochernes chyzeri, Bombina variegata, Papilio xuthus, Lutjanus fulviflamma, Zapada cinctipes, Sandelia capensis, Leuctra major, Lacinipolia lorea, Euploea alcathoe, Rhamphomyia sp. BOLD: ABY3, Rhamphomyia sp. BOLD: ABY3064, Oncorhynchus masou, Platycheirus scambus, Geophagus altifrons, Nyctoporis carinata, Telenomus sp. BOLD: AAG7991, Helice tientsinensis, Apteromyia claviventris, Habia rubica, Polythrix sp. Janzen01, Erythroneura sp. BOLD-2016, Lemmus trimucronatus, Diarsia rubifera, Aethes biscana, Phobocampe bicingulata, Iseropus stercorator, Sitochroa verticalis, Gammarus alpinus, Xanthochorema caledon, Physella ancillaria, Cybister brevis, Theba pisana, Zygaena filipendulae, Araneus saevus, Baetis brunneicolor, Ischnus sp. BOLD-2016, Asobara sp. BOLD-2016, Bythinella sp. GA, Longitarsus dorsalis, Tigriopus californicus, Phoebis sp. arganteDHJ01, Radix sp. clade 9 CC-2016, Aquatica lateralis, Limenitis arthemis, Cephaloplectus mus, Hydropsyche siltalai, Platichthys flesus, Polia nimbosa, Syngrapha octoscripta, Rhopalum clavipes, Campoletis flavicincta, Ranoidea serrata, Mecopoda niponensis, Eupithecia absinthiata, Hydatigera taeniaeformis, Agriphila sp. BOLD: AAA3920, Dahlica sp. BOLD: AAD1523, Oreohelix cooperi, Meragisa sp. mimicaDHJ01, Noricella oreinos, Abida secale, Channa harcourtbutleri, Paratanytarsus natvigi, Cynea cynea, Pityohyphantes cristatus, Heringia calcarata, Udranomia sp. kikkawaiDHJO1, Idia lubricalis, Phascolosoma scolops, Siphlonurus alternatus, Bathyraja smirnovi, Plagodis pulveraria, Lutjanus griseus, Enteromius pellegrini, Hesperophylax designatus, Podabrus rugosulus, Pontia daplidice, Antaeotricha sp. Janzen224, Adhemarius gannascus, Megaselia sp. BOLD: AAL9076, Leporinus friderici, Limnoria nagatai, Rattus exulans, Dicrostonyx richardsoni, Orthoperus scutellaris, Siphosturmia sp. rafaeliDHJ03, Regulus satrapa, Thaumatomyia pulla, Melanoplus sanguinipes, Berberomeloe majalis, Echinometra sp. EZ_OB-2013, Phreagena soyoae, Trachyopella nuda, Cupido argiades, Hydrellia notata, Philodromus dispar, Taeniogonalos sp. RAINFORESTO, Orconectes australis, Ocyurus chrysurus, Ancylus sp. Cl, Photinus pyralis, Panthera tigris, Pardosa uintana, Olibrus semistriatus, Heteromys gaumeri, Euceraphis borealis, Melanotus castanipes, Austrolebias nigrofasciatus, Sebastes paucispinis, Schoenomyza litorella, Spheniscus magellanicus, Mimagoniates microlepis, Potamonautes margaritarius, Lopidea teton, Epinephelus akaara, Phaonia errans, Chlorodiella cytherea, Leptodactylus latrans, Potamon ibericum, Ligyrocoris sylvestris, Scopula subpunctaria, Scardinius erythrophthalmus, Tropilaelaps mercedesae, Appasus major, Hybomitra sp. BOLD-2016, Cyclotus taivanus, Troides aeacus, Ypthima baldus, Desmia sp. octomaculalisDHJ03, Cyclina sinensis, Tetragonula carbonaria, Lasioglossum villosulum, Protaphis middletonii, Lagopus muta, Stenoma sp. Janzen06, Anopheles pseudopunctipennis, Julus scandinavius, Catostomus bernardini, Helophora reducta, Sarcophaga impatiens, Rachycentron canadum, Aculops lycopersici, Phalloceros harpagos, Ellychnia corrusca, Xestia perquiritata, Banksiola crotchi, Ceratinella brunnea, Dinotrema sp. BOLD: AAX9578, Coenipeta bibitrix, Idaea dimidiata, Testudinella clypeata, Erpobdella punctata, Merodon albifrons, Rana temporaria, Leiobunum ventricosum, Climacia areolaris, Phoebis statira, Pholetesor viminetorum, Erebia medusa, Jikradia olitoria, Leuctra duplicata, Microstomum lineare, Conus chaldaeus, Lasioglossum cressonii, Rhopalias coronatus, Bryotropha hodgesi, Brycinus longipinnis, Anopheles philippinensis, Colias tamerlana, Albula vulpes, Opsaridium microlepis, Corethrella peruviana, Culex environmental sample, Paratendipes sp. BOLD-2016, Astraptes creteus, Rhyssomatus sp. SPN-001, Hydraena gracilis, Placobdella costata, Ephippiger ephippiger, Ozestheria lutraria, Speonomus normandi, Paraliburnia kilmani, Aellopos ceculus, Dynamene edwardsi, Aedes malayensis, Thaumetopoea wilkinsoni, Anagrus atomus, Ligia occidentalis, Montenegrina subcristata, Bathypolypus arcticus, Galathea tanegashimae, Bradybaena circulus, Mongoloniscus sinensis, Austrodiplostomum ostrowskiae, Abrolophus sp. BOLD: AAN6614, Phalaenostola metonalis, Crepidodera aureola, Rhagium inquisitor, Bleptus fasciatus, Cylindroiulus caeruleocinctus, Megaselia sp. 11 LL-2023a, Sericomyrmex amabilis, Symbion pandora, Wulfila saltabundus, Cricetulus longicaudatus, Promachocrinus sp. us ELM-2023, Morellia podagrica, Clinostomum tataxumui, Alycaeus jagori, Acronicta innotata, Phenacoccus aceris, Spilosoma congrua, Nebrioporus baeticus, Sorex tundrensis.

In some aspects, a metabarcoding database of the disclosure further comprises additional unique COI nucleic acid sequences associated with eukaryotes, particularly arthropods and certain genuses/species that can be present in crops, fields, and farms. In some aspects, a database of the disclosure was annotated with 2 to 99 unique COI sequences from the following arthropod genus:

In some aspects, a metabarcoding database of the disclosure further comprises bacterial metabarcoding sequences. A metabarcoding database of the disclosure was constructed comprising at least 5 unique 16S sequences for the following bacterial genus, whereby unique 16S sequences were derived from the United States National Center for Biotechnology Information (NCBI): Streptomyces, Paenibacillus, Pseudomonas, Flavobacterium, Nocardia, Vibrio, Clostridium, Streptococcus, Bacillus, Corynebacterium, Sphingomonas, Microbacterium, Halomonas, Nocardioides, Mycobacterium, Paraburkholderia, Chryseobacterium, Mycolicibacterium, Enterococcus, Rhizobium, Micromonospora, Shewanella, Acinetobacter, Paracoccus, Hymenobacter, Pedobacter, Bifidobacterium, Deinococcus, Amycolatopsis, Actinomadura, Bradyrhizobium, Staphylococcus, Legionella, Mucilaginibacter, Lactobacillus, Pseudonocardia, Lysobacter, Bacteroides, Rhodococcus, Halorubrum, Campylobacter, Actinoplanes, Nonomuraea, Prevotella, Sphingobacterium, Novosphingobium, Burkholderia, Acetobacter, Mesorhizobium, Arthrobacter, Kitasatospora, Pseudoalteromonas, Helicobacter, Sphingobium, Aeromonas, Gordonia, Marinobacter, Methylobacterium, Mycoplasmopsis, Thermococcus, Psychrobacter, Nocardiopsis, Actinomyces, Chitinophaga, Alicyclobacillus, Haloarcula, Spiroplasma, Algoriphagus, Brucella, Agromyces, Bartonell, Brevibacillus, Spirosoma, Yersinia, Brevundimonas, Photobacterium, Pontibacter, Mycoplasma, Virgibacillus, Winogradskyella, Xanthomonas, Fusobacterium, Massilia, Cellulomonas, Marinomonas, Streptosporangium, Porphyromonas, Saccharopolyspora, Brevibacterium, Pantoea, Azospirillum, Natrinema, Devosia, Alteromonas, Serratia, Pectobacterium, Listeria, Leptospira, Cohnella, Leucobacter, Rickettsia, Roseovarius, Klebsiella, Erwinia, Oceanobacillus, Sporomusa, Comamonas, Blautia, Gloeothece, Komagataeibacter, Kribbella, Peptoniphilus, Tenacibaculum, Xenorhabdus, Companilactobacillus, Allomuricauda, Haloferax, Lacticaseibacillus, Brachybacterium, Actinobacillus, Geobacillus, Lysinibacillus, Luteimonas, Lentzea, Sinorhizobium, Microvirga, Caballeronia, Cupriavidus, Treponema, Glycomyces, LimosilactobacillusNeisseria, Leuconostoc, Pandoraea, Polaribacter, Anoxybacillus, Gluconobacter, Kocuria, Metamycoplasma, Aquimarina, Achromobacter, Maribacter, Dyella, Pseudoxanthomonas, Thermoanaerobacter, Lactococcus, Anaerococcus, Sphingopyxis, Sulfitobacter, Methanobacterium, Photorhabdus, Levilactobacillus, Qipengyuania, Microbulbifer, Erythrobacter, Halobacillus, Neobacillus, Peribacillus, Thermus, Stenotrophomonas, Enterobacter, Veillonella, Saccharothrix, Lentilactobacillus, Moraxella, Dyadobacter, Borreliella, Citrobacter, Nitrosomonas, Hydrogenophaga, Exiguobacterium, Dactylosporangium, Sporosarcina, Nesterenkonia, Rheinheimera, Gracilibacillus, Tsukamurella, Herbaspirillum, Metabacillus, Planococcus, Aeromicrobium, Demequina, Undibacterium, Desulfovibrio, Actinokineospora, Weissella, Mesomycoplasma, Dietzia, Microlunatus, Capnocytophaga, Sporolactobacillus, Gluconacetobacter, Ligilactobacillus, Lactiplantibacillus, Pasteurella, Methylorubrum, Agrobacterium, Alistipes, Marinobacterium, Rhodanobacter, Pyrobaculum, Lentibacillus, Phocaeicola, Phenylobacterium, Francisella, Halopseudomonas, Desulfofundulu, Paracidovorax, Rhodovulum, Myroides, Brasilonema, Christiangramia, Caldicellulosiruptor, Halococcus, Bordetella, Halosimplex, Pseudidiomarin, Vagococcus, Prauserella, Aureimonas, Caulobacter, Polynucleobacter, Roseomonas, Thalassotalea, Chlamydia, Pelosinus, Salinicoccus, Brenneria, Geodermatophilus, Arenimonas, Megasphaera, Nonlabens, Methylophaga, Ruegeria, Mycolicibacterm, Nodosilinea, Halomicrobium, Hoylesella, Gemmobacter, Cronobacter, Priestia, Pseudoduganella, Aliidiomarina, Methanobrevibacter, Kineococcus, Methanosarcina, Secundilactobacillus, Mycobacteroides, Selenomonas, Aequorivita, Promicromonospora, Saccharomonospora, Natronococcus, Psychromonas, Salinicola, Colwellia, Cytobacillus, Haemophilus, Pseudoroseomonas, Aneurinibacillus, Parabacteroides, Carnobacterium, Sulfurimonas, Natronorubrum, Chromobacterium, Dickeya, Eubacterium, Curtobacterium, Lachnoclostridium, Corallococcus, Tessaracoccus, Noviherbaspirillum, Hyphomicrobium, Duganella, Phyllobacterium, Halanaerobium, Alkalibacterium, Actinocorallia, Faecalicatena, Actinoallomurus, Clavibacter, Rhodopseudomonas, Mesobacillus, Salegentibacter, Streptacidiphilus, Stutzerimonas, Haloplanus, Acidithiobacillus, Variovorax, Thauera, Cyanothece, Arenibacter, Liquorilactobacillus, Psychroflexus, Microbispora, Pseudodesulfovibrio, Cryobacterium, Pseudarthrobacter, Ornithinimicrobium, Pediococcus, Providencia, Neolewinella, Georgenia, Arcanobacterium, Ferrimonas, Nakamurella, Leifsonia, Tetragenococcus, Planktothrix, Oceanisphaera, Desmonostoc, Olsenella, Geomonas, Williamsia, Marmoricola, Desulfosporosinus, Mesoplasma, Halobellus, Natrialba, Adhaeribacter, Roseibium, Heyndrickxia, Kushneria, Epilithonimonas, Actinopolyspora, Lutibacter, Proteus, Rahnella, Erysipelothrix, Cyclobacterium, Parageobacillus, Tatumella, Macrococcus, Algibacter, Kaistella, Brachyspira, Collinsella, Hyphomonas, Ruminiclostridium, Actinacidiphila, Actinomycetospora, Rubrobacter, Niabella, Archaeoglobus, Glutamicibacter, Catenuloplanes, Schaalia, Mycetocola, Kurthia, Thalassospira, Halorussus, Halobaculum, Arcobacter, Rathayibacter, Bizionia, Granulicella, Desulfomicrobium, Methanoculleus, Ornithinibacillus, Spirochaeta, Sphingorhabdus, Amphibacillus, Thermotoga, Xanthobacter, Catellatospora, Kineosporia, Asaia, Fructilactobacillus, Magnetospirillum, Nitratireductor, Desulfolucanica, Shinella, Sphaerisporangium, Ureibacillus, Sulfurospirillum, Ancylobacter, Agrococcus, Rothia, Thioalkalivibrio, Ramlibacter, Mannheimia, Dysgonomonas, Alkaliphilus, Geobacter, Pseudooceanicola, Jannaschia, Desulforamulus, Streptomonospora, Ralstonia, Mycoplasmoides, Methanolobus, Echinicola, Latilactobacillus, Janibacter, Idiomarina, Bosea, Cylindrospermum, Halomicroarcula, Lederbergia, Chromohalobacter, Kangiella, Lacrimispora, Paeniclostridium, Syntrophomonas, Cellulophaga, Jeotgalibacillus, Methylomonas, Fictibacillus, Dokdonia, Salmonella, Rhodobacter, Aromatoleum, Natronomonas, Nunduva, Raoultella, Blastococcus, Jiangella, Jeotgalicoccus, Aliivibrio, Cellvibrio, Aurantiacibacter, Altererythrobacter, Pelagibacterium, Acidovorax, Methanocaldococcus, Sinomonas, Apilactobacillus, Oceanospirillum, Shouchella, Acetobacterium, Methanothermobacter, Azotobacter, Terrimonas, Thiomicrorhabdus, Alkalihalobacillus, Micrococcus, Terrabacter, Thermoanaerobacterium, Thermomonospora, Solidesulfovibrio, Modestobacter, Salinispora, Halalkalibacter, Isoptericola, Flavisolibacter, Oculatella, Pseudanabaena, Yoonia, Kibdelosporangium, Salipiger, Halogeometricum, Larkinella, Nostoc, Pyrococcus, Leptotrichia, Tissierella, Rufibacter, Acetivibrio, Trichococcus, Paucilactobacillus, Halobacterium, Cutibacterium, Endozoicomonas, Polaromonas, Cryptosporangium, Ruminococcus, Mesonia, Thiomonas, Emticicia, Caldimonas, Natronoarchaeum, Herbidospora, Simplicispira, Afipia, Elizabethkingia, Haladaptatus, Kluyvera, Rhizorhabdus, Delftia, Salinimicrobium, Luteolibacter, Aerococcus, Asticcacaulis, Fervidobacterium, Salinivibrio, Rodentibacter, Novacetimonas, Mediterraneibacter, Meiothermus, Ehrlichia, Aquabacterium, Celeribacter, Paraglaciecola, Falsiroseomonas, Azohydromonas, Shimia, Grimontia, Thermosipho, Roseivivax, Halovivax, Salimicrobium, Leeuwenhoekiella, Ureaplasma, Gemella, Kordia, Rhodoferax, Aliarcobacter, Acholeplasma, Desulfonatronum, Rhodoplanes, Caldanaerobacter, Fructobacillus, Microbacter, Methanocorpusculum, Bhargavaea, Anaerostipes, Desulfitobacterium, Haloarchaeobius, Labrys, Vogesella, Dialister, Thiothrix, Pontibacillus, Allochromatium, Zobellia, Niallia, Salibacterium, Actinophytocola, Cereibacter, Lacinutrix, Slackia, Roseobacter, Ottowia, Avibacterium, Petrotoga, Croceicoccus, Beijerinckia, Sutcliffiella, Kosakonia, Pseudovibrio, Loigolactobacillus, Acidipropionibacterium, Halalkalicoccus, Sulfobacillus, Marichromatium, Halostagnicola, Flammeovirga, Roseivirga, Thioclava, Alcaligenes, Domibacillus, Archangium, Metallosphaera, Intrasporangium, Kaistia, Belliella, Fulvivirga, Chroococcidiopsis, Gelidibacter, Thiocapsa, Cellulosimicrobium, Peterkaempfera, Loktanella, Martelella, Adlercreutzia, Runella, Propionibacterium, Olivibacter, Catenulispora, Methylobacillus, Moritella, Halorhabdus, Thalassovita, Methylophilus, Tumebacillus, Octadecabacter, Phaeobacter, Niastella, Aquamicrobium, Pelomonas, Neptunomonas, Sanguibacter, Tamlana, Moorella, Sinocapsa, Acidiphilium, Chitinibacter, Sediminibacterium, Sutterella, Marinococcus, Caloramator, Kordiimonas, Sorangium, Caldibacillus, Advenella, Tritonibacter, Parapedobacter, Maridesulfovibrio, Alkalibacillus, Belnapia, Timaviella, Thiorhodococcus, Psychrobacillus, Alishewanella, Taibaiella, Gillisia, Evansella, Morganella, Neolyngbya, Nitrincola, Borrelia, Enterocloster, Janthinobacterium, Neorhizobium, Pseudothermotoga, Phycicoccus, Gimesia, Giesbergeria, Asanoa, Desulfoscipio, Formosa, Anaerobranca, Peptostreptococcus, Hyunsoonleella, Gallaecimonas, Azorhizobium, Tsuneonella, Carboxydothermus, Mobiluncus, Rossellomorea, Macrochaete, Siminovitchia, Escherichia, Salicibibacter, Thermomonas, Alcanivorax, Marinitoga, Litoreibacter, Buttiauxella, Sodalis, Citricoccus, Roseburia, Trinickia Hoeflea, Gulosibacter, Desulfuromonas, Thomasclavelia, Halogranum, Pycnacronema, Sneathiella, Anaerocolumna, Planomicrobium, Gallibacterium, Stappia, Flavihumibacter, Aurantimonas, Leisingera, Nitrosopumilus, Aquaspirillum, Halolactibacillus, Alloalcanivorax, Halolamina, Zoogloea, Halopiger, Neoroseomonas, Streptobacillus, Edaphobacter, Aminobacter, Rhodopirellula, Actinosynnema, Trueperella, Teredinibacter, Herminiimonas, Gaetbulibacter, Planomonospora, Mariniflexile, Spongiibacter, Aeropyrum, Methylopila, Chelativorans, Oenococcus, Acidianus, Maricaulis, Nitrosococcus, Sphingomicrobium, Gryllotalpicola, Desulfurococcus, Varibaculum, Paeniglutamicibacter, Atopobium, Thiobacillus, Kutzneria, Lapidilactobacillus, Mycoplana, Pseudoclavibacter, Methanococcoides, Cobetia, Skermanella, Thermoactinomyces, Chitinimonas, Saccharibacillus, Aggregatibacter, Rubritalea, Henriciella, Eggerthella, Blastochloris, Frateuria, Thiomicrospira, Lampropedia, Butyricimonas, Sphingosinicella, Marinospirillum, Haloterrigena, Pleomorphomonas, Palleronia, Kingella, Thiocystis, Hydrogenobacter, Propionispira, Pectinatus, Oceanimonas, Pedococcus, Furfurilactobacillus, Marivita, Pelotomaculum, Pseudaminobacter, Methanofollis, Mogibacterium, Halarchaeum, Halostella, Romboutsia, Roholtiella, Parvularcula, Aliterella, Stenomitos, Methanococcus, Chelatococcus, Limimaricola, Halorientalis, Tabrizicola, Chlorobaculum, Tepidimonas, Methanospirillum, Tenebriella, Reinekea, Ectothiorhodospira, Caldicoprobacter, Pseudorhodobacter, Cystobacter, Olleya, Myxococcus, Plantactinospora, Pollutimonas, Solimonas, Methylocystis, Knoellia, Ruania, Haloferula, Robertmurraya, Salinisphaera, Flaviflexus, Mammaliicoccus, Fibrobacter, Myceligenerans, Flavivirga, Methanohalophilus, Castellaniella, Roseateles, Halorubellus, Phyllonema, Methylotuvimicrobium, Desulfobacter, Acrocarpospora, Odoribacter, Thermaerobacter, Anaerobacillus, Pinocchia, Persephonella, Paraliobacillus, Mixta, Oxalicibacterium, Halorhodospira, Flectobacillus, Thermodesulfovibrio, Actinoalloteichus, Caldivirga, Croceibacterium, Snodgrassella, Thalassolituus, Sphaerochaeta, Gilliamella, Oscillatoria, Blastomonas, Edwardsiella, Spiribacter, Simiduia, Desulfotomaculum, Terriglobus, Actinotignum, Prescottella, Salisediminibacterium, Halanaerobacter, Prosthecobacter, Aquirufa, Inhella, Flexivirga, Enorma, Thermocrinis, Carboxylicivirga, Microcella, Alkalicoccus, Aliiroseovarius, Alteribacter, Thermoclostridium, Aquibacillus, Flaviaesturariibacter, Zunongwangia, Thermobifida, Uliginosibacterium, Fodinibius, Kosmotoga, Acidicapsa, Pseudorhizobium, Collimonas, Bombilactobacillus, Serinicoccus, Sulfurihydrogenibium, Stackebrandtia, Pigmentiphaga, Shimwellia, Actibacterium, Thermodesulfobacterium, Halodesulfovibrio, Zhongshania, Desulfurobacterium, Ensifer, Halobiforma, Diaphorobacter, Hallella, Faecalibacterium, Cedecea, Laspinema, Subtercola, Anaerotignum, Actinotalea, Marivirga, Malaciobacter, Saccharospirillum, Glaciecola, Laceyella, Chlorobium, Paenarthrobacter, Desulfurella, Humibacter, Acidocella, Desulfotignum, Desulfobulbus, Dictyobacter, Dolichospermum, Steroidobacter, Eikenella, Acidaminococcus, Dokdonella, Methanocalculus, Methanothrix, Gordonibacter, Nevskia, Curvibacter, Deferribacter, Kiloniella, Microtetraspora, Salinimonas, Sulfurovum, Desulfosarcina, Methylomicrobium, Entomospira, Empedobacter, Parasphingorhabdus, Sedimentibacter, Reyranella, Sagittula, Terasakiella, Acetomicrobium, Hirschia, Daejeonella, Tautonia, Mojavia, Paraclostridium, Occultella, Novipirellula, Syntrophobacter, Xylanimonas, Zymomonas, Rubellimicrobium, Pilimelia, Novispirillum, Cohaesibacter, Gracilimonas, Halonotius, Fontibacillus, Amylibacter, Limnohabitans, Thermogemmatispora, Mycolicibacillus, Phytoactinopolyspora, Acidihalobacter, Solibacillus, Agarivorans, Fibrella, Wenyingzhuangia, Propioniciclava, Luteococcus, Parafrankia, Thermostaphylospora, Limnobacter, Methylococcus, Terribacillus, Psychrosphaera, Methylobacter, Orenia, Roseospira, Lacibacter, Desulfobotulus, Lancefieldella, Dermabacter, Gemmata, Halovibrio, Christensenella, Phytohabitans, Nisaea, Motilimonas, Draconibacterium, Ciceribacter, Nautilia, Ilyobacter, Planotetraspora, Thalassomonas, Leclercia, Ferruginibacter, Maribellus, Sphaerimonospora, Ideonella, Fusibacter, Elioraea, Chryseomicrobium, Nitrobacter, Aquitalea, Heliorestis, Oerskovia, Periweissella, Hathewaya, Pyrodictiu, Allobacillus, Rhodothermus, Amnibacterium, Butyrivibrio, Chloroflexus, Heliomicrobium, Cloacibacterium, Oxobacter, Halapricum, Dethiosulfovibrio, Chondromyces, Solitalea, Cyanocohniella, Caenispirillum, Syntrophus, Sunxiuginia, Pseudoruegeria, Pseudochrobactrum, Hydrogenophilus, Limnospira, Rubripirellula, Pararhizobium, Luteibacter, Zhihengliuella, Seonamhaeicola, Jonesia, Dictyoglomus, Oligella, Mesotoga, Sulfolobus, Zobellella, Solirubrobacter, Rhizobacter, Leucothrix, Actinopolymorpha, Kineothrix, Coprococcus, Microcoleus, Pelagicoccus, Marinifilum, Marinilactibacillus, Methanogenium, Gracilibacter, Cyanomargarita, Geotalea, Hydrogenovibrio, Marinimicrobium, Granulosicoccus, Stigmatella, Melittangium, Klenkia, Fluviicola, Neorickettsia, Rubinisphaera, Tetrasphaera, Tropicimonas, Alkanindiges, Tepidiphilus, Iningainema, Alloscardovia, Filimonas, Amphritea, Halobacteriovorax, Planifilum, Roseococcus, Jatrophihabitans, Lonsdalea, Brochothrix, Trichotorquatus, Conchiformibius, Glaciimonas, Scytonema, Pelistega, Sinomicrobium, Methanosphaera, Parvibaculum, Alkalispirochaeta, Pseudalkalibacillus, Patulibacter, Polyangium, Nitrosospira, and/or Halobacteroides.

In some aspects, a metabarcoding database of the disclosure further comprises fungi metabarcoding sequences. Among the regions of the ribosomal cistron, the internal transcribed spacer (ITS) region has the highest probability of successful identification for the broadest range of fungi, with the most clearly defined barcode gap between inter- and intraspecific variation. A metabarcoding database of the disclosure was constructed comprising at least 5 unique ITS nucleic acid sequences for the following fungi genuses derived from the United States National Center for Biotechnology Information (NCBI): Cortinarius, Penicillium, Aspergillus, Trichoderma, Colletotrichum, Inocybe, Diaporthe, Phlegmacium, Talaromyces, Alternaria, Fusarium, Amanita, Agaricus, Orbilia, Pseudocercospora, Thaxterogaster, Entoloma, Psathyrella, Russula, Cladosporium, Elsinoe, Lactarius, Cytospora, Ogataea, Phyllosticta, Curvularia, Mucor, Yamadazyma, Calonarius, Pestalotiopsis, Apiospora, Hydnum, Exophiala, Didymella, Hebeloma, Phaeoacremonium, Ophiostoma, Zasmidium, Metschnikowia, Chaetomium, Ramularia, Candida, Marasmius, Cyberlindnera, Lasiodiplodia, Microascus, Teratosphaeria, Derxomyces, Tuber, Hypoxylon, Kazachstania, Scolecobasidium, Pichia, Lactifluus, Cylindrocladiella, Pluteus, Ophiognomonia, Wickerhamomyces, Cercospora, Sticta, Coniochaeta, Distoseptispora, Leucoagaricus, Malbranchea, Suhomyces, Epicoccum, Boletus, Phialocephala, Hygrophorus, Sporothrix, Wickerhamiella, Tomentella, Verrucaria, Diplodia, Calonectria, Otidea, Fomitiporia, Taphrina, Lophiostoma, Tubeufia, Coniella, Neofusicoccum, Cladophialophora, Phanerochaete, Sugiyamaella, Rhizopogon, Starmerella, Leptographium, Mortierella, Candida/Metschnikowiaceae, Neodevriesia, Dothiorella, Metarhizium, Nigrospora, Apiotrichum, Pseudosperma, Tremella, Cyphellophora, Scheffersomyces, Papiliotrema, Blastobotrys, Arthroderma, Kockovaella, Sarocladium, Emericellopsis, Sporobolomyces, Bipolaris, Claviceps, Hygrocybe, Ganoderma, Dothiora, Cora, Acaulospora, Dentiscutata, Neopestalotiopsis, Stemphylium, Phylloporus, Pezicula, Hypomyces, Coprinopsis, Gymnopus, Kondoa, Saccharata, Erysiphe, Dioszegia, Naganishia, Lepiota, Ceratocystis, Humicola, Toxicocladosporium, Neocucurbitaria, Trichophyton, Plagiostoma, Xylaria, Cyanosporus, Stagonosporopsis, Saccharomycopsis, Clonostachys, Debaryomyces, Absidia, Vishniacozyma, Harknessia, Mycena, Malassezia, Melanoleuca, Saturnispora, Hodophilus, Geastrum, Gaeumannomyces, Hymenoscyphus, Myrmecridium, Ramaria, Hymenochaete, Lipomyces, Microdochium, Roussoella, Xylodon, Suillus, Cutaneotrichosporon, Seiridium, Septoriella, Inosperma, Septoria, Tricholoma, Cadophora, Nakazawaea, Yarrowia, Thyronectria, Dinemasporium, Funneliformis, Auricularia, Cephalotrichum, Beauveria, Bulleribasidium, Spathaspora, Daldinia, Nigrograna, Helminthosporium, Periconia, Stagonospora, Hanseniaspora, Chrysosporium, Rhodotorula, Symmetrospora, Muyocopron, Kodamaea, Tolypocladium, Knufia, Anthracocystis, Nemania, Paramyrothecium, Ophiocordyceps, Rhodosporidiobolus, Castanediella, Phaeosphaeria, Crepidotus, Teunomyces, Perenniporia, Clitopilus, Ascosphaera, Venturia, Keratinophyton, Cladobotryum, Kwoniella, Bambusicola, Neosetophoma, Parafenestella, Podospora, Rachicladosporium, Nannizziopsis, Rasamsonia, Lecanicillium, Trichomerium, Capronia, Readeriella, Kurtzmaniella, Paraphaeosphaeria, Colacogloea, Rhodocybe, Teichospora, Nothophoma, Terfezia, Filobasidium, Nannizzia, Neoascochyta, Paramycosphaerella, Lachancea, Diatrypella, Genea, Cyathus, Coleophoma, Leucosporidium, Barnettozyma, Hyphopichia, Aureobasidium, Camarosporidiella, Cystobasidium, Cantharellus, Neohelicosporium, Trichosporon, Cosmospora, Phlebiopsis, Glutinoglossum, Laccaria, Heterotruncatella, Sphaerulina, Simplicillium, Plectosphaerella, Verticillium, Keissleriella, Clavaria, Dichotomopilus, Rhinocladiella, Lobariella, Chlorociboria, Ceramothyrium, Alfaria, Phallus, Exserohilum, Linnemannia, Collybiopsis, Huntiella, Clavulina, Cordyceps, Raffaelea, Melanconiella, Setophoma, Arthrobotrys, Caloplaca, Graphium, Thelonectria, Paraconiothyrium, Gliocladiopsis, Phaeosphaeriopsis, Hannaella, Gnomoniopsis, Entyloma, Elaphomyces, Pleurotheciella, Leptosillia, Puccinia, Aureonarius, Lopadostoma, Dictyocheirospora, Tetraploa, Stephanospora, Mollisia, Saccharomyces, Mrakia, Peziza, Diversispora, Thyrostroma, Arthrinium, Codinaea, Schwanniomyces, Fusicolla, Paraphoma, Sporidesmium, Sporocadus, Cladonia, Jorgewrightia, Backusella, Lindgomyces, Parastagonospora, Ambrosiozyma, Discosia, Spiromastigoides, Chrysozyma, Dictyosporium, Seimatosporium, Acremonium, Helicoma, Peniophorella, Cordana, Cryptosporella, Gliocephalotrichum, Botryosphaeria, Torula, Oidiodendron, Fuscoporia, Harzia, Paecilomyces, Endoraecium, Tortispora, Phaeotremella, Umbelopsis, Calophoma, Helvella, Basidiodendron, Rhynchogastrema, Parasola, Fenestella, Meyerozyma, Pseudocamarosporium, Hamigera, Geosmithia, Grosmannia, Wojnowiciella, Hydnellum, Diutina, Leptosphaeria, Parasarocladium, Zanclospora, Pseudophialophora, Phialophora, Helicosporium, Mariannaea, Gyromitra, Ilyonectria, Memnoniella, Subulicystidium, Montagnula, Plenodomus, Priceomyces, Phialemonium, Phellodon, Calogaya, Massaria, Vanrija, Dendrostoma, Neurospora, Lecanora, Chaetopsina, Nakaseomyces, Wallemia, Melanconis, Torulaspora, Thyridium, Dacrymyces, Gymnopilus, Pseudolachnella, Pyrenochaetopsis, Kellermania, Hermatomyces, Dasyspora, Phaeoisaria, Conocybe, Aplosporella, Pseudocyphellaria, Solicoccozyma, Myrtapenidiella, Phellinus, Neopyrenochaeta, Fulvifomes, Microsporomyces, Diatrype, Pholiota, Tetrapisispora, Conioscypha, Gyroporus, Cryptocoryneum, Caliciopsis, Pseudoophiobolus, Celoporthe, Escovopsis, Volvanarius, Entomocorticium, Ascochyta, Phyllozyma, Kluyveromyces, Geoglossum, Butyriboletus, Paragaeumannomyces, Neodeightonia, Gymnascella, Polyscytalum, Cylindrium, Pyricularia, Jattaea, Piskurozyma, Komagataella, Ramalina, Myxozyma, Collophorina, Resinicium, Microglossum, Pachyphlodes, Neocamarosporium, Lyomyces, Lecanosticta, Bensingtonia, Favolaschia, Xenopenidiella, Preussia, Sclerostagonospora, Murispora, Paraisaria, Nectria, Scleroderma, Dactylonectria, Quambalaria, Ruinenia, Microbotryum, Gomphus, Leohumicola, Parmelia, Peroneutypa, Monochaetia, Tubakia, Akanthomyces, Neohelicomyces, Zygoascus, Racocetra, Stegonsporium, Porodaedalea, Achroiostachys, Boeremia, Kuraishia, Fomitopsis, Trichomonascus, Tricharina, Allophoma, Pseudozyma, Favolus, Schizothyrium, Tarzetta, Phyllachora, Cladorrhinum, Botrytis, Neofabraea, Beltraniella, Staphylotrichum, Ambrosiella, Sulzbacheromyces, Crinipellis, Paracremonium, Arthrographis, Ceriporia, Peniophora, Phylloporia, Chaetocapnodium, Strelitziana, Bacidia, Goffeauzyma, Subramaniula, Sidera, Gibbosporina, Darksidea, Fonsecaea, Auritella, Comoclathris, Zymoseptoria, Octaviania, Megacollybia, Zygosaccharomyces, Pseudocosmospora, Minimelanolocus, Leptospora, Sistotremastrum, Moniliella, Acaulium, Phaeoclavulina, Corynespora, Pilidium, Tilletia, Botryotrichum, Podosphaera, Codinaeella, Amphisphaeria, Parachaetomium, Ovatospora, Trichocladium, Calocybella, Ramichloridium, Arxotrichum, Lichtheimia, Hohenbuehelia, Pyrenophora, Stachybotrys, Verruconis, Lentomitella, Neoceratosperma, Rhizopus, Neoroussoella, Efibula, Pseudofusicoccum, Valsaria, Polyporus, Pseudoteratosphaeria, Pseudosydowia, Tubaria, Morchella, Cystofilobasidium, Rhizoplaca, Meliola, Phaeosaccardinula, Xenodidymella, Parathyridaria, Paranannizziopsis, Myrothecium, Striatibotrys, Graphilbum, Golovinomyces, Endoconidiophora, Hypoderma, Thermothelomyces, Acrophialophora, Dendryphiella, Paracamarosporium, Blastomyces, Magnaporthiopsis, Arachnomyces, Tremateia, Bradymyces, Sarcogyne, Neodidymelliopsis, Atractospora, Podila, Penidiella, Remotididymella, Striaticonidium, Cercophora, Apophysomyces, Neoprotoparmelia, Halobyssothecium, Paracylindrocarpon, Disculoides, Lectera, Rhytidhysteron, Metapochonia, Pseudopestalotiopsis, Brettanomyces, Pseudochaetosphaeronema, Coryneum, Polyozellus, Phaeococcomyces, Gongronella, Triangularia, Tylopilus, Psilocybe, Auxarthronopsis, Knoxdaviesia, Phyllactinia, Scytinostroma, Monilochaetes, Starmera, Pithoascus, Zopfiella, Hypotrachyna, Macrolepiota, Monascus, Pseudeurotium, Monocillium, Eremothecium, Bullera, Chroogomphus, Metulocladosporiella, Glomus, Heitmania, Pseudopyricularia, Scedosporium, Constantinomyces, Pyrenula, Baudoinia, Sclerococcum, Gymnoascus, Hydnotrya, Buckleyzyma, Hydnophlebia, Mallocybe, Calocybe, Neophaeomoniella, Stanjemonium, Trametes, Sakaguchia, Parateratosphaeria, Neonectria, Triadelphia, Pseudohyphozyma, Gamszarea, Redeckera, Sphaeropsis, Emergomyces, Erythrobasidium, Hirsutella, Sarcodon, Barbatosphaeria, Xerocomus, Kirschsteiniothelia, Entomortierella, Vermiculariopsiella, Lasionectria, Robillarda, Scopulariopsis, Ceriporiopsis, Abrothallus, Mycosphaerella, Chlorophyllum, Acrocalymma, Atrocalyx, Wardomyces, Coccomyces, Phaeophleospora, Farysia, Ophiobolus, Alanphillipsia, Teratoramularia, Phlogicylindrium, Juncaceicola, Westerdykella, and/or Buwchfawromyces.

### Embodiments

EMBODIMENT 1. A process for screening an agricultural unit for a health risk to the agricultural unit, comprising: generating a plurality of nucleic acid sequencing reads from one or more sample(s) from the agricultural unit; screening the plurality of nucleic acid sequencing reads derived from the sample(s) gathered from the agricultural unit for a pattern that identifies the health risk to the agricultural unit, whereby the screening is performed by a computer program model trained to identify the pattern from a plurality of features, whereby one or more features comprises the abundance of the plurality of taxa found in the agricultural unit and outputting the risk score that is predictive of the health risk to the agricultural unit based on the screening.
EMBODIMENT 2. The process of Embodiment 1, whereby the health risk to the agricultural unit is a risk of crop failure or damage.
EMBODIMENT 3. The process of Embodiment 1, wherein the health risk to the agricultural unit is a risk of a pest infestation.
EMBODIMENT 4. The process of Embodiment 1, wherein the health risk to the agricultural unit is a risk of a depletion of organic matter.
EMBODIMENT 5. The process of Embodiment 1, wherein the health risk to the agricultural unit is a risk of a soil mineralization.
EMBODIMENT 6. The process of Embodiment 1, wherein the health risk to the agricultural unit is a risk of depletion of beneficial microbiota from the soil or from within the crop.
EMBODIMENT 7. The process of Embodiment 1, wherein the plurality of nucleic acid sequences extracted from the agricultural unit comprises:
   a plurality of nucleic acid sequences extracted from at least one arthropod;
   a plurality of nucleic acid sequences extracted from at least one nematode;
   a plurality of nucleic acid sequences extracted from at least one bacterium;
   a plurality of nucleic acid sequences extracted from at least one fungus/mold/yeast;
   a plurality of nucleic acid sequences extracted from at least one protozoa;
   a plurality of nucleic acid sequences extracted from at least one archaea; and/or
   a plurality of nucleic acid sequences extracted from at least one virus.
EMBODIMENT 8. The process of Embodiment 7, wherein the computer program model identifies a taxa in the agricultural unit based on a plurality of chromosomal or mitochondrial metabarcode nucleic acid sequences selected from the group comprising Cytochrome Oxidase I, Cytochrome b, 16S rRNA, ITS in the plurality of nucleic acid sequences extracted from the at least one arthropod.
EMBODIMENT 9. The process of Embodiment 7, wherein the computer program model identifies a taxa in the agricultural unit based on a plurality of chromosomal or mitochondrial metabarcode nucleic acid sequences selected from the group comprising Cytochrome Oxidase I, Cytochrome b, 18S small subunit rRNA, and 28S large subunit rRNA in the plurality of nucleic acid sequences extracted from the at least one nematode.
EMBODIMENT 10. The process of Embodiment 7, wherein the computer program model identifies a taxa in the agricultural unit based on a plurality of chromosomal or mitochondrial metabarcode nucleic acid sequences selected from the group comprising 16S rRNA, ITS in the plurality of nucleic acid sequences extracted from the at least one bacterium or archaea.
EMBODIMENT 11. The process of Embodiment 7, wherein the computer program model identifies a taxa in the agricultural unit based on a plurality of chromosomal or mitochondrial metabarcode nucleic acid sequences selected from the group comprising 16S rRNA and ITS in the plurality of nucleic acid sequences extracted from the at least one fungus.
EMBODIMENT 12. The process of Embodiment 7, wherein the computer program model identifies a taxa in the agricultural unit based on a plurality of chromosomal or mitochondrial metabarcode nucleic acid sequences selected from the group comprising 16S rRNA, 18S rRNA in the plurality of nucleic acid sequences extracted from the at least one protozoa.
EMBODIMENT 13. The process of Embodiment 7, wherein the computer program model identifies a virus in the agricultural unit based on targeted amplicon sequencing assay.
EMBODIMENT 14. The process of any one of Embodiments 8-13, whereby the process identifies larvae, nymph, eggs, and mature taxa.
EMBODIMENT 15. The process of Embodiment 7, wherein the processing of the sample of the agricultural unit comprises isolating a plurality of nucleic acids from the sample and processing the plurality of nucleic acids for sequencing.
EMBODIMENT 16. The process of Embodiment 15, wherein the extracted nucleic acids are enriched via primer-based target amplification.
EMBODIMENT 17. The process of Embodiment 16, wherein the set of primers for primer-based target amplification comprises markers for insecticide resistance, antibiotic, and/or anti-fungal resistance marker.
EMBODIMENT 18. The process of Embodiment 15, wherein the extracted nucleic acids are enriched via an amplification method selected from the group comprising of: PCR, RPA, NASBA, LAMP, TMA, MDA, RCA, and HAD amplification methods.
EMBODIMENT 19. The process of Embodiment 15, wherein the extracted nucleic acids are enriched via hybridization-based target amplification using a panel of capture probes selected from the group comprising of: DNA capture probes, ssDNA capture probes, and RNA capture probes.
EMBODIMENT 20. The process of Embodiment 15, wherein the hybridization-based target amplification is a solid-phase target capture assay, a bead-capture assay, or a liquid-phase capture assay.
EMBODIMENT 21. The process of Embodiment 15, wherein the extracted nucleic acids are enriched via aptamer-based capture assay or molecular inversion probes based capture assay.
EMBODIMENT 22. The process of Embodiment 15, wherein the sequencing comprises Sanger sequencing.
EMBODIMENT 23. The process of Embodiment 15, wherein the sequencing comprises next-generation selected from the group consisting of: sequencing-by-synthesis, semiconductor sequencing, avidity sequencing, single-read flow-based sequencing, and sequencing-by-binding.
EMBODIMENT 24. The process of Embodiment 15, wherein the sequencing comprises third-generation sequencing such as single molecule real-time sequencing (SMRT by Pacific Biosciences) or nanopore sequencing (Oxford Nanopore Technology).
EMBODIMENT 25. The process of Embodiment 1, wherein the screening of the plurality of features comprises screening the plurality of nucleic acid sequences extracted from the agricultural unit and forming a first list of raw sequence reads.
EMBODIMENT 26. The process of Embodiment 25, wherein the screening comprises forming a second list of clusters of conserved metasequences present in the first list of raw sequence reads.
EMBODIMENT 27. The process of Embodiment 26, wherein the computer program model produces a score from the second list of clusters of conserved metasequences.
EMBODIMENT 28. The process of Embodiment 1, wherein the computer program product is trained to identify the pattern from at least one million features.
EMBODIMENT 29. The process of Embodiment 1, wherein the computer program product is trained to identify the predictive pattern with a sensitivity of 90%. 2
EMBODIMENT 30. The process of Embodiment 1, wherein the computer program product is trained to identify the predictive pattern with a specificity of 90%.
EMBODIMENT 31. A system for screening an agricultural unit for a taxa, the system comprising:
   a receiving module for receiving a plurality of primary nucleic acid sequences extracted from at least one sample collected from the agricultural unit;
   a database comprising a plurality of nucleic acid sequences uniquely associated with a taxa, the database comprising:
      a plurality of nucleic acid sequences extracted from at least one arthropod;
      a plurality of nucleic acid sequences extracted from at least one nematode;
      a plurality of nucleic acid sequences extracted from at least one bacterium;
      a plurality of nucleic acid sequences extracted from at least one fungus/mold/yeast;
      a plurality of nucleic acid sequences extracted from at least one protozoa; and/or
      a plurality of nucleic acid sequences extracted from at least one archaea;
   a screening module for comparing the plurality of primary nucleic acid sequences received by the receiving module against the plurality of nucleic acid sequences uniquely associated with the taxa in the database; and 19
   an output module for outputting a list of taxa present in the at least one sample from the agricultural unit.
EMBODIMENT 32. A system for calculating a health risk score to an agricultural unit, the system comprising:
   a receiving module for receiving a plurality of taxa of species outputted by the screening system in the claim 31, derived from the nucleic acid sequences extracted from at least one sample collected from the agricultural unit, the plurality of nucleic acid sequences selected from the group consisting of:
      a plurality of nucleic acid sequences extracted from at least one arthropod;
      a plurality of nucleic acid sequences extracted from at least one nematode;
      a plurality of nucleic acid sequences extracted from at least one bacterium;
      a plurality of nucleic acid sequences extracted from at least one fungus/mold/yeast;
      a plurality of nucleic acid sequences extracted from at least one protozoa; and/or 2
      a plurality of nucleic acid sequences extracted from at least one archaea;
   a screening module for comparing the qualitative and quantitative data of the taxa and their respective abundance received by the receiving module with a machine learning model trained to provide a health risk score to the agricultural unit; and
   an output module for outputting the compounded health risk score to the agricultural unit based on the plurality of the taxa and their respective abundance. 8
EMBODIMENT 33. The system of Embodiment 31, wherein the screening module further creates a secondary list of clusters of sequences from the primary nucleic acid sequences based on conserved metasequences listed on the database to be uniquely associated with a taxa.
EMBODIMENT 34. The system of Embodiment 32, wherein the screening module further creates a list of individual risk scores for the agricultural unit from the qualitative and 13 quantitative presence of taxa in different sample types received by the receiving module based on other data about the agricultural unit such as crop type, location, etc.
EMBODIMENT 35. The system of Embodiment 34, wherein the computer program model produces a compounded risk score from the secondary list of individual risk scores.
EMBODIMENT 36. The system of any one of Embodiments 31-32, wherein the plurality of primary nucleic acid sequences extracted from the agricultural unit comprises at least two of, at least three of, at least four of, at least five of, or all six of:
   a plurality of nucleic acid sequences extracted from at least one arthropod;
   a plurality of nucleic acid sequences extracted from at least one nematode;
   a plurality of nucleic acid sequences extracted from at least one bacterium;
   a plurality of nucleic acid sequences extracted from at least one fungus/mold/yeast;
   a plurality of nucleic acid sequences extracted from at least one protozoa; and
   a plurality of nucleic acid sequences extracted from at least one archaea.
EMBODIMENT 37. The system of any one of Embodiments 31-32, wherein the screening module identifies a taxa in the agricultural unit based on a plurality of chromosomal or mitochondrial metabarcode nucleic acid sequences selected from the group comprising Cytochrome Oxidase I, Cytochrome b, 16S rRNA, ITS, in the plurality of nucleic acid sequences extracted from the at least one arthropod, nematode, bacterium, and/or archaea.
EMBODIMENT 38. The system of any one of Embodiments 31-32, wherein the screening module identifies a taxa in the agricultural unit based on a 16S rRNA nucleic acid sequence and/or an internal transcribed spacer (ITS) sequence in the plurality of nucleic acid sequences extracted from the at least one fungus.
EMBODIMENT 39. The system of any one of Embodiments 31-32, wherein the screening module identifies a taxa in the agricultural unit based on a 18S rRNA nucleic acid sequence in the plurality of nucleic acid sequences extracted from the at least one protozoa.
EMBODIMENT 40. The system of any one of Embodiments 31-39, whereby the screening module identifies larvae, nymph, eggs, and mature taxa based on the plurality of nucleic acid sequences uniquely associated with a taxa.
EMBODIMENT 41. The system of Embodiment 32, wherein the output module further provides a list of all taxa identified by the screening module.
EMBODIMENT 42. The system of Embodiment 32, wherein the database comprises at least 40,000 nucleic acid sequences uniquely associated with greater than 7,100 genera of bacteria and fungi.
EMBODIMENT 43. The system of Embodiment 32, wherein the database comprises at least 3.4 million nucleic acid sequences uniquely associated with greater than of 510,000 genera of arthropods.
EMBODIMENT 44. The system of Embodiment 32, wherein the database comprises at least 100,000 nucleic acid sequences uniquely associated with greater than of 1,000 genera of nematodes.
EMBODIMENT 45. The system of Embodiment 32, wherein the database comprises at least 100,000 nucleic acid sequences uniquely associated with greater than of 1,000 genera of protozoa.
EMBODIMENT 46. The system of Embodiment 32, wherein the database comprises at least 100,000 nucleic acid sequences uniquely associated with greater than of 1,000 genera of archaea.
EMBODIMENT 47. The system of Embodiment 32, whereby the health risk score provides a measure of a risk of crop failure or damage.
EMBODIMENT 48. The system of Embodiment 32, whereby the health risk score provides a measure of a risk of a pest infestation.
EMBODIMENT 49. The system of Embodiment 32, whereby the health risk score provides a measure of a risk of a depletion of organic matter.
EMBODIMENT 50. The system of Embodiment 32, whereby the health risk score provides a measure of a risk of a soil mineralization.
EMBODIMENT 51. The system of Embodiment 32, whereby the health risk score provides a measure of a risk of depletion of beneficial microbiota from the soil or from within the crop.
EMBODIMENT 52. The system of Embodiment 32, whereby the health risk score provides a measure of a risk of infection of water-borne pathogens by measuring the abundance of water-borne pathogens in the irrigation water or farm run-off water.

### 12 EXAMPLES

The present technology is not to be limited in terms of the particular implementations described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular implementations only, and is not intended to be limiting.

### Example 1: Case Study of Application to Different Citrus Orchards

In this application, we describe a study of the system and processes of the disclosure. In this study, yellow sticky traps (insect) were collected from 14 different citrus orchards that were spread across an area of 2500 sq. miles in the central valley in California. These insect traps were then shipped at room temperature and processed in our laboratory in Pleasanton, CA. Two different specimens of Asian Citrus Psyllid (ACP, Taxonomic name Diaphorina citri) were also processed alongside these insect trap samples as positive controls. 2

In this embodiment, the model leveraged the power of highly accurate targeted short-read sequencing to differentiate the amplicon sequence variations of a few universal barcodes present in the mitochondrial DNA of various species to detect a very broad range of insect pests, concurrently in a large number of samples. This method is highly advantageous compared to other conventional methods that rely on the differences in the physical structure and shape of insects and may not have the species-level resolution. A plurality of sequencing rads were 8 obtained. As demonstrated in the results below, the model further provided high sensitivity for detecting fragments of the insects from their different metamorphic stages. This is particularly desirable for agricultural purposes. This test can have a number of practical applications to the agriculture industry:

### I. Experimental Methods

Each of the insect traps was first treated and washed with roughly 5 ml of 70% ethanol to collect all the insect material collected on the traps into a trough. Then, the insect matter was homogenized thoroughly into a paste and about 1g was sub-sampled for cell lysis and DNA extraction. The same process was followed for the pure ACP specimens as well. The samples were then subjected to cell lysis by incubating at 56C overnight in Lysing matrix A (from MP Biomedicals). Then DNA was extracted and purified from the respective samples using a modified version of the NucleoSpin Tissue kit (from Machery Nagel). The elution volume was limited to produce a concentrated amount of purified extracted DNA, which was quantified using Qubit and subsequently used for sequencing library preparation. Each sample was processed as two replicates to ensure that the reported taxa is reproducible.

Universal cytochrome oxidase I (abbreviated as COI) metabarcode markers were selectively amplified from the genomic DNA of each of the samples. The amplicons from each of the samples were cleaned up using a bead-based purification method, attached to unique dual sample-level barcodes, followed by Illumina sequencing adapters and subsequently sequenced on MiSeq sequencer using the 2×250 bp v2 nano kit for approximately 27 hours. Multiple run-level controls were included in this sequencing run as well. The FASTQ data from the sequencer was then analyzed using our proprietary bioinformatic analysis software and a specially-curated database to provide sample-level identification results. The results were then manually verified to create a sample-level taxonomical ID report. 2

### II. Results & Discussion

Within the 14 insect trap samples, the Evolve Pest-Seq™ test was able to detect & Identify 53 unique genera of insects, 33 unique species, and to resolve 55% of samples taxonomically to species-level resolution based on the COI metabarcode markers.

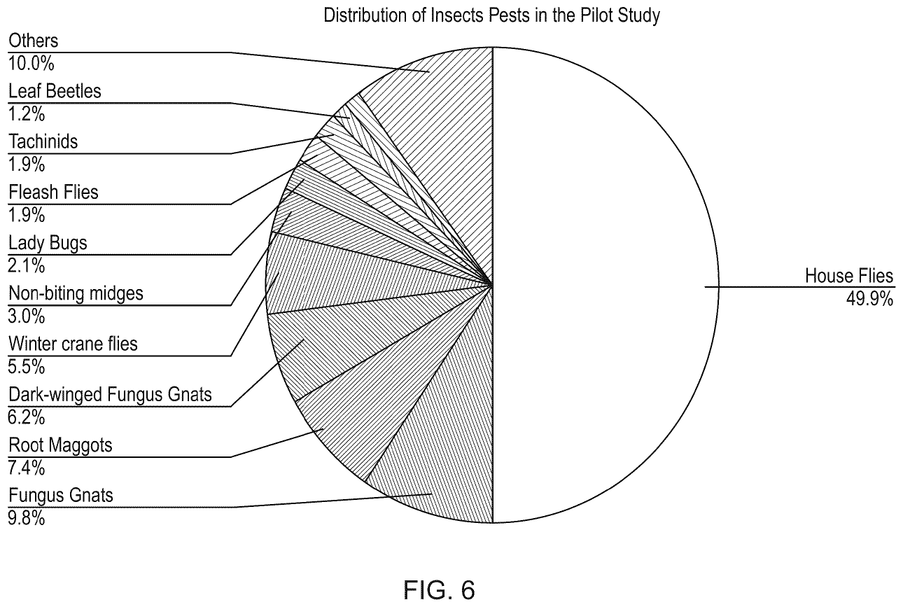

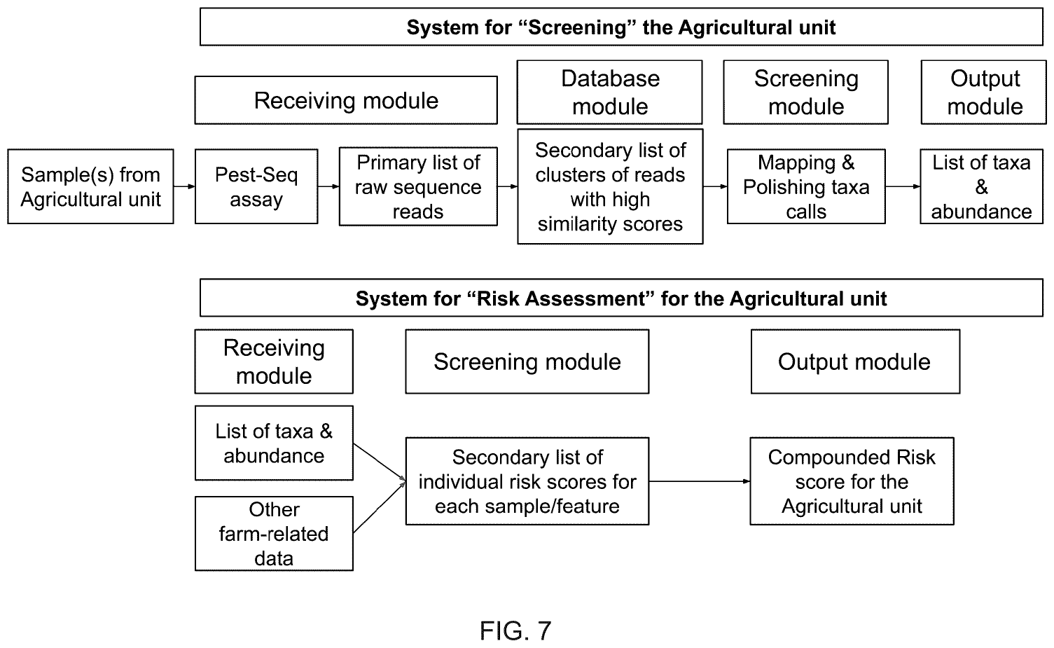

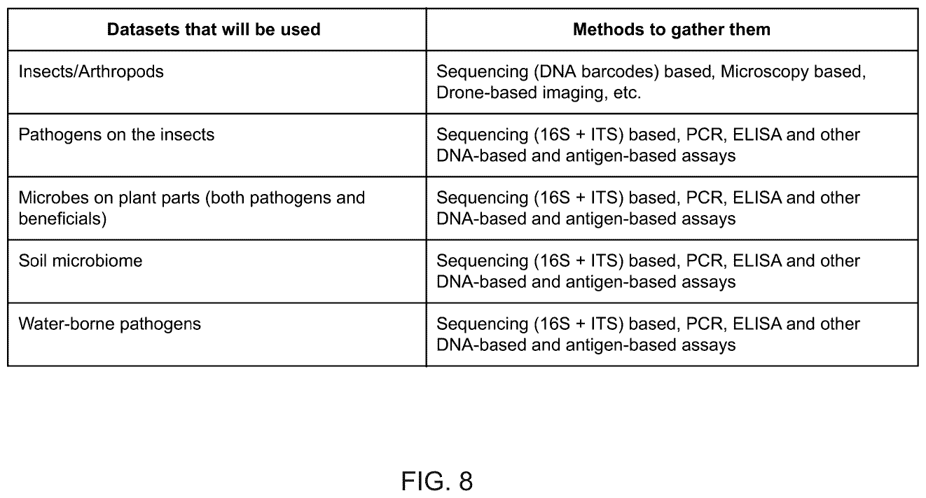

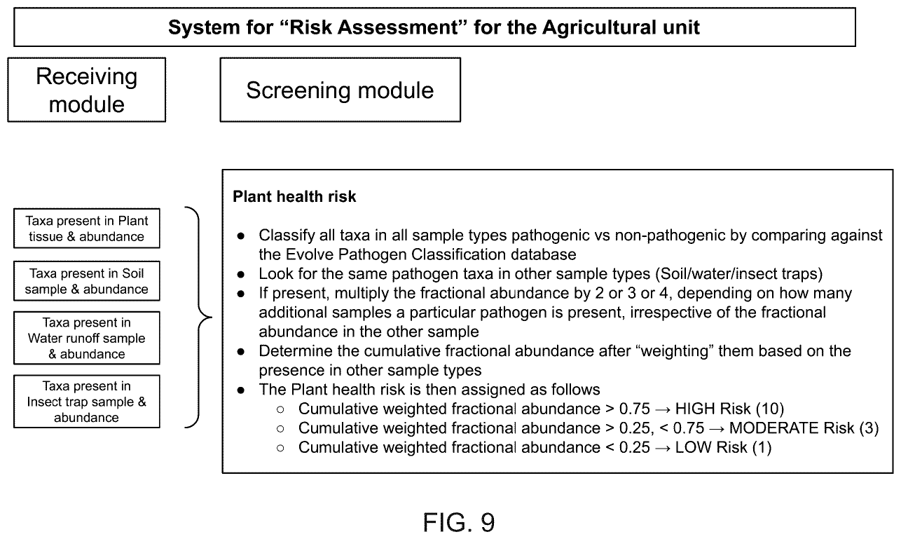

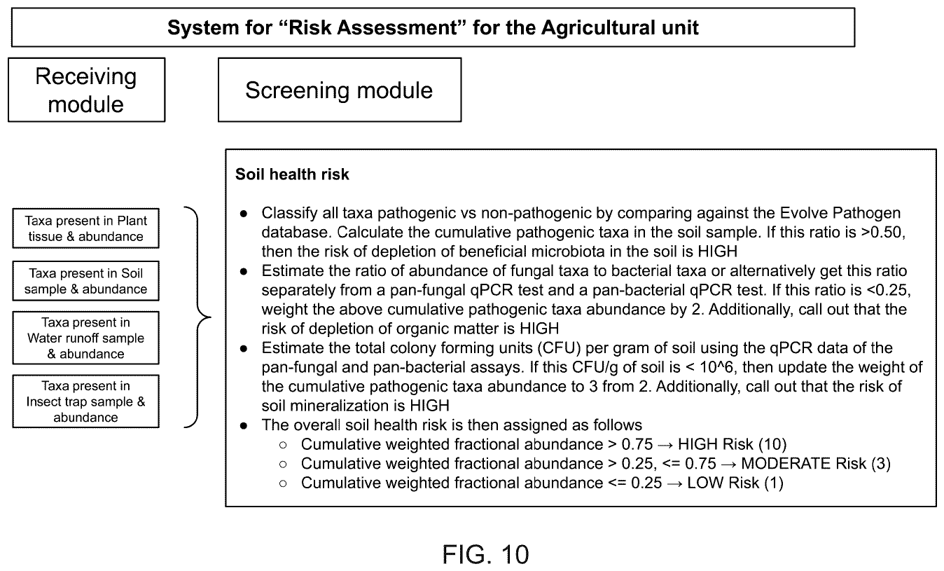

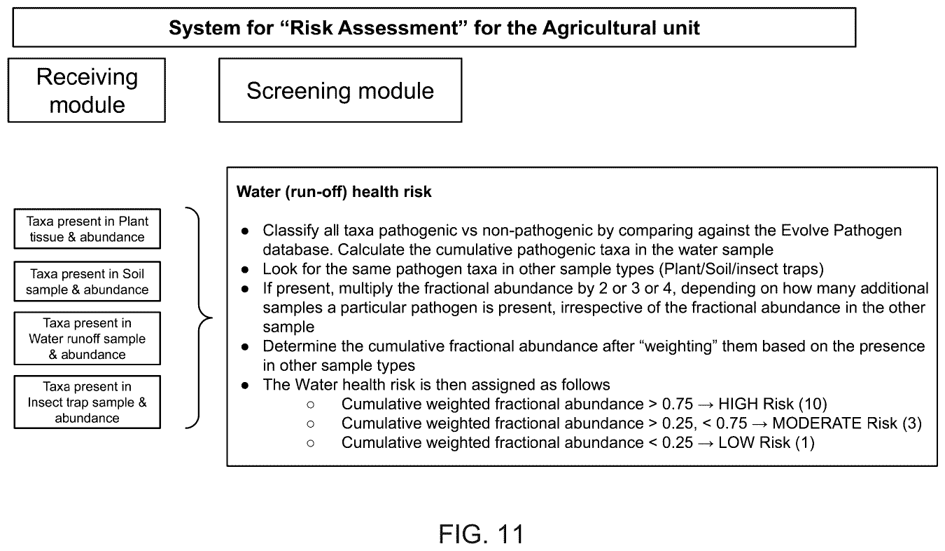

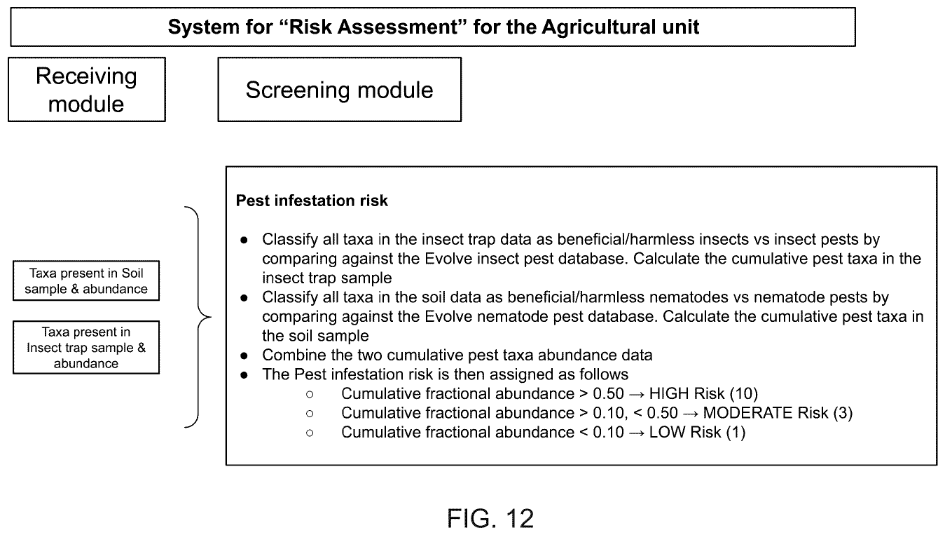

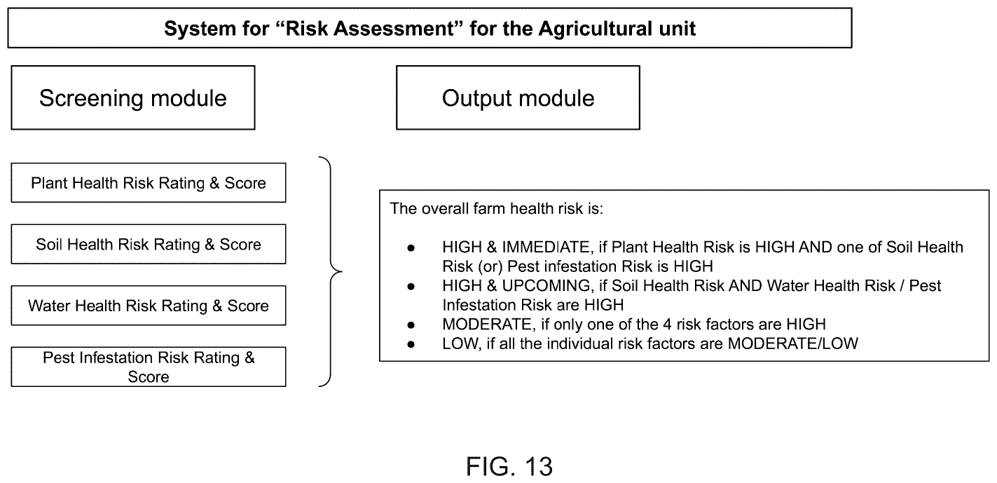

Because the Evolve Pest-Seq™ assay targets the cytochrome oxidase subunit I (COI) region, which is well characterized and has a very large reference database, the assay was able to identify with great resolution a large number of species and genus. A high-level distribution of the different insect families identified in this study is shown in FIG. 6. While almost 50% of the insects captured in the insect traps used in this pilot study belonged to the family Muscidae (House flies), the assay also identified an unexpected number of insects belonging to very different families.

Out of the 14 different citrus orchards tested, some were reported to be afflicted by an unquantified degree of fungal rot diseases and discoloration of fruits. Interestingly, nearly 25% of common insects (Fungus gnats, Black-winged fungus gnats and Root maggots) could cause the observed rot and discoloration. A distribution of the Taxonomical ID of pests found in orchard 1 is described in Table 1.

TABLE 3

| | Taxonomical ID of Pests | % of DNA in Insect |
|---|---|---|
| Sample ID | Found | Trap |
| Orchard 1 | Delia platura | 44.9 |
| Orchard 1 | Muscina levida | 31.3 |
| Orchard 1 | Scatopsciara atomaria | 7.5 |
| Orchard 1 | Helicobia rapax | 5.1 |
| Orchard 1 | Coenosia Sp. | 4.2 |
| Orchard 1 | Chrysoperia Sp. | 2.1 |
| Orchard 1 | Apis Sp. | 1.7 |
| Orchard 1 | Phasia Sp. | 1.1 |
| Orchard 1 | Ceratagallia omani | 1.1 |
| Orchard 1 | Pegomya betae | 0.3 |
| Orchard 1 | Macrosteles Sp. | 0.2 |
| Orchard 1 | Lasioglossum Sp. | 0.2 |
| Orchard 1 | Musca domestica | 0.1 |
| Orchard 1 | Lonchaea cristula | 0.1 |

The two positive controls (ACP) ran along with the insect trap samples were identified correctly as Diaphorina citri. The only other species present in one of the positive controls is the fungus Penicillium citrinum, which is a common fungal species present sometimes in moldy citrus fruits. It is quite possible that the control ACP insect specimen might have contacted this mold and it was present on the body of the insect.

### Example 2: Case Study of Application of system for triangulating causal agents for a Plant disease

In this application, the processes and system of the disclosure were used to investigate the causal pathogens for a “rot” disease in lemon ranches. Three diseased lemon trees (from ranches -“5” and “10”) and two healthy lemon trees were considered for this study. Lemon leaf and bark tissue samples from these two groups of trees were homogenized, subjected to lysis and nucleic acid extraction using Zymo Plant/seed miniprep DNA kit. The microbial diversity present in the DNA extracted from these 5 samples was identified using the processes and system of the disclosure. In parallel, insect traps were collected from the two ranches “5” and “10” and DNA was extracted from the homogenized insect soup for each of the respective ranches. The microbial diversity present in the insect gut contents was evaluated from the DNA extracted using the processes and system of the disclosure.

The tables below provide the results of the assay:

TABLE 4
Bacterial Results from Samples of Diseased
Lemon Tissue from Ranch 10A

| | | | |
|---|---|---|---|
| | % of bacterial | Plant | Reads |
| Name of bacteria | reads | Pathogen | count |

| | | |
|---|---|---|
| Sodalinema orleanskyi | 28.2 | No |
| Hymenobacter spp. | 21.4 | No |
| Sphingomonas spp. | 16.9 | No |
| Devosia albogilva | 8.9 | No |
| Methylobacterium spp. | 7.5 | No |
| Massilia aurea | 6.1 | No |
| Klenkia marina | 4.3 | No |
| Variovoraxgossypii | 3.0 | No |
| Kineococcusrubinsiae | 2.3 | No |
| Trichotorquatus coquimbo | 1.3 | No |

TABLE 5
Fungi Results from Samples of Diseased
Lemon Tissue from Ranch 10A

| | % of fungal | Plant | Reads |
|---|---|---|---|
| Name of fungi | reads | Pathogen | count |
| Aureobasidium pullulans | 70.3 | Opportunistic | Very high |
| Cryptococcus magnus | 13.6 | No | |
| Alternaria spp. | 7.3 | Yes | High |
| Dioszegia butyracea | 4.0 | No | |
| Erythrobasidium yunnanense | 3.2 | No | |
| Neodidymelliopsis ranunculi | 1.7 | No | |

TABLE 6
Bacterial Results from Samples of Diseased
Lemon Tissue from Ranch 10B

| | | | |
|---|---|---|---|
| | % of bacterial | Plant | Reads |
| Name of bacteria | reads | Pathogen | count |

| | | |
|---|---|---|
| Methylobacterium spp. | 33.9 | No |
| Sphingomonas spp. | 28.0 | No |
| Massilia spp. | 14.0 | No |
| Roseomonas vinacea | 10.4 | No |
| Frigoribacterium endophyticum | 8.2 | No |
| Hymenobacter frigidus | 3.2 | No |
| Novosphingobium olei | 2.2 | No |

TABLE 7
Fungi Results from Samples of Diseased
Lemon Tissue from Ranch 10A

| | % of fungal | Plant | Reads |
|---|---|---|---|
| Name of fungi | reads | Pathogen | count |
| Aureobasidium spp. | 31.6 | Opportunistic | Very high |
| Atradidymella spp. | 19.7 | No | |
| Sclerostagonospora spp. | 17.8 | No | |
| Cladosporium spp. | 9.4 | Opportunistic | High |
| Alternaria spp. | 6.8 | Yes | High |
| Erythrobasidium spp. | 6.3 | No | |
| Neosetophoma spp. | 3.5 | No | |
| Cryptococcus spp. | 2.1 | No | |
| Derxomyces spp. | 1.2 | No | |
| Phaeosphaeria spp. | 1.1 | No | |
| Dioszegia spp. | 0.6 | No | |

TABLE 8
Bacterial Results from Samples of
Diseased Lemon Tissue from Ranch 5

| | % of bacterial | Plant | Reads |
|---|---|---|---|
| Name of bacteria | reads | Pathogen | count |
| Methylobacterium goesingense | 22.2 | No | |
| Sphingomonas Sp. | 28.2 | No | |
| Herbaspirillum rhizosphaerae | 10.8 | No | |
| Hymenobacter metalli | 10.2 | No | |
| Sodalinema Sp. | 9.4 | No | |
| Massilia Sp. | 7.0 | No | |
| Curtobacterium spp. | 4.9 | Yes | Low |
| Hymenobacter ginkgonis | 3.0 | No | |
| Okeania Sp. | 2.2 | No | |
| Novosphingobium huizhouense | 2.0 | No | |

TABLE 9
Fungi Results from Samples of Diseased
Lemon Tissue from Ranch 5

| | % of fungal | Plant | Reads |
|---|---|---|---|
| Name of fungi | reads | Pathogen | count |
| Cladosporium spp. | 53.4 | Opportunistic | Very high |
| Aureobasidium spp. | 17.0 | Opportunistic | High |
| Alternaria spp. | 11.4 | Yes | High |
| Dioszegia Sp. | 7.8 | No | |
| Neosetophoma samarorum | 2.9 | No | |
| Cryptococcus carnescens | 2.7 | No | |
| Buckleyzyma Sp. | 2.5 | No | |
| Symmetrospora coprosmae | 1.0 | No | |
| Rigidoporus microporus | 0.6 | No | |
| Erythrobasidium Sp. | 0.4 | No | |

TABLE 10
Bacterial Results from Samples of
Healthy Lemon Tissue from Ranch 10

| Name of bacteria | % of bacterial reads | Plant Pathogen |
|---|---|---|
| Sphingomonas cynarae | 31.0 | No |
| Massilia aurea | 69.0 | No |

TABLE 11
Fungi Results from Samples of Healthy
Lemon Tissue from Ranch 10

| | % of fungal | Plant | Reads |
|---|---|---|---|
| Name of fungi | reads | Pathogen | count |
| Aureobasidium spp. | 84.8 | Opportunistic | Low |
| Cladosporium cladosporioides | 6.0 | Opportunistic | Very low |
| Cryptococcus carnescens | 5.6 | No | |
| Alternaria spp. | 1.9 | Yes | Traces |
| Buckleyzyma Sp. | 1.7 | No | |

TABLE 12
Bacterial Results from Insect Traps of Ranch 5

| | % of bacterial | Plant | Reads |
|---|---|---|---|
| Name of bacteria | reads | Pathogen | count |
| Morganella spp. | 35.3% | No | |
| Commensalibacter intestini | 11.0% | No | |
| Serratia spp. | 7.9% | No | |
| Yersinia spp. | 12.2% | No | |
| Klebsiella pneumoniae | 4.5% | No | |
| Alteracholeplasma palmae | 4.0% | No | |
| Proteus mirabilis | 3.3% | No | |
| Pectobacterium spp. | 1.1% | Yes | Low |
| Others | 20.8% | No | |

TABLE 13
Fungi Results from Insect Traps of Ranch 5

| | % of fungal | Plant | |
|---|---|---|---|
| Name of fungi | reads | Pathogen | |
| Cladosporium spp. | 49.9% | Opportunistic | High |
| Aureobasidium spp. | 27.1% | Opportunistic | High |
| Cladobotryum or | 16.9% | No | |
| Hypomyces spp. | | | |
| Saccharomyces spp. | 7.7% | No | |
| Alternaria spp. | 5.8% | Yes | Low |
| Candida spp. | 5.3% | No | |
| Kluyveromyces spp. | 3.0% | No | |
| Fusarium spp. | 0.5% | Yes | Traces |
| Others | 0.6% | No | |

TABLE 14
Bacterial Results from Insect Traps of Ranch 10

| | % of bacterial | Plant | Reads |
|---|---|---|---|
| Name of bacteria | reads | Pathogen | count |
| Pseudomonas spp. | 15.6% | Yes | High |
| Serratia spp. | 15.2% | No | |
| Sphingobacterium spp. | 9.9% | No | |
| Euryhalinema spp. | 5.1% | No | |
| Wohlfahrtiimonas spp. | 4.3% | No | |
| Flavobacterium baculatum | 3.7% | No | |
| Proteus spp. | 3.1% | No | |
| Myroides guanonis | 2.3% | No | |
| Stenotrophomonas rhizophila | 1.7% | No | |
| Vagococcus penaei | 1.5% | No | |
| Massilia aurea | 1.4% | No | |
| Others | 36.4% | No | |

TABLE 15
Fungi Results from Insect Traps of Ranch 10

| | % of fungal | Plant | |
|---|---|---|---|
| Name of fungi | reads | Pathogen | |
| Cladosporium spp. | 62.5% | Opportunistic | Very high |
| Alternaria spp. | 21.4% | Yes | High |
| Aureobasidium spp. | 5.8% | Opportunistic | Low |
| Atradidymella spp. | 3.0% | No | |
| Mucor circinelloides f. | 1.6% | No | |
| circinelloides | | | |
| Fusarium spp. | 1.3% | Yes | Traces |
| Stemphylium vesicarium | 0.9% | No | |
| Cryptococcus carnescens | 1.2% | No | |
| Epicoccum nigrum | 0.8% | No | |
| Others | 1.5% | No | |

The microbial diversity between the ‘diseased’ and the ‘healthy’ lemon trees were compared against each other. Similarly, the microbial diversity of the ‘diseased’ lemon trees were compared against that of the insect traps from the ranches in which the ‘diseased’ trees were found. If there were common pathogenic bacteria or fungi present between the groups compared, it is likely that they might be implicated in the rot disease observed.

### Example 3: Case Study of Diagnosis of a complex disease in a Lemon orchard

Background: a Tango mandarin orchard has been experiencing rapid decay in 20% of the trees. The rootstock used for the trees is C35, upon which the particular variety of Tango mandarin was grafted on. This orchard has gone through several transitions in farming methods. The orchard has also experienced severe rain and flooding during the 2022 season. Several fungicides have been used in the past, but the problem continues to persist.

### Methodology:

Samples from 4 to 5 different parts of the Tango mandarin trees were obtained from 4 diseased & 2 healthy trees and analyzed for the presence of all fungi and bacteria through the processes implemented by a system of the disclosure. The analysis triangulated the root cause based on differential taxa distribution between them. The analysis optionally considered the geographical presence and symptoms observed. The following table describes the plant parts from which samples are collected for the tests.

TABLE 16

| | |
|---|---|
| Sample | Roots collected at a depth of 6 inches and at a distance of 12 |
| | inches from the trunk in four directions surrounding the tree. |
| Roots | Leaves and shoots of suckers growing from the rootstock. |
| Rootstock | Bark and xylem tissue collected from the graft union between |
| | the rootstock and the scion. |
| Graft | Bark and xylem tissue collected from the main trunk of the |
| | Tango variety above the graft union. |
| Bark | Leaves and shoots from the canopy of the scion (Tango). |
| Shoots | For each sample, four subsamples were collected at a depth of |
| | 6 inches and at a distance of 12 inches from the trunk in four |
| | directions surrounding the tree. |
| Soil | Roots collected at a depth of 6 inches and at a distance of 12 |
| | inches from the trunk in four directions surrounding the tree. |

For the pan-fungal, pan-bacterial taxonomical identification, the universally accepted genomic markers such as ITS region and 16SrRNA gene were amplified from the total extracted DNA obtained from the sample and the amplicons were sequenced with the highly accurate short-read sequencing by synthesis. The plant parts that were sent in separate sample bags were thawed first and properly decontaminated using 70% ethanol before sampling for the test. Individual samples were washed with molecular biology grade water to remove impurities/dirt that might have come through during sample collection. Approximately 5g of plant tissue sub-sampled from different parts of the same sample was placed in a sample lysis tube containing lysis buffer and ceramic beads. Similarly, 10g of soil sub-sampled after mixing the entire 100g of soil sent was 6 placed in the sample lysis tube. This was repeated for all samples. All sample lysis tubes were then placed in a ultrafast homogenizer FastPrep (from MP Biomedicals) for 1 min. The lysate collected in each of the lysis tubes was carefully aspirated out and DNA extraction was performed using the respective QuickDNA miniprep kits from Zymo Research. The extracted DNA was checked for quality & quantity. 11

10 ul of the purified DNA was then used to selectively amplify the 16SrRNA and ITS regions in separate target capture PCR reactions for each sample. More than 85% of these regions are covered in the Pest-Seq assay through multiple primer sets. The amplicons from each of the samples were cleaned up using a bead-based purification method, attached to unique sample-level molecular indices, followed by Illumina sequencing adapters and subsequently sequenced on MiSeq sequencer using the 2×250 bp v2 nano kit for approximately 27 hours. Multiple run-level controls were included in this sequencing run as well. The FASTQ data from the sequencer was then analyzed using the “Screening system” described in the claim 31 to provide sample-level identification results. The results were then manually verified to create a sample-level taxonomical ID report. The sample level taxonomic reports for all 32 samples were then collated and filtered for operational taxonomic units (OTUs) with >=95% sequence identity and >=1% abundance in the sample. The differential distribution between different sample types and diseased vs healthy samples was analyzed to identify the root cause for the Tango mandarin tree disease.

### Results

The tables with the percentage distribution of different genera in the “Roots” for the 6 Tango mandarin trees are shown below.

TABLE 17

| | D1- | D2- | D3- | D4- | H1- | H2- |
|---|---|---|---|---|---|---|
| Genus only | roots | roots | roots | roots | roots | roots |
| Certobasidum | 10.46 | | 12.05 | 1.42 | 3.03 | |
| Fusarium | 1.27 | | | 14.48 | 12.15 | 15.79 |
| Ilyonectria | | | 1.06 | | | |
| Neobacillus | | 3.3 | 3.67 | | | |
| Nigrospora | | | | 4.39 | | |
| Okeania | | | | | 1.32 | |
| Oryza | | | 1.33 | | | |
| Peribacillus | 2.73 | 3.37 | | | | 2.61 |
| Priestia | | 3.2 | | | | 1.85 |
| Pseudomonas | | | | | 1.21 | |
| Steroidobacter | | | | 2.31 | 1.35 | |
| Tricchocladium | | | 5.17 | 4.27 | | |

The tables with the percentage distribution of different genera in the “Graft unions” for the 6 Tango mandarin trees are shown below.

TABLE 18

| | D1- | D2- | D3- | D4- | H1- | H2- |
|---|---|---|---|---|---|---|
| Genus only | Graft | Graft | Graft | Graft | Graft | Graft |
| Acremonium | | | 1.53 | | | |
| Alternaria | 3.51 | 1.17 | 18.75 | | | 1.77 |
| Aureobasidium | 3.83 | | 1.89 | | 4.15 | 5.3 |
| Cldosporium | 1.42 | | | | | |
| Cryptococcus | 3.08 | | | | | |
| Dlhwksworthia | | | | | | |
| Euryhalinema | | 18.32 | | | | |
| Fusarium | | 11.15 | 7.14 | 12.97 | | |
| Massillia | | 1.42 | | | | |
| Microlunatus | | 1.12 | | 1.02 | | |
| Neosetophoma | 10.25 | 4.13 | 7.41 | 6.43 | 15.18 | 14.22 |
| Pedobacter | | 1.48 | | | | |
| Phoma | 9.8 | 3.99 | 5.05 | | | |
| Pseudomonas | | | | 1.74 | | |
| Pseudostichoccoccus | | | | 1.92 | | |
| Rhizobium | | | | 1.78 | | |
| Sarocladium | | | | 2.76 | | |
| Trebouxia | 3.98 | 3.55 | | 4.43 | 2.29 | |
| Veronaea | | | | 4.47 | | |

The tables with the percentage distribution of different genera in the “barks” for the 6 Tango mandarin trees are shown below.

TABLE 19

| | D1- | D2- | D3- | D4- | H1- | H2- |
|---|---|---|---|---|---|---|
| Genus only | Bark | Bark | Bark | Bark | Bark | Bark |
| Alternaria | | 3.19 | | 7.87 | | 10.12 |
| Aureobasidium | | | | | 7.38 | 18.82 |
| Blastococcus | | | | 1.89 | | |
| Cladosporium | | 2.03 | | 2.73 | | 10.21 |
| Deinococcus | | | | 2.37 | | |
| Erwinia | | | | | 2.63 | |
| Klenkia | | | | | | 2.39 |
| Methyllobacterium | | | | | | 2.3 |
| Microlunatus | | | 3.76 | 3.64 | 2.18 | |
| Neosetophoma | | 28.54 | 39.09 | 17.69 | 25.51 | |
| Pantoea | | | | | 2.02 | |
| Phoma | 25.16 | | | 12.05 | 13.53 | |
| Pseudostichococcus | | 8.23 | | 19.89 | | |
| Roseomonas | 1.85 | | | | | |
| Sphingomonas | | | 2.58 | | | 2.29 |
| Trebouxia | | 6.76 | | 14.47 | 8.38 | 3.03 |
| Wojnowiciella | | 3 | | | | |

The tables with the percentage distribution of different genera in the “shoots” for the 6 Tango mandarin trees are shown below.

TABLE 20

| | D1- | D2- | D3- | D4- | H1- | H2- |
|---|---|---|---|---|---|---|
| Genus only | Shoots | Shoots | Shoots | Shoots | Shoots | Shoots |
| Alternaria | 13.99 | 2.84 | 4.83 | 7.02 | 8.13 | |
| Aureobasidium | 7.04 | 9.07 | 4.88 | 3.65 | 3.75 | 11.36 |
| Cladosporium | 1.26 | 1.88 | | 3.81 | | 2.12 |
| Colletotrichum | 1.96 | 8.27 | 14.67 | 16.3 | 9.88 | |
| Cryptococcus | | | | | | 1.12 |
| Dioszegia | | | | | | |
| Massillia | 1.72 | | | | | |
| Neosetophoma | | | | 2.83 | | |

The tables with the percentage distribution of different genera in the “soil” for the 6 Tango mandarin trees are shown below.

TABLE 21

| | D1- | D2- | D3- | D4- | H1- | D1- |
|---|---|---|---|---|---|---|
| Genus | Soil | Soil | Soil | Soil | Soil | Soil |
| Acidibacter | 0.99 | | | | | |
| Aciditerrimonas | | 1.03 | | | | |
| Agrobacterium | | | | | | 1.55 |
| Arenimicrobium | | | | | | |
| Conexibacter | 1.07 | | | | | |
| Cupriavidus | | | | | 1.28 | |
| Cytobacillus | | | | 1.43 | | |
| Dongia | 1.65 | | 1.29 | | | |
| Fusarium | 15.89 | 1.34 | 7.79 | 1.23 | 4.12 | 4.85 |
| Gaiella | | | 3.95 | 3.02 | | |
| Gemmatirosa | | | | | 3.94 | |
| Halopseudomonas | | | | | | 1.02 |
| Humicola | 1.17 | | | | | |
| Limisphaera | | | | | 1.66 | |
| Longimicrobium | 1.13 | | | | | |
| Luteitalea | | | | | | 2.73 |
| Neobacillus | | | | 1.02 | | 1.06 |
| Nitrospira | 1.48 | 3.59 | 1.51 | 1.71 | 1.39 | 1.18 |
| Pedomicrobium | | | | | | 1.15 |
| Peribacillus | | | | 3.6 | | |
| Povalibacter | 1.03 | 1.83 | 1.61 | | | |
| Pseudogulbenkiania | | 1.16 | | | | |
| Pseudomonas | | | | 3.01 | 11.77 | 7.13 |
| Rhabdothermincola | 2.89 | 1.8 | | | 2.61 | 2 |
| Sphingomonas | 1.41 | 1.41 | 1.33 | 1 | | 0.97 |
| Steroidobacter | | | 3.01 | | | |
| Tepidiforma | | | | 1.37 | | |
| Tepidisphaera | | | 1.2 | | | |
| Thermoleophilum | | | 1.17 | | | |
| Thiobacter | | 1.34 | | 1.05 | | |
| Vitis | | | | | | 1.07 |
| Vitreimonas | | 1.2 | | | | |

Notably, the two “Rootstock” samples (D1 & D2) tested were devoid of any bacteria and fungi and were “clean”.

Penicillium spp., Xanthomonas spp., Candidatus Liberibacter (HLB), and Xylella spp. Surprisingly, the following pathogens were found in different parts of the Tango mandarin trees: Fusarium spp., Phoma spp., Colletotrichum boninense, Alternaria spp., Aureobasidium pullalans. Among these, Fusarium spp. and Phoma spp. are present only in the graft union of the diseased trees and not on the healthy ones. Other pathogens do not show such clear differentiation in prevalence between the diseased and healthy trees. This is a notable results because Phoma spp. related Mal Secco disease is not yet reported in the U.S. Based on geographic considerations along, thi pathogen would not have been identified as a cause of the decay. Nonetheless, in view of the clear detection of the pathogen, specific symptoms observed, and the microorganism known ability to cause gradual full tree dieback, we conclude that Fusarium spp. is likely the root cause for the Tango mandarin decline in Roadrunner 3 orchard. This Fusarium-related dry root rot disease is increasingly becoming common in Citrus trees in the United States, despite not being readily associated with this geographic area.

Though Fusarium spp. is present in most of the soil samples, including the ones that were collected from underneath the ‘healthy’ trees, it is also not found in the rootstock. This suggests that Fusarium has perhaps propagated from the soil to the graft unions of the diseased trees, not through the root stock, but through injury-related exposure of the graft union during a rainy or windy season. Fusarium is also not present in the barks or shoots.

We further observe that other opportunistic pathogens such as Alternaria spp., Colletotrichum boninense, Phoma spp., and Aureobasidium pullalans present in the diseased trees may have played a role in the weakening and gradual decay of the tree along with Fusarium spp.

While Fusarium spp. is also found in the soil underneath the “Healthy” trees, they have not found their way to the graft union. This may be fortuitous coincidence. Other interesting and useful observation is that the soil underneath the healthy trees have higher % of Pseudomonas spp., which is either totally absent or present in very low amounts in the soil of the diseased trees. Pseudomonas spp. and other Bacillus spp. in soil are well known to provide antifungal resistance to the plants through the production of bioactives and antimicrobial compounds^{3,4}. Perhaps, this could also be the reason why many of the trees are not impacted by Fusarium spp.

This may also suggest that supplementing the soil with these bacteria or similar bio-fungicides could provide resistance to soil fungi and help to reclaim the orchard from such future threats.

This example demonstrates a case use of the process for screening an agricultural unit for one or more taxa of the disclosure. In this case, the system may output a qualitative risk score, namely the presence of Fusarium spp. In the diseased trees.

### Example 4: Using soil microbiome to decide on the best soil treatment for a Walnut farm

Background: walnut grower wished to evaluate 3 different soil treatments and decide which one(s) to use among them for improving the soil health of this farm. The 3 treatments were coded as follows:
White-Regular Compost
Green-Whole tree chipped mulch, combined with Compost
Red-Green treatment+Mulched root mass raked up from surface to 4-ft depth

### Experiments

For the pan-fungal, pan-bacterial taxonomical identification, the universally accepted genomic markers such as ITS region and 16SrRNA gene were amplified from the total extracted DNA obtained from the sample and the amplicons were sequenced with the highly accurate short-read sequencing by synthesis. The soil from the 3 treatments sent in separate sample bags were thawed first and properly decontaminated using 70% ethanol before sampling for the test. The soil in individual sample bags were mixed well with a large spatula and any large clumps were broken down. Approximately 1g of soil sub-sampled from different parts of the sample bag was placed in a sample lysis tube containing lysis buffer and ceramic beads. This was repeated for all samples. All sample lysis tubes were then placed in an ultrafast homogenizer FastPrep (from MP Biomedicals) for 1 min. The lysate collected in each of the lysis tubes was carefully aspirated out and DNA extraction was performed using Zymobiomics kit from Zymo Research. The extracted DNA was checked for quality & quantity. 10 ul of the purified DNA was then used to selectively amplify the 16SrRNA and ITS regions in separate target capture PCR reactions for each sample. More than 85% of these regions are covered in the Pest-Seq assay through multiple primer sets. The amplicons from each of the samples were cleaned up using a bead-based purification method, attached to unique sample-level molecular indices, followed by Illumina sequencing adapters and subsequently sequenced on MiSeq sequencer using the 2×250 bp v2 nano kit for approximately 27 hours. Multiple run-level controls were included in this sequencing run as well.

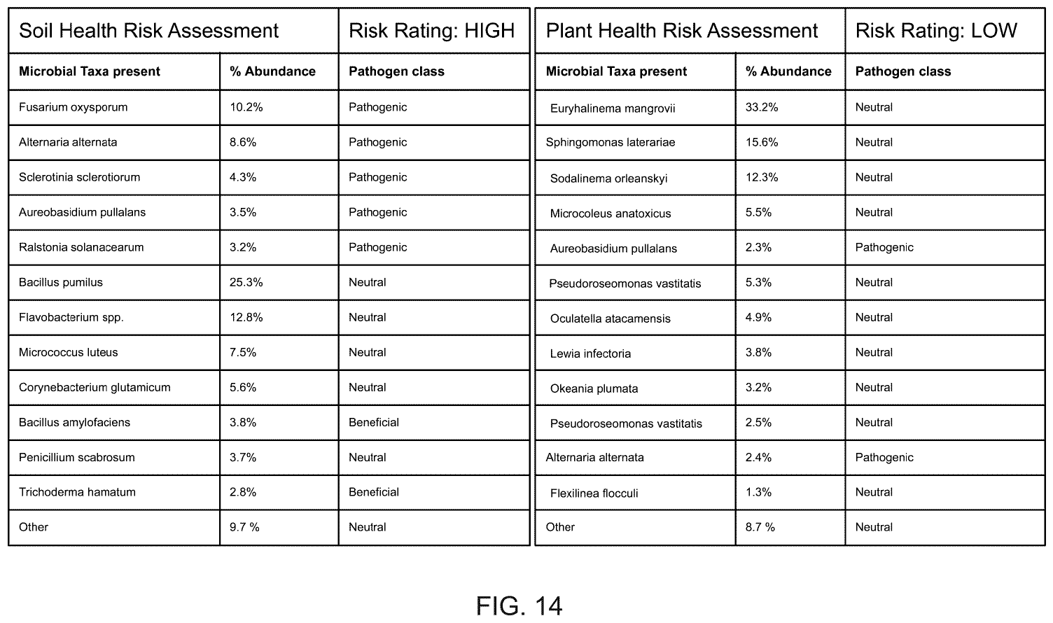

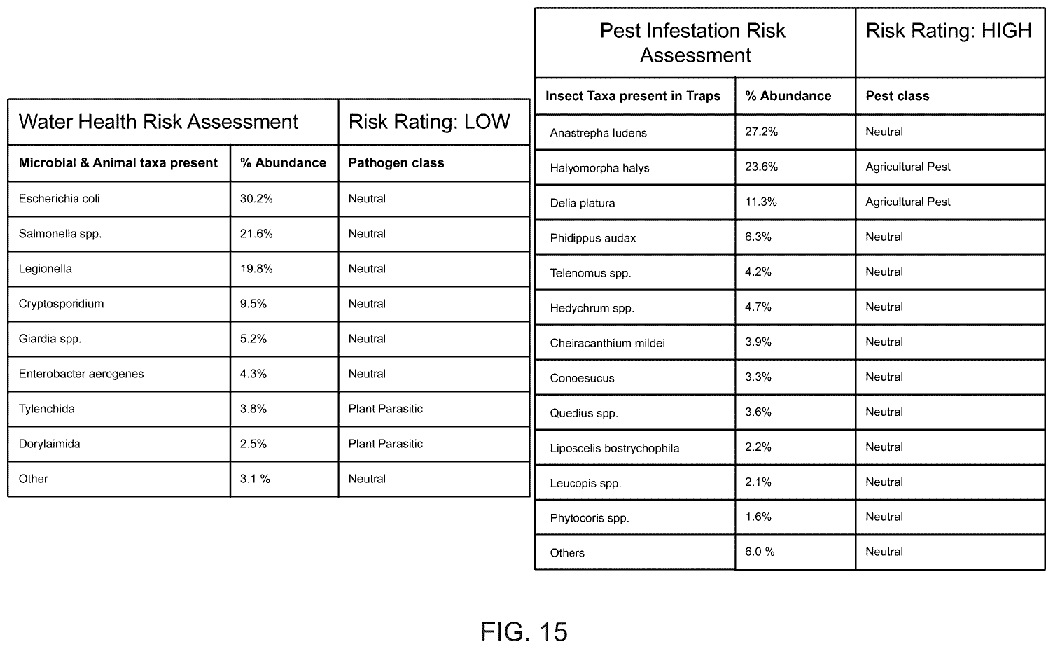

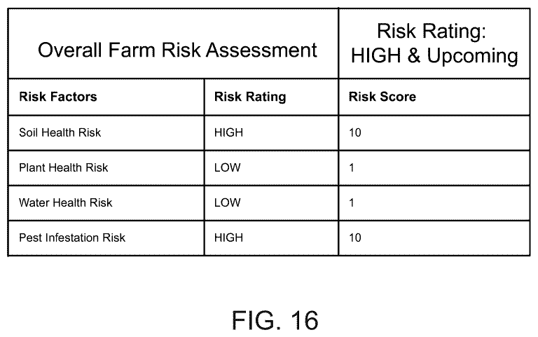

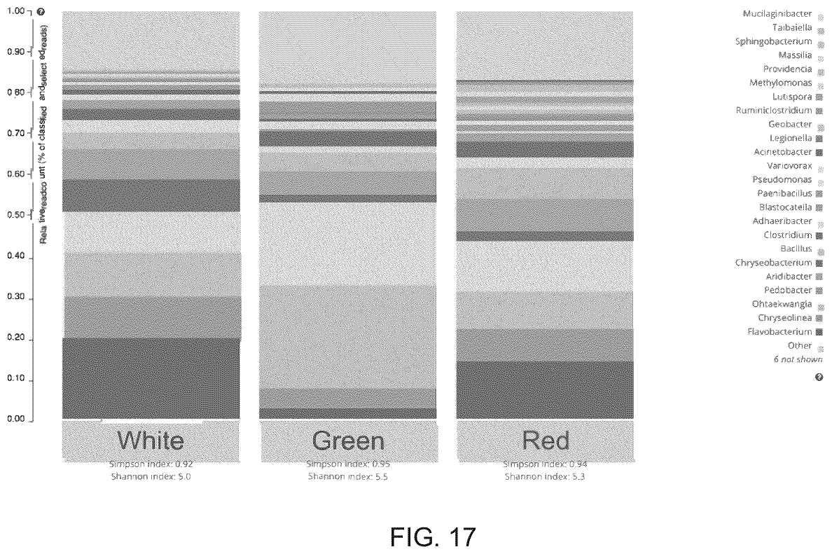

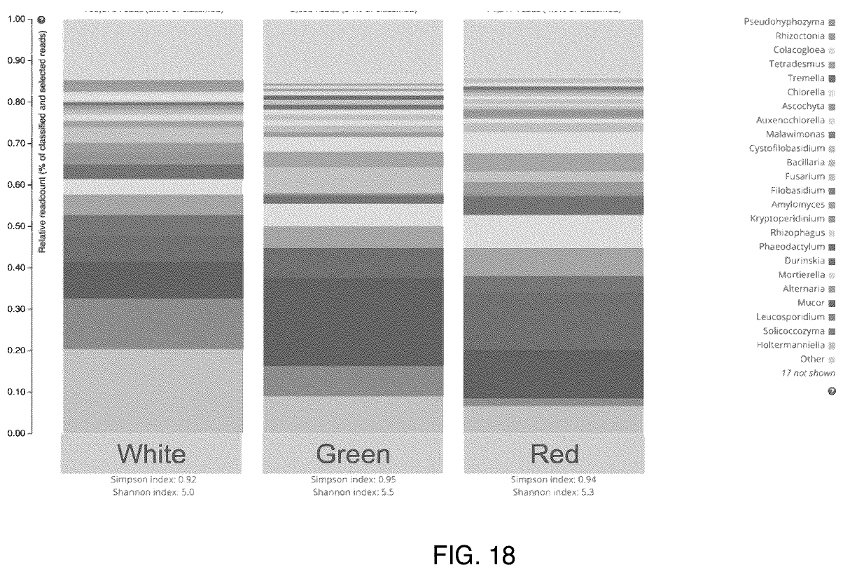

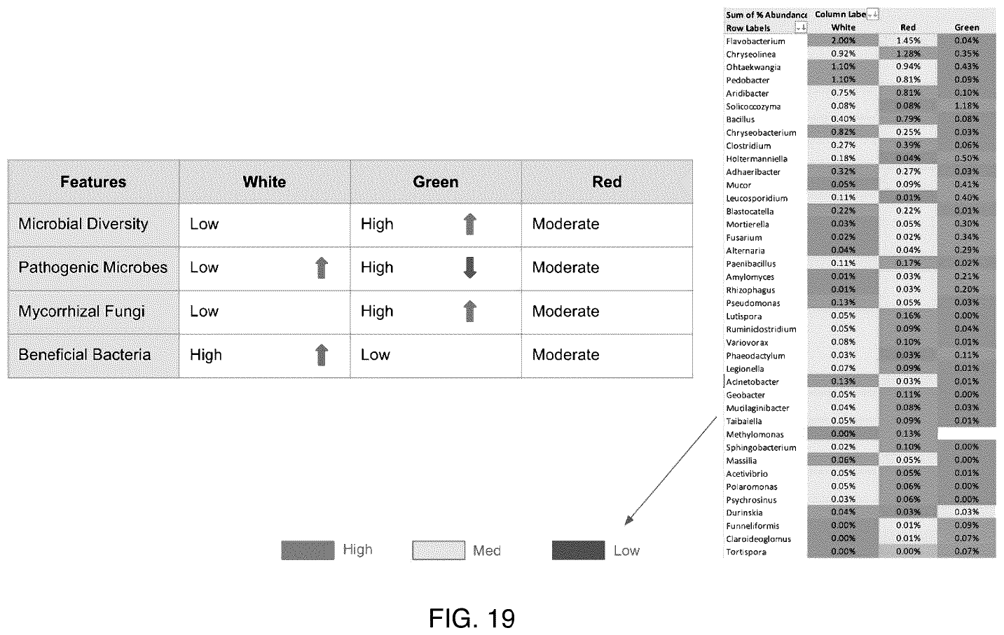

The FASTQ data from the sequencer was then analyzed using the “Screening system” described in the claim 31 to provide sample-level identification results. The results were then manually verified to create a sample-level taxonomical ID report. The sample level taxonomic reports for all 3 samples were then collated and filtered for operational taxonomic units (OTUs) with >=95% sequence identity and >=1% abundance in the sample. The relative abundance of bacterial general between the soil samples is depicted as a heat map on (FIG. 14). The relative abundance of bacterial general between the soil samples is depicted as a heat map on (FIG. 15). The relative abundance of microbes between the soil types is depicted as a heat map on (FIG. 16)

For walnut farming, the ideal soil microbiome should promote:
1. Strong root health and mycorrhizal symbiosis, especially arbuscular mycorrhizal (like Rhizophagus), which enhance phosphorus and micronutrient uptake;
2. Suppression of root and vascular pathogens; walnuts are vulnerable to Phytophthore, fusarium, and armillaria;
3. sustained soil fertility and organic matter turnover; via beneficial decomposers and nitrogen cyclers.
The following treatments for walnut farming were weighed, and a risk assessment score ws determined.

TABLE 22

| | Importance for | Best | |
|---|---|---|---|
| Criterion | Walnuts | treatment | Notes |
| Beneficial/harmful | High | White | Strongly suppresses |
| microbe ratio | | | harmful taxa like |
| | | | Fusarium and |
| | | | Clostridium |
| Shannon diversity | Moderate | Green | Promotes Resilience |
| index | | | and functional |
| | | | redundancy |
| Mycorrhizal fungi | critical | Green | Enhances walnut root |
| (rhizophagus) | | | nutrient uptake and |
| | | | drought tolerance |

The following soil treatment plan was proposed based on the data and the microbial profile:

Primary treatment-green. Rationale: highest mycorrhizal (Rhizophagus) presence and diversity. Recommendation: apply in early spring or post-harvest to foster microbial symbiosis.

Supplements: add arbuscular mycorrhizal inoculant (e.g., rhizophagus irregularis) at panting or drip-injection. Include biostimulants (e.g., humic acids, seawee extracts) to support funcal colonization. 3

Supportive boost-white (optional in rotation). Rationale: high suppression of pathogens like fuarium and clostridium. Use when: soil shows high pathogen pressure, waterlogging, or post-disease outbreaks. Supplement; apply biocontrol agents like Bacillus subtillis, Pseudomonas fluorescens, or Trichoderma harzianum.

While certain embodiments have been described, these embodiments have been presented by way of example only and are not intended to limit the scope of the present disclosures. Indeed, the novel methods, apparatuses, modules, instruments and systems described herein can be embodied in a variety of other forms; furthermore, various omissions, substitutions and changes in the form of the methods, apparatuses, modules, instruments and systems described herein can be made without departing from the spirit of the present disclosures. The accompanying claims and their equivalents are intended to cover such forms or modifications as would fall within the scope and spirit of the present disclosures.

1. A process for screening an agricultural unit for a health risk to the agricultural unit, comprising:
generating a plurality of nucleic acid sequencing reads from one or more sample(s) from the agricultural unit;
screening the plurality of nucleic acid sequencing reads derived from the sample(s) gathered from the agricultural unit for a pattern that identifies the health risk to the agricultural unit, whereby the screening is performed by a computer program model trained to identify the pattern from a plurality of features, whereby one or more features comprises the abundance of the plurality of taxa found in the agricultural unit,
outputting the risk score that is predictive of the health risk to the agricultural unit based on the screening.

2. The process of claim 1, whereby the health risk to the agricultural unit is a risk of crop failure or damage.

3. The process of claim 1, wherein the health risk to the agricultural unit is a risk of a pest infestation.

4. The process of claim 1, wherein the health risk to the agricultural unit is a risk of a depletion of organic matter.

5. The process of claim 1, wherein the health risk to the agricultural unit is a risk of a soil mineralization.

6. The process of claim 1, wherein the health risk to the agricultural unit is a risk of depletion of beneficial microbiota from the soil or from within the crop.

7. The process of claim 1, wherein the plurality of nucleic acid sequences extracted from the agricultural unit comprises:
a plurality of nucleic acid sequences extracted from at least one arthropod;
a plurality of nucleic acid sequences extracted from at least one nematode;
a plurality of nucleic acid sequences extracted from at least one bacterium;
a plurality of nucleic acid sequences extracted from at least one fungus/mold/yeast;
a plurality of nucleic acid sequences extracted from at least one protozoa;
a plurality of nucleic acid sequences extracted from at least one archaea; and/or
a plurality of nucleic acid sequences extracted from at least one virus.

8. The process of claim 7, wherein the computer program model identifies a taxa in the agricultural unit based on a plurality of chromosomal or mitochondrial metabarcode nucleic acid sequences selected from the group comprising Cytochrome Oxidase I, Cytochrome b, 16S rRNA, ITS in the plurality of nucleic acid sequences extracted from the at least one arthropod.

9. The process of claim 7, wherein the computer program model identifies a taxa in the agricultural unit based on a plurality of chromosomal or mitochondrial metabarcode nucleic acid sequences selected from the group comprising Cytochrome Oxidase I, Cytochrome b, 18S small subunit rRNA, and 28S large subunit rRNA in the plurality of nucleic acid sequences extracted from the at least one nematode.

10. The process of claim 7, wherein the computer program model identifies a taxa in the agricultural unit based on a plurality of chromosomal or mitochondrial metabarcode nucleic acid sequences selected from the group comprising 16S rRNA, ITS in the plurality of nucleic acid sequences extracted from the at least one bacterium or archaea.

11. The process of claim 7, wherein the computer program model identifies a taxa in the agricultural unit based on a plurality of chromosomal or mitochondrial metabarcode nucleic acid sequences selected from the group comprising 16S rRNA and ITS in the plurality of nucleic acid sequences extracted from the at least one fungus.

12. The process of claim 7, wherein the computer program model identifies a taxa in the agricultural unit based on a plurality of chromosomal or mitochondrial metabarcode nucleic acid sequences selected from the group comprising 16S rRNA, 18S rRNA in the plurality of nucleic acid sequences extracted from the at least one protozoa.

13. The process of claim 7, wherein the computer program model identifies a virus in the agricultural unit based on targeted amplicon sequencing assay.

14. The process of claim 9, whereby the process identifies larvae, nymph, eggs, and mature taxa.

15. The process of claim 7, wherein the processing of the sample of the agricultural unit comprises isolating a plurality of nucleic acids from the sample and processing the plurality of nucleic acids for sequencing.

16. The process of claim 15, wherein the extracted nucleic acids are enriched via primer-based target amplification.

17. The process of claim 16, wherein the set of primers for primer-based target amplification comprises markers for insecticide resistance, antibiotic, and/or anti-fungal resistance marker.

18. The process of claim 15, wherein the extracted nucleic acids are enriched via an amplification method selected from the group comprising of: PCR, RPA, NASBA, LAMP, TMA, MDA, RCA, and HAD amplification methods.

19. The process of claim 15, wherein the extracted nucleic acids are enriched via hybridization-based target amplification using a panel of capture probes selected from the group comprising of: DNA capture probes, ssDNA capture probes, and RNA capture probes.

20. The process of claim 15, wherein the hybridization-based target amplification is a solid-phase target capture assay, a bead-capture assay, or a liquid-phase capture assay.
